SEQUENCE LISTING (136 sequences)

SEQ ID NO: 1 - protein, 311 aa, Artificial Sequence
  Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 2 - protein, 274 aa, Artificial Sequence
  Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 117 aa, Artificial Sequence
  Synthetic
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGN

SEQ ID NO: 4 - protein, 116 aa, Artificial Sequence
  Synthetic
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSG

SEQ ID NO: 5 - protein, 315 aa, Artificial Sequence
  Synthetic
MSIGLLCCAALSLLWAGPVNAGVTQTPKFQVLKTGQSMTLQCAQDMNHEYMSWYRQDPGMGLRLIHYSVXXXXTDQGEVPNGYNVSRSTTEDFPLRLLSAAPSQTSVYFCASSYXXXTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLTVL

SEQ ID NO: 6 - protein, 274 aa, Artificial Sequence
  Synthetic
METLLGLLILWLQLQWVSSKQEVTQIPAALSVPEGENLVLNCSFTDSAIYNLQWFRQDPGKGLTSLLLIXXXQREQTSGRLNASLDKSSGRSTLYIAASQPGDSATYLCAVRXXXXGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 7 - protein, 117 aa, Artificial Sequence
  Synthetic
MSIGLLCCAALSLLWAGPVNAGVTQTPKFQVLKTGQSMTLQCAQDMNHEYMSWYRQDPGMGLRLIHYSVXXXXTDQGEVPNGYNVSRSTTEDFPLRLLSAAPSQTSVYFCASSYXXX

SEQ ID NO: 8 - protein, 116 aa, Artificial Sequence
  Synthetic
METLLGLLILWLQLQWVSSKQEVTQIPAALSVPEGENLVLNCSFTDSAIYNLQWFRQDPGKGLTSLLLIXXXQREQTSGRLNASLDKSSGRSTLYIAASQPGDSATYLCAVRXXXX

SEQ ID NO: 9 - protein, 306 aa, Artificial Sequence
  Synthetic
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 10 - protein, 112 aa, Artificial Sequence
  Synthetic
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCAS

SEQ ID NO: 11 - protein, 306 aa, Artificial Sequence
  Synthetic
MRIRLLCCVAFSLLWAGPVIAGITQAPTSQILAAGRRMTLRCTQDMRHNAMYWYRQDLGLGLRLIHYSNXXXXTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 12 - protein, 113 aa, Artificial Sequence
  Synthetic
MRIRLLCCVAFSLLWAGPVIAGITQAPTSQILAAGRRMTLRCTQDMRHNAMYWYRQDLGLGLRLIHYSNXXXXTGKGEVPDGYSVSRANTDDFPLTLASAVPSQTSVYFCASS

SEQ ID NO: 13 - protein, 273 aa, Artificial Sequence
  Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 114 aa, Artificial Sequence
  Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVN

SEQ ID NO: 15 - protein, 268 aa, Artificial Sequence
  Synthetic
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 311 aa, Artificial Sequence
  Synthetic
MSIGLLCCAALSLLWAGPVNAGVTQTPKFQVLKTGQSMTLQCAQDMNHEYMSWYRQDPGMGLRLIHYSVAIGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 17 - protein, 4 aa, Artificial Sequence
  Synthetic
GAGI

SEQ ID NO: 18 - protein, 4 aa, Artificial Sequence
  Synthetic
GAGT

SEQ ID NO: 19 - protein, 4 aa, Artificial Sequence
  Synthetic
GIQI

SEQ ID NO: 20 - protein, 4 aa, Artificial Sequence
  Synthetic
GIGT

SEQ ID NO: 21 - protein, 4 aa, Artificial Sequence
  Synthetic
GAQT

SEQ ID NO: 22 - protein, 4 aa, Artificial Sequence
  Synthetic
AIQI

SEQ ID NO: 23 - protein, 4 aa, Artificial Sequence
  Synthetic
AIGT

SEQ ID NO: 24 - protein, 4 aa, Artificial Sequence
  Synthetic
GIQT

SEQ ID NO: 25 - protein, 4 aa, Artificial Sequence
  Synthetic
AIQT

SEQ ID NO: 26 - protein, 4 aa, Artificial Sequence
  Synthetic
PTSG

SEQ ID NO: 27 - protein, 4 aa, Artificial Sequence
  Synthetic
PLYG

SEQ ID NO: 28 - protein, 4 aa, Artificial Sequence
  Synthetic
PASG

SEQ ID NO: 29 - protein, 4 aa, Artificial Sequence
  Synthetic
PLSG

SEQ ID NO: 30 - protein, 4 aa, Artificial Sequence
  Synthetic
PESG

SEQ ID NO: 31 - protein, 4 aa, Artificial Sequence
  Synthetic
PTAG

SEQ ID NO: 32 - protein, 4 aa, Artificial Sequence
  Synthetic
PTLG

SEQ ID NO: 33 - protein, 4 aa, Artificial Sequence
  Synthetic
PTYG

SEQ ID NO: 34 - protein, 4 aa, Artificial Sequence
  Synthetic
PTEG

SEQ ID NO: 35 - protein, 4 aa, Artificial Sequence
  Synthetic
PTKG

SEQ ID NO: 36 - protein, 4 aa, Artificial Sequence
  Synthetic
PLLD

SEQ ID NO: 37 - protein, 4 aa, Artificial Sequence
  Synthetic
PLLG

SEQ ID NO: 38 - protein, 4 aa, Artificial Sequence
  Synthetic
PEYG

SEQ ID NO: 39 - protein, 4 aa, Artificial Sequence
  Synthetic
LPSG

SEQ ID NO: 40 - protein, 4 aa, Artificial Sequence
  Synthetic
TAGT

SEQ ID NO: 41 - protein, 4 aa, Artificial Sequence
  Synthetic
VAGT

SEQ ID NO: 42 - protein, 4 aa, Artificial Sequence
  Synthetic
TMGT

SEQ ID NO: 43 - DNA, 46 bp, Artificial Sequence
  Synthetic
atattaatacgactcactatagggcaccatggagaccctgctgggc

SEQ ID NO: 44 - DNA, 20 bp, Artificial Sequence
  Synthetic
ccgcacagcgcacaggtagg

SEQ ID NO: 45 - DNA, 41 bp, Artificial Sequence
  Synthetic
cctacctgtgcgctgtgcgggccaccagcggcggcagctac

SEQ ID NO: 46 - DNA, 41 bp, Artificial Sequence
  Synthetic
cctacctgtgcgctgtgcggctgaccagcggcggcagctac

SEQ ID NO: 47 - DNA, 41 bp, Artificial Sequence
  Synthetic
cctacctgtgcgctgtgcggcctgccagcggcggcagctac

SEQ ID NO: 48 - DNA, 41 bp, Artificial Sequence
  Synthetic
cctacctgtgcgctgtgcggcctctgagcggcggcagctac

SEQ ID NO: 49 - DNA, 41 bp, Artificial Sequence
  Synthetic
cctacctgtgcgctgtgcggccttacagcggcggcagctac

SEQ ID NO: 50 - DNA, 41 bp, Artificial Sequence
  Synthetic
cctacctgtgcgctgtgcggcctgagagcggcggcagctac

SEQ ID NO: 51 - DNA, 41 bp, Artificial Sequence
  Synthetic
cctacctgtgcgctgtgcggcctaagagcggcggcagctac

SEQ ID NO: 52 - DNA, 41 bp, Artificial Sequence
  Synthetic
cctacctgtgcgctgtgcggcctcccagcggcggcagctac

SEQ ID NO: 53 - DNA, 41 bp, Artificial Sequence
  Synthetic
cctacctgtgcgctgtgcggcctaccgccggcggcagctac

SEQ ID NO: 54 - DNA, 41 bp, Artificial Sequence
  Synthetic
cctacctgtgcgctgtgcggcctaccctgggcggcagctac

SEQ ID NO: 55 - DNA, 41 bp, Artificial Sequence
  Synthetic
cctacctgtgcgctgtgcggcctacctacggcggcagctac

SEQ ID NO: 56 - DNA, 41 bp, Artificial Sequence
  Synthetic
cctacctgtgcgctgtgcggcctaccgagggcggcagctac

SEQ ID NO: 57 - DNA, 41 bp, Artificial Sequence
  Synthetic
cctacctgtgcgctgtgcggcctaccaagggcggcagctac

SEQ ID NO: 58 - DNA, 41 bp, Artificial Sequence
  Synthetic
cctacctgtgcgctgtgcggcctacccccggcggcagctac

SEQ ID NO: 59 - DNA, 41 bp, Artificial Sequence
  Synthetic
cctacctgtgcgctgtgcggcctaccagcgccggcagctac

SEQ ID NO: 60 - DNA, 41 bp, Artificial Sequence
  Synthetic
cctacctgtgcgctgtgcggcctaccagcgagggcagctac

SEQ ID NO: 61 - DNA, 41 bp, Artificial Sequence
  Synthetic
cctacctgtgcgctgtgcggcctaccagcaagggcagctac

SEQ ID NO: 62 - DNA, 41 bp, Artificial Sequence
  Synthetic
cctacctgtgcgctgtgcggcctaccagccccggcagctac

SEQ ID NO: 63 - DNA, 45 bp, Artificial Sequence
  Synthetic
cctacctgtgcgctgtgcggcccctgctgggcggcagctacatcc

SEQ ID NO: 64 - DNA, 45 bp, Artificial Sequence
  Synthetic
cctacctgtgcgctgtgcggcccctgtacggcggcagctacatcc

SEQ ID NO: 65 - DNA, 85 bp, Artificial Sequence
  Synthetic
ttttttttttttttttttttttttttttttttttttttttttttttttttttttttttttttttttagctgctccacagccgcag

SEQ ID NO: 66 - DNA, 44 bp, Artificial Sequence
  Synthetic
atattaatacgactcactatagggatgagcatcggcctgctgtg

SEQ ID NO: 67 - DNA, 21 bp, Artificial Sequence
  Synthetic
cacagagtagtggatcagccg

SEQ ID NO: 68 - DNA, 48 bp, Artificial Sequence
  Synthetic
cggctgatccactactctgtggccgccggaatcaccgaccagggcgag

SEQ ID NO: 69 - DNA, 48 bp, Artificial Sequence
  Synthetic
cggctgatccactactctgtgggaatcggaatcaccgaccagggcgag

SEQ ID NO: 70 - DNA, 48 bp, Artificial Sequence
  Synthetic
cggctgatccactactctgtgggagcccagatcaccgaccagggcgag

SEQ ID NO: 71 - DNA, 48 bp, Artificial Sequence
  Synthetic
cggctgatccactactctgtgggagccggaaccaccgaccagggcgag

SEQ ID NO: 72 - DNA, 48 bp, Artificial Sequence
  Synthetic
cggctgatccactactctgtggccatcggaatcaccgaccagggcgag

SEQ ID NO: 73 - DNA, 48 bp, Artificial Sequence
  Synthetic
cggctgatccactactctgtggccgccggaaccaccgaccagggcgag

SEQ ID NO: 74 - DNA, 48 bp, Artificial Sequence
  Synthetic
cggctgatccactactctgtgggaatcggaaccaccgaccagggcgag

SEQ ID NO: 75 - DNA, 48 bp, Artificial Sequence
  Synthetic
cggctgatccactactctgtggccatcggaaccaccgaccagggcgag

SEQ ID NO: 76 - DNA, 84 bp, Artificial Sequence
  Synthetic
ttttttttttttttttttttttttttttttttttttttttttttttttttttttttttttttttttagccccggctgtccttcc

SEQ ID NO: 77 - DNA, 44 bp, Artificial; identical to SEQ ID NO: 66
  Synthetic

SEQ ID NO: 78 - DNA, 22 bp, Artificial Sequence
  Synthetic
atagctgctggcgcagaagtac

SEQ ID NO: 79 - DNA, 41 bp, Artificial Sequence
  Synthetic
gtacttctgcgccagcagctatgccggcaacaccggcgagc

SEQ ID NO: 80 - DNA, 41 bp, Artificial Sequence
  Synthetic
gtacttctgcgccagcagctatctgggcaacaccggcgagc

SEQ ID NO: 81 - DNA, 41 bp, Artificial Sequence
  Synthetic
gtacttctgcgccagcagctattacggcaacaccggcgagc

SEQ ID NO: 82 - DNA, 41 bp, Artificial Sequence
  Synthetic
gtacttctgcgccagcagctatgagggcaacaccggcgagc

SEQ ID NO: 83 - DNA, 41 bp, Artificial Sequence
  Synthetic
gtacttctgcgccagcagctatgtggccaacaccggcgagc

SEQ ID NO: 84 - DNA, 41 bp, Artificial Sequence
  Synthetic
gtacttctgcgccagcagctatgtgggcgccaccggcgagc

SEQ ID NO: 85 - DNA, 41 bp, Artificial Sequence
  Synthetic
gtacttctgcgccagcagctatgtgggcgacaccggcgagc

SEQ ID NO: 86 - DNA, 41 bp, Artificial Sequence
  Synthetic
gtacttctgcgccagcagctatgtgggcctgaccggcgagc

SEQ ID NO: 87 - DNA, 41 bp, Artificial Sequence
  Synthetic
gtacttctgcgccagcagctatgtgggcaagaccggcgagc

SEQ ID NO: 88 - DNA, 41 bp, Artificial Sequence
  Synthetic
gtacttctgcgccagcagctatgtgggccccaccggcgagc

SEQ ID NO: 89 - DNA, 84 bp, Artificial Sequence; identical to SEQ ID NO: 76
  Synthetic

SEQ ID NO: 90 - DNA, 50 bp, Artificial Sequence
  Synthetic
gactaattaaccctcactaaagggacaccatgggcacaaggttgttcttc

SEQ ID NO: 91 - DNA, 20 bp, Artificial Sequence
  Synthetic
gtaatggatcagcctcagcc

SEQ ID NO: 92 - DNA, 55 bp, Artificial
  Synthetic
gctgaggctgatccattactcatatgccgttaaagatactgacaaaggagaagtc

SEQ ID NO: 93 - DNA, 55 bp, Artificial
  Synthetic
gctgaggctgatccattactcatatgccgccaaagatactgacaaaggagaagtc

SEQ ID NO: 94 - DNA, 55 bp, Artificial Sequence
  Synthetic
gctgaggctgatccattactcatatgccgttgccgatactgacaaaggagaagtc

SEQ ID NO: 95 - DNA, 55 bp, Artificial Sequence
  Synthetic
gctgaggctgatccattactcatatgccgttaaagccactgacaaaggagaagtc

SEQ ID NO: 96 - DNA, 91 bp, Artificial Sequence
  Synthetic
tttttttttttttttttttttttttttttttttttttttttttttttttttttttttttttttttcagaaatcctttctcttgaccatggc

SEQ ID NO: 97 - DNA, 51 bp, Artificial Sequence
  Synthetic
gactaattaaccctcactaaagggacaccatgagaatcaggctcctgtgct

SEQ ID NO: 98 - DNA, 22 bp, Artificial Sequence
  Synthetic
tgaataatggatgagccttagc

SEQ ID NO: 99 - DNA, 56 bp, Artificial Sequence
  Synthetic
gctaaggctcatccattattcaaatgccgcaggtaccactggcaaaggagaagtcc

SEQ ID NO: 100 - DNA, 56 bp, Artificial Sequence
  Synthetic
gctaaggctcatccattattcaaatatcgcaggtaccactggcaaaggagaagtcc

SEQ ID NO: 101 - DNA, 56 bp, Artificial Sequence
  Synthetic
gctaaggctcatccattattcaaatactatcggtaccactggcaaaggagaagtcc

SEQ ID NO: 102 - DNA, 56 bp, Artificial Sequence
  Synthetic
gctaaggctcatccattattcaaatactgtgggtaccactggcaaaggagaagtcc

SEQ ID NO: 103 - DNA, 56 bp, Artificial Sequence
  Synthetic
gctaaggctcatccattattcaaatactgcagccaccactggcaaaggagaagtcc

SEQ ID NO: 104 - DNA, 56 bp, Artificial Sequence
  Synthetic
gctaaggctcatccattattcaaatactgcaggtgccactggcaaaggagaagtcc

SEQ ID NO: 105 - DNA, 56 bp, Artificial Sequence
  Synthetic
gctaaggctcatccattattcaaatactgcaggtatcactggcaaaggagaagtcc

SEQ ID NO: 106 - DNA, 91 bp, Artificial Sequence; identical to SEQ ID NO: 96
  Synthetic

SEQ ID NO: 107 - DNA, 22 bp, Artificial Sequence
  Synthetic
caccatggagaccctcttgggc

SEQ ID NO: 108 - DNA, 67 bp, Artificial Sequence
  Synthetic
ctggttcctcttccaaatgtaggtatgtagcttcctcctgatgtgggcctcacagcacagaggtagg

SEQ ID NO: 109 - DNA, 66 bp, Artificial Sequence
  Synthetic
ctacatacctacatttggaagaggaaccagccttattgttcatccgtatatccagaaccctgaccc

SEQ ID NO: 110 - DNA, 22 bp, Artificial Sequence
  Synthetic
tcagctggaccacagccgcagc

SEQ ID NO: 111 - DNA, 24 bp, Artificial
  Synthetic
caccatgagcatcggcctcctgtg

SEQ ID NO: 112 - DNA, 40 bp, Artificial Sequence
  Synthetic
ctccccggtgttcccgacgtaactgctggcacagaagtac

SEQ ID NO: 113 - DNA, 44 bp, Artificial Sequence
  Synthetic
cagcagttacgtcgggaacaccggggagctgttttttggagaag

SEQ ID NO: 114 - DNA, 25 bp, Artificial Sequence
  Synthetic
ctagcctctggaatcctttctcttg

SEQ ID NO: 115 - DNA, 36 bp, Artificial Sequence
  Synthetic
taccatggagaccctcttgggcctgcttatcctttg

SEQ ID NO: 116 - DNA, 76 bp, Artificial Sequence
  Synthetic
cgccggcctgcttcagcaggctgaagttggtggctccggatccggaccgcttggcccggctggaccacagccgcag

SEQ ID NO: 117 - DNA, 61 bp, Artificial Sequence
  Synthetic
ctgcggctgtggtccagcggatccggagccaccaacttcagcctgctgaagcaggccggcg

SEQ ID NO: 118 - DNA, 63 bp, Artificial Sequence
  Synthetic
cctgctgaagcaggccggcgacgtggaggagaaccccggccccatgagcatcggcctcctgtg

SEQ ID NO: 119 - DNA, 44 bp, Artificial Sequence
  Synthetic
ttgaattctagcctctggaatcctttctcttgaccatagccatc

SEQ ID NO: 120 - protein, 9 aa, Artificial Sequence
  Synthetic
SLLMWITQC

SEQ ID NO: 121 - protein, 296 aa, Artificial Sequence
  Synthetic
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLTVL

SEQ ID NO: 122 - protein, 255 aa, Artificial Sequence
  Synthetic
KQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 123 - protein, 98 aa, Artificial Sequence
  Synthetic
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGN

SEQ ID NO: 124 - protein, 97 aa, Artificial Sequence
  Synthetic
KQEVTQIPAALSVPEGENLVLNCSFTDSAIYNLQWFRQDPGKGLTSLLLIQSSQREQTSGRLNASLDKSSGRSTLYIAASQPGDSATYLCAVRPTSG

SEQ ID NO: 125 - protein, 296 aa, Artificial Sequence
  Synthetic
NAGVTQTPKFQVLKTGQSMTLQCAQDMNHEYMSWYRQDPGMGLRLIHYSVXXXXTDQGEVPNGYNVSRSTTEDFPLRLLSAAPSQTSVYFCASSYXXXTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGLTVL

SEQ ID NO: 126 - protein, 255 aa, Artificial Sequence
  Synthetic
KQEVTQIPAALSVPEGENLVLNCSFTDSAIYNLQWFRQDPGKGLTSLLLIXXXQREQTSGRLNASLDKSSGRSTLYIAASQPGDSATYLCAVRXXXXGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 127 - protein, 98 aa, Artificial Sequence
  Synthetic
NAGVTQTPKFQVLKTGQSMTLQCAQDMNHEYMSWYRQDPGMGLRLIHYSVXXXXTDQGEVPNGYNVSRSTTEDFPLRLLSAAPSQTSVYFCASSYXXX

SEQ ID NO: 128 - protein, 97 aa, Artificial Sequence
  Synthetic
KQEVTQIPAALSVPEGENLVLNCSFTDSAIYNLQWFRQDPGKGLTSLLLIXXXQREQTSGRLNASLDKSSGRSTLYIAASQPGDSATYLCAVRXXXX

SEQ ID NO: 129 - protein, 287 aa, Artificial Sequence
  Synthetic
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 130 - protein, 94 aa, Artificial Sequence
  Synthetic
IAGITQAPTSQILAAGRRMTLRCTQDMRHNAMYWYRQDLGLGLRLIHYSNTAGTTGKGEVPDGYSVSRANTDDFPLTLASAVPSQTSVYFCASS

SEQ ID NO: 131 - protein, 287 aa, Artificial Sequence
  Synthetic
IAGITQAPTSQILAAGRRMTLRCTQDMRHNAMYWYRQDLGLGLRLIHYSNXXXXTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 132 - protein, 94 aa, Artificial Sequence
  Synthetic
IAGITQAPTSQILAAGRRMTLRCTQDMRHNAMYWYRQDLGLGLRLIHYSNXXXXTGKGEVPDGYSVSRANTDDFPLTLASAVPSQTSVYFCASS

SEQ ID NO: 133 - protein, 250 aa, Artificial Sequence
  Synthetic
GQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 134 - protein, 292 aa, Artificial Sequence
  Synthetic
NAGVTQTPKFQVLKTGQSMTLQCAQDMNHEYMSWYRQDPGMGLRLIHYSVAIGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 135 - protein, 94 aa, Artificial Sequence
  Synthetic
GQQLNQSPQSMFIQEGEDVSMNCTSSSIFNTWLWYKQDPGEGPVLLIALYKAGELTSNGRLTAQFGITRKDSFLNISASIPSDVGIYFCAGXXX

SEQ ID NO: 136 - protein, 95 aa, Artificial Sequence
  Synthetic
NAGVTQTPKFQVLKTGQSMTLQCAQDMNHEYMSWYRQDPGMGLRLIHYSVAIGITDQGEVPNGYNVSRSTTEDFPLRLLSAAPSQTSVYFCASXX

(19) United States
(12) Patent Application Publication
(10) Pub. No.: US 2010/0297093 A1
(43) Pub. Date: Nov. 25, 2010

(54) MODIFIED T CELL RECEPTORS AND RELATED MATERIALS AND METHODS

(75) Inventors: Paul F. Robbins, Potomac, MD (US); Steven A. Rosenberg, Potomac, MD (US); Richard A. Morgan, Columbia, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America, as represented by the Secretary, Department of Health and Human, Bethesda, MD (US)

(21) Appl. No.: 12/679,526

(22) PCT Filed: Sep. 23, 2008
(86) PCT No.: PCT/US08/77333

§ 371 Date: May 3, 2010

Related U.S. Application Data
(60) Provisional application No. 60/974,872, filed on Sep. 25, 2007.

(51) Int. Cl. A61K 35/26; G01N 33/53; C12N 5/10; C07K 14/00; C07H 21/04; A61P 35/00

(52) U.S. Cl. 424/93.71; 435/7.1; 435/325; 530/350; 536/23.1

(57) ABSTRACT

The invention is directed to a modified T cell receptor (TCR) comprising an amino acid sequence of a wild-type (WT) TCR with one or more amino acid substitutions in the CDR2 and/or CDR3 regions of the alpha and/or beta chains of the TCR, wherein the modified TCR, as compared to the WT TCR, (i) has an enhanced ability to recognize target cells when expressed by CD4⁺ T cells and/or CD8⁺ T cells and (ii) does not exhibit a decrease in antigen specificity when expressed by the CD4⁺ T cells and/or CD8⁺ T cells. Polypeptides, proteins, nucleic acids, recombinant expression vectors, host cells, populations of cells, antibodies, and pharmaceutical compositions related to the modified TCR also are part of the invention. Further, the invention is directed to methods of treating or preventing a disease in a host and methods of detecting a diseased cell in a host.

### CROSS-REFERENCE TO RELATED APPLICATIONS

This patent application claims the benefit of U.S. Provisional Patent Application No. 60/974,872, filed Sep. 25, 2007, which is incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 82,115 Byte ASCII (Text) file named “703526ST25.TXT,” created on Sep. 23, 2008.

### BACKGROUND OF THE INVENTION

In an ongoing adoptive transfer clinical trial, cancer patients received autologous peripheral blood mononuclear cells (PBMC) that were transduced with nucleic acids encoding a T cell receptor (TCR) specific for the melanoma antigen MART-1. Thus far, two out of 17 patients have demonstrated an objective clinical response (Morgan et al., Sciencexpress, e-publication Aug. 31, 2006). The results of this clinical trial demonstrate that normal autologous T lymphocytes, transduced ex vivo with anti-cancer antigen TCR genes and reinfused in cancer patients, can persist and express the transgene long term in vivo and mediate durable regression of large established tumors. However, approaches to increase the expression and function of the transgene are still needed.

### BRIEF SUMMARY OF THE INVENTION

The invention provides proteins, namely modified T cell receptors (TCRs), each of which comprises an amino acid sequence of a wild-type TCR with one or more amino acid substitutions in the CDR2 and/or CDR3 regions of the alpha and/or beta chains of the TCR, wherein the modified TCR, as compared to the WT TCR, (i) has an enhanced ability to recognize target cells when expressed by CD4⁺ T cells and/or CD8⁺ T cells, and (ii) does not exhibit a decrease in antigen specificity when expressed by the CD4⁺ T cells and/or CD8⁺ T cells. The invention also provides related polypeptides and proteins, as well as related nucleic acids, recombinant expression vectors, host cells, and populations of cells. Further provided by the invention are antibodies, or an antigen binding portion thereof, and pharmaceutical compositions relating to the modified TCRs of the invention.

Also provided by the invention is a method of treating or preventing a disease in a host. The method comprises administering to the host a pharmaceutical composition comprising a population of cells comprising the inventive host cells in an amount effective to treat or prevent the disease in the host.

Further, the invention provides a method of detecting a diseased cell in a host, wherein the diseased cell expresses an antigen characteristic of a disease. The method comprises (a) contacting a sample comprising cells of the host with an inventive modified TCR, thereby forming a complex between the modified TCR and the antigen, and (b) detecting the complex, wherein detection of the complex is indicative of a diseased cell in the host.

### BRIEF DESCRIPTION OF THE SEVERAL VIEWS OF THE DRAWINGS

FIG. 1A represents a graph of the IFN-γ (pg/ml) release by anti-CD3-stimulated CD8+ T cells transfected with RNA encoding TCR constructs containing substitution of 1G4 CDR3α residues along with the 1G4 WT P chain. Transfected T cells are incubated overnight with T2 cells pulsed with 1 μM (black bars), 10 nM (diagonal bars), or 0.1 nM (dotted bars) NY-ESO-1 peptide or 1 μM control peptide (gp100:154-162; checkered bars). IFN-γ release is measured the following day.

FIGS. 1B and 1C each represent a graph of the IFN-γ (pg/ml) release by anti-CD3-stimulated CD8+ T cells (FIG. 1B) or CD4+ T cells (FIG. 1C) transfected with RNA encoding TCR constructs containing substitution of 1G4 CDR3a residues along with the 1G4 WT β chain. Transfected T cells are incubated overnight with tumor cells: 397A2 (A2+/ESO+; black bars), 624.38 (A2+/ESO+; diagonal bars), 1300 (A2+/ESO+; vertical striped bars), MDA435S-A2 (A2+/ESO+; dotted bars), 526 (A2+/ESO−; horizontal striped bars), and 888A2 (A2+/ESO−; white bars). IFN-γ release is measured the following day.

FIG. 2A represents a graph of the IFN-γ release by anti-CD3-stimulated CD8+ T cells transfected with RNA encoding TCR constructs containing substitution of 1G4 CDR3p residues along with the 1G4 WT α chain. Transfected T cells are incubated overnight with T2 cells pulsed with 1 μM (black bars), 10 nM (diagonal bars), 0.1 nM (dotted bars) NY-ESO-1 peptide or 1 μM control peptide (gp100:154-162; checkered bars). IFN-γ release is measured the following day.

FIG. 2B represents a graph of the IFN-γ release by anti-CD3-stimulated CD8+ T cells transfected with RNA encoding TCR constructs containing substitution of 1G4 CDR213 residues along with the 1G4 WT α chain. Transfected T cells are incubated overnight with T2 cells pulsed with 100 nM (black bars), 10 nM (diagonal bars), 1 nM (dotted bars), or 0.1 nM (checkered bars) NY-ESO-1 peptide or 100 nM control peptide (gp100:154-162; white bars). IFN-γ release is measured the following day.

FIGS. 2C and 2D each represent a graph of the IFN-γ (pg/ml) release by anti-CD3-stimulated CD8+ T cells (FIG. 2C) or CD4+ T cells (FIG. 2D) transfected with RNA encoding TCR constructs containing substitution of 1G4 CDR2β residues along with the 1G4 WT α chain. Transfected T cells are incubated overnight with tumor cells: 624.38 (A2+/ESO+; black bars), 1363 (A2+/ESO+; diagonal bars), A375 (A2+/ESO+; dotted bars), SK23 (A2+/ESO−; checkered bars), and 526 (A2+/ESO−; white bars). IFN-γ release is measured the following day.

FIG. 2D represents a graph of the IFN-γ (pg/ml) release by anti-CD3-stimulated CD4+ T cells transfected with RNA encoding TCR constructs containing substitution of 1G4 CDR2β residues along with the 1G4 WT α chain. Transfected T cells are incubated overnight with tumor cells: 624.38 (A2+/ESO+; black bars), 1363 (A2+/ESO+; diagonal bars), A375 (A2+/ESO+; dotted bars), SK23 (A2+/ESO−; checkered bars), and 526 (A2+/ESO−; white bars). IFN-γ release is measured the following day.

FIGS. 3A and 3B each represent a graph of the IFN-γ (pg/ml) release by CD8+ T cells (FIG. 3A) and CD4+ T cells (FIG. 3B) transfected with RNA encoding TCR constructs containing substitution of DMF5 CDR2β residues along with the DMF5 WT α chain. Transfected T cells are incubated overnight with tumor cells: 624 (A2+/MART+; black bars), 526 (A2+/MART+; diagonal bars), 1359-A2 (A2+/MART+; dotted bars), 1363 (A2+/MART+; striped bars), and A375 (A2+/MART−; white bars). IFN-γ release is measure the following day.

FIG. 3B represents a graph of the IFN-γ (pg/ml) release by CD4+ T cells transfected with RNA encoding TCR constructs containing substitution of DMF5 CDR213 residues along with the DMF5 WT α chain. Transfected T cells are incubated overnight with tumor cells: 624 (A2+/MART+; black bars), 526 (A2+/MART+; diagonal bars), 1359-A2 (A2+/MART+; dotted bars), 1363 (A2+/MART+; striped bars), and A375 (A2+/MART−; white bars). IFN-γ release is measured the following day.

FIGS. 4A and 4B each represent a graph of the IFN-γ (pg/ml) release by CD8+ T cells (FIG. 4A) or CD4+ T cells (FIG. 4B) transfected with RNA encoding TCR constructs containing substitution of DMF4 CDR2β residues along with the DMF4 WT α chain. Transfected T cells are incubated overnight with tumor cells: 397-A2 (A2+/MART+; black bars), 624.38 (A2+/MART+; diagonal bars), 1300 (A2+/MART+; dotted bars), SK23 (A2+/MART+; striped bars), A375 (A2+/MART−; checkered bars), and 397-A24 (A2⁻/MART+; white bars). IFN-γ release is measured the following day.

FIGS. 5A and 5B each represent a graph of the IFN-γ (pg/ml) release by CD8+ T cells (FIG. 5A) or CD4+ T cells (FIG. 5B) transduced with RNA encoding TCR constructs containing substitution of 1G4 CDR2β or CDR3α residues along with the 1G4 WT α or β chain. Transfected T cells are incubated overnight alone (white bars) or with tumor cells: 624.38 (A2+/ESO+; black bars), H1299A2 (A2+/ESO+; diagonal bars), and 1300 (A2+/ESO+; checkered bars), 2661R (A2+/ESO−; striped bars). IFN-γ release is measured the following day.

FIGS. 6A and 6B each represent a panel of graphs of the % specific lysis of target cells (624.38 (left—A2+/ESO+); A375 (middle—A2+/ESO+), and 2661R (right—ESO−)) by CD8+ T cells (FIG. 6A) or CD4+ T cells (FIG. 6B) transfected with 1G4 WT and variant TCRs (WT (diamonds); β51:AI (squares); α95:LL (triangles); α95:LY (circles); and GFP (X's)) as determined by a standard 4 hour ⁵¹Cr release assay.

FIG. 7A represents a graph of the % specific lysis of target cells (624.38) by CD8+ T cells transfected with DMF4 WT or variant TCR thereof (left panel) or DMF5 WT or variant TCR thereof (right panel) vs. Effector Cell:Target Cell (E:T), as determined by a standard 4 hour ⁵¹Cr release assay. DMF4 WT (left panel—diamonds); DMF4 β51:A (left panel—squares); GFP (left and right panels—triangles); DMF5 WT (right panel—diamonds); DMF5 β54:A (right panel—squares)).

FIG. 7B represents a graph of the % specific lysis of target cells (2661R) by CD8+ T cells transfected with DMF4 WT or variant TCR thereof (left panel) or DMF5 WT or variant TCR thereof (right panel) vs. Effector Cell:Target Cell (E:T), as determined by a standard 4 hour ⁵¹Cr release assay. DMF4 WT (left panel—diamonds); DMF4 β51:A (left panel—squares); GFP (left and right panels—triangles); DMF5 WT (right panel—diamonds); DMF5 β54:A (right panel—squares)).

FIG. 7C represents a graph of the % specific lysis of target cells (624.38) by CD4+ T cells transfected with DMF4 WT or variant TCR thereof (left panel) or DMF5 WT or variant TCR thereof (right panel) vs. Effector Cell:Target Cell (E:T), as determined by a standard 4 hour ⁵¹Cr release assay. DMF4 WT (left panel—diamonds); DMF4 β51:A (left panel—squares); GFP (left and right panels—triangles); DMF5 WT (right panel—diamonds); DMF5 β54:A (right panel—squares)).

FIG. 7D represents a graph of the % specific lysis of target cells (2661R) by CD4+ T cells transfected with DMF4 WT or variant TCR thereof (left panel) or DMF5 WT or variant TCR thereof (right panel) vs. Effector Cell:Target Cell (E:T), as determined by a standard 4 hour ⁵¹Cr release assay. DMF4 WT (left panel—diamonds); DMF4 β51:A (left panel—squares); GFP (left and right panels—triangles); DMF5 WT (right panel—diamonds); DMF5 β54:A (right panel—squares)).

FIG. 8A represents a graph of the IFN-γ (pg/ml) release by CD8+ T cells transfected with 1G4 WT beta chain and 1G4 WT α chain (diamonds) or CDR3α TCR variants (T95L (squares); S96L (X's); S96Y (ticks); T95L/S96L (triangles); and T95L/S96Y (circles) vs. peptide concentration (nM).

FIG. 8B represents a graph of the IFN-γ (pg/ml) release by CD8+ T cells transfected with 1G4 WT α chain (diamonds) or CDR2β TCR variants (G51A (squares); A52I (triangles); G51A/A52I (circles); or with GFP (Xs); vs. peptide concentration (nM).

FIG. 8C represents a graph of the IFN-γ (pg/ml) released by CD4+ T cells transfected with 1G4 WT α chain (diamonds) or CDR3α TCR variants (T95L (squares); S96L (X's); S96Y (ticks); T95L/S96L (triangles); and T95L/S96Y (circles) vs. peptide concentration (nM).

FIG. 8D represents a graph of the IFN-γ (pg/ml) release by CD4+ T cells transfected with 1G4 WT α chain (diamonds) or CDR20TCR variants (G51A (squares); A52I (triangles); G51A/A52I (circles); or with GFP (Xs); vs. peptide concentration (nM).

FIGS. 9A and 9B each represents a graph of the IFN-γ (pg/ml) release by CD8+ T cells (FIG. 9A) or CD4+ T cells (FIG. 9B) transduced with 1G4 WT chains or 1G4 variant TCRs. Transfected T cells are incubated overnight with tumor cells: 624.38 (A2+/ESO+; black bars), 1300 (A2+/ESO+; diagonal bars), H1299A2 (A2+/ESO+; checkered bars), SK23 (A2+/ESO−; striped bars), and 2661R (A2+/ESO−; white bars). IFN-γ release is measured the following day.

### DETAILED DESCRIPTION OF THE INVENTION

The invention provides proteins, namely modified T cell receptors (TCRs), each of which comprises an amino acid sequence of a wild-type (WT) TCR with one or more amino acid substitutions, wherein the modified TCR, as compared to the WT TCR, (i) has an enhanced ability to recognize target cells when expressed by CD4⁺ T cells and/or CD8⁺ T cells and (ii) does not exhibit a decrease in antigen specificity when expressed by the CD4⁺ T cells and/or CD8⁺ T cells.

The term “wild-type” as used herein refers to a TCR which is naturally expressed by a T cell of a host, e.g., a TCR which is endogenous to a T cell of a host. Nucleic acids encoding wild-type TCRs are known in the art and can be obtained from the GenBank database of the National Center for Biotechnology Information (NCBI). For example, wild-type TCR nucleic acid sequences are available as GenBank Accession Nos. NG_{—}001333, NG_{—}000016, NG_{—}001337, NG_{—}001332, NG_{—}001336, AF043179, HSJ004872, M13863, Z81026, AF397440, AY124793, and the like. Also, nucleic acids encoding wild-type TCRs can be obtained by methods known in the art, such as a PCR-based method.

The modified TCR of the invention is characterized by one or more enhanced biological properties when expressed in T cells. Specifically, the modified TCR, when compared to the corresponding WT TCR, has an enhanced ability to recognize target cells when expressed by CD4⁺ T cells and/or CD8⁺ T cells. Also, the modified TCR does not exhibit a decrease in antigen specificity when expressed by the CD4⁺ T cells and/or CD8⁺ T cells. The term “target cells” as used herein refers to cells which express and present, by way of an MHC molecule, the antigen which is specifically recognized by the modified TCR. The phrase “recognize target cells” as used herein refers to the ability of the modified TCR to immunologically recognize (e.g., specifically bind to) an antigen which is expressed and presented by a target cell. The term “enhanced” as used herein means that the modified TCR of the invention consistently exhibits an increase in ability to recognize antigen expressed and presented by target cells, as compared to its WT counterpart. Preferably, the modified TCR of the invention consistently exhibits at least a 0.5×, 2×, or 5× increase in the ability to recognize antigen expressed and presented by target cells as compared to its WT counterpart. More preferably, the modified TCRs of the invention recognize antigen at least ten times better than their WT counterparts.

The modified TCR of the invention exhibits an enhanced ability to recognize target cells without exhibiting a decrease in antigen specificity when expressed by CD4⁺ T cells and/or CD8⁺ T cells. In this respect, the modified TCR is said to retain the antigen specificity of the counterpart WT TCR, e.g., recognizes only the antigen(s) recognized by the WT TCR and does not recognize antigen(s) that are not recognized by the WT TCR.

Methods of testing a TCR for the ability to recognize target cells and for antigen specificity are known in the art. For instance, Clay et al., J. Immunol., 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, TCR function can be evaluated by measurement of cellular cytoxicity, as described in Zhao et al., J. Immunol., 174: 4415-4423 (2005). Methods of testing a modified TCR for the ability to recognize target cells and for antigen specificity are described herein as Examples 1 to 12.

The modified TCR of the invention can have antigen specificity for any antigen, provided that its WT counterpart recognizes the antigen. The phrase “have antigen specificity” as used herein means that the modified TCR can specifically bind to and immunologically recognize an antigen, such that binding of the TCR to the antigen elicits an immune response.

Preferably, the modified TCR of the invention has antigen specificity for an antigen which is characteristic of a disease. The disease can be any disease involving an antigen, as discussed herein, e.g., an infectious disease, an autoimmune disease, or a cancer. The antigen could be, for example, a viral antigen, a bacterial antigen, a cancer antigen, etc.

More preferably, the modified TCR of the invention has antigen specificity for a cancer antigen. The term “cancer antigen” as used herein refers to any molecule (e.g., protein, peptide, lipid, carbohydrate, etc.) solely or predominantly expressed or over-expressed by a tumor cell or cancer cell, such that the antigen is associated with the tumor or cancer. The cancer antigen additionally can be expressed by normal, non-tumor, or non-cancerous cells. However, in such a situation, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen additionally can be expressed by cells of a different state of development or maturation. For instance, the cancer antigen can be additionally expressed by cells of the embryonic or fetal stage, which cells are not normally found in an adult host. Alternatively, the cancer antigen additionally can be expressed by stem cells or precursor cells, which cells are not normally found in an adult host. Another group of cancer antigens are represented by the differentiation antigens that are expressed in only a limited set of tissues in the adult, such as the melanocytes differentiation antigens, whose expression is limited to normal melanocytes. Although it is not known why these molecules elicit immune responses, the limited expression pattern of these proteins may allow these molecules to be recognized by the immune system.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. In a preferred embodiment of the invention, the cancer antigen is a melanoma cancer antigen. In a more preferred embodiment, the cancer antigen is selected from the group consisting of NY-ESO-1, MART-1, gp100, p53, TRP-1, TRP-2, and tyrosinase. In a most preferred embodiment, the cancer antigen is NY-ESO-1 or MART-1.

With respect to the inventive modified TCR, the amino acid substitution(s) can be located in any part of the amino acid sequence of the TCR. Preferably, the amino acid substitutions are located within the amino acid sequence of the complementary determining region (CDR) of the TCR, which is known in the art. These regions have been defined by elucidation of X-ray crystallographic structures, as well as sequence comparisons which have revealed the presence of regions of high diversity encoded in germline sequences, in the case of CDR1 and CDF2 regions, as well as recombinational diversity, in the case of CDR3 region (Lefranc et al., Nucl. Acids Res., 27, 209-212 (1999)). Preferably, the one or more, e.g., one, two, or three, amino acid substitutions are located in the amino acid sequence of a CDR2 or CDR3 of the TCR (e.g., in the CDR2 region of the beta chain of the TCR). More preferably, the amino acid substitutions are located in the amino acid sequence of a CDR2, e.g., CDR2 of an α chain of a TCR or a β chain of a TCR. Most preferably, the amino acid substitutions are located in the CDR2 or CDR3 of an α or β chain of a TCR.

The invention provides a modified TCR comprising two polypeptides (i.e., polypeptide chains), such as an α chain of a TCR, a β chain of a TCR, a γ chain of a TCR, a δ chain of a TCR, or a combination thereof. The amino acid substitutions of the inventive modified TCRs can be located in the amino acid sequence of either or both polypeptide chains which constitute the TCR.

The amino acid substitutions of the inventive modified TCR are preferably conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The polypeptide chains of the inventive modified TCR can comprise any amino acid sequence, provided that the modified TCR, as compared to the WT TCR, (i) has an enhanced ability to recognize antigen when expressed by CD4⁺ T cells and (ii) does not exhibit a decrease in antigen specificity when expressed by CD8⁺ T cells.

In a preferred embodiment of the invention, the modified TCR comprises the amino acid sequence of SEQ ID NO: 1 or 2 with no more than seven, e.g., 1, 2, 3, 4, 5, 6, or 7, amino acid substitutions. In a more preferred embodiment of the invention, the modified TCR comprises the amino acid sequence of SEQ ID NO: 7 or 8, each of which is an amino acid sequence of a variable region of a beta chain or an alpha chain of a modified TCR with amino acid substitutions in the CDR2 and/or CDR3, which TCR recognizes the cancer antigen NY-ESO-1. In a most preferred embodiment of the invention, the modified TCR comprises the amino acid sequence of SEQ ID NO: 5 or 6, each of which is the amino acid sequence of a full-length beta chain or alpha chain comprising a constant region and a variable region in which up to seven amino acids in the CDR2 and/or CDR3 have been modified.

In another preferred embodiment, the modified TCR comprises the amino acid sequence of SEQ ID NO: 9 or 10 with up to four amino acid substitutions. In a more preferred embodiment, the modified TCR comprises an amino acid sequence of SEQ ID NO: 12, which is an amino acid sequence of a variable region of a beta chain of a modified TCR with amino acid substitutions in the CDR2, which TCR recognizes the cancer antigen MART-1. In a most preferred embodiment of the invention, the modified TCR comprises the amino acid sequence of SEQ ID NO: 11, which is the amino acid sequence of a full-length beta chain comprising a constant region and a variable region in which up to four amino acids in the CDR2 have been modified.

Preferably, the modified TCR comprises an amino acid sequence selected from Group I, which consists of:
(i) SEQ ID NO: 7, wherein Xaa at positions 70-72 and 115-117 are wild-type amino acids, wherein Xaa at position 73 is Thr;
(ii) SEQ ID NO: 7, wherein Xaa at positions 70 and 115-117 are wild-type amino acids, wherein Xaa at positions 71-73 are Ile, Gln, and Ile, respectively;
(iii) SEQ ID NO: 7, wherein Xaa at positions 70, 72, and 115-117 are wild-type amino acids, wherein Xaa at positions 71 and 73 are Ile and Thr, respectively;
(iv) SEQ ID NO: 7, wherein Xaa at positions 70, 71, and 115-117 are wild-type amino acids, wherein Xaa at positions 72 and 73 are Gln and Thr, respectively;
(v) SEQ ID NO: 7, wherein Xaa at positions 73 and 115-117 are wild-type amino acids, wherein Xaa at positions 70-72 are Ala, Ile, Gln, respectively;
(vi) SEQ ID NO: 7, wherein Xaa at positions 72 and 115-117 are wild-type amino acids, wherein Xaa at positions 70, 71, and 73 are Ala, Ile, and Thr, respectively;
(vii) SEQ ID NO: 7, wherein Xaa at positions 70 and 115-117 are wild-type amino acids, wherein Xaa at positions 71-73 are Ile, Gln, and Thr, respectively;
(viii) SEQ ID NO: 7, wherein Xaa at positions 115-117 are wild-type amino acids, wherein Xaa at positions 70-73 are Ala, Ile, Gln, and Thr respectively;
(ix) SEQ ID NO: 7, wherein Xaa at positions 70-73, 115, and 116 are wild-type amino acids, wherein Xaa at position 117 is Leu;
(x) SEQ ID NO: 8, wherein Xaa at positions 113-116 are wild-type amino acids, wherein Xaa at positions 70-72 are Thr, Pro, and Trp, respectively;
(xi) SEQ ID NO: 8, wherein Xaa at positions 71, 72, and 113-116 are wild-type amino acids, wherein Xaa at position 70 is Pro;
(xii) SEQ ID NO: 8, wherein Xaa at positions 71, 72, and 113-116 are wild-type amino acids, wherein Xaa at position 70 is Thr;
(xiii) SEQ ID NO: 8, wherein Xaa at positions 70, 72, and 113-116 are wild-type amino acids, wherein Xaa at position 71 is Phe;
(xiv) SEQ ID NO: 8, wherein Xaa at positions 70, 72, and 113-116 are wild-type amino acids, wherein Xaa at position 71 is Pro;
(xv) SEQ ID NO: 8, wherein Xaa at positions 70, 71, and 113-116 are wild-type amino acids, wherein Xaa at position 72 is Trp;
(xvi) SEQ ID NO: 8, wherein Xaa at positions 72 and 113-116 are wild-type amino acids, wherein Xaa at positions 70 and 71 are Pro and Phe, respectively;
(xvii) SEQ ID NO: 8, wherein Xaa at positions 71 and 113-116 are wild-type amino acids, wherein Xaa at positions 70 and 72 are Pro and Trp;
(xviii) SEQ ID NO: 8, wherein Xaa at positions 70 and 113-116 are wild-type amino acids, wherein Xaa at positions 71 and 72 are Phe and Trp;
(xix) SEQ ID NO: 8, wherein Xaa at positions 72 and 113-116 are wild-type amino acids, wherein each Xaa at positions 70 and 71 is Pro, respectively;
(xx) SEQ ID NO: 8, wherein Xaa at positions 72 and 113-116 are wild-type amino acids, wherein Xaa at positions 70 and 71 is Thr and Pro, respectively;
(xxi) SEQ ID NO: 8, wherein Xaa at positions 71 and 113-116 are wild-type amino acids, wherein Xaa at positions 70 and 72 are Thr and Trp, respectively;
(xxii) SEQ ID NO: 8, wherein Xaa at positions 70 and 113-116 are wildtype amino acids, wherein Xaa at positions 71 and 72 are Pro and Trp, respectively;
(xxiii) SEQ ID NO: 8, wherein Xaa at positions 72 and 113-116 are wild-type amino acids, wherein Xaa at positions 70 and 71 are Thr and Phe, respectively;
(xxiv) SEQ ID NO: 8, wherein Xaa at positions 113-116 are wild-type amino acids, wherein Xaa at positions 70 to 72 are Pro, Phe, and Trp, respectively;
(xxv) SEQ ID NO: 8, wherein Xaa at positions 70-72, 113, and 116 are wild-type amino acids, wherein Xaa at positions 114 and 115 are Leu and Tyr, respectively;
(xxvi) SEQ ID NO: 8, wherein Xaa at positions 70-72, 113, 115, and 116 are wild-type amino acids, wherein Xaa at position 114 is Ala;
(xxvii) SEQ ID NO: 8, wherein Xaa at positions 70-72, 113, 115, and 116 are wild-type amino acids, wherein Xaa at position 114 is Leu;
(xxviii) SEQ ID NO: 8, wherein Xaa at positions 70-72, 113, 115, and 116 are wild-type amino acids, wherein Xaa at position 114 is Glu;
(xxix) SEQ ID NO: 8, wherein Xaa at positions 70-72, 113, 114, and 116 are wild-type amino acids, wherein Xaa at position 115 is Ala;
(xxx) SEQ ID NO: 8, wherein Xaa at positions 70-72, 113, 114, and 116 are wild-type amino acids, wherein Xaa at position 115 is Leu;
(xxxi) SEQ ID NO: 8, wherein Xaa at positions 70-72, 113, 114, and 116 are wild-type amino acids, wherein Xaa at position 115 is Tyr;
(xxxii) SEQ ID NO: 8, wherein Xaa at positions 70-72, 113, 114, and 116 are wild-type amino acids, wherein Xaa at position 115 is Glu;
(xxxiii) SEQ ID NO: 8, wherein Xaa at positions 70-72, 113, 114, and 116 are wild-type amino acids, wherein Xaa at position 115 is Lys;
(xxxiv) SEQ ID NO: 8, wherein Xaa at positions 70-72 and 113 are wild-type amino acids, wherein Xaa at positions 114-116 are Leu, Leu, and Asp, respectively;
(xxxv) SEQ ID NO: 8, wherein Xaa at positions 70-72, 113, and 116 are wild-type amino acids, wherein each Xaa at positions 114 and 115 are Leu;
(xxxvi) SEQ ID NO: 8, wherein Xaa at positions 70-72, 113, and 116 are wild-type amino acids, wherein Xaa at positions 114 and 115 are Glu and Tyr, respectively;
(xxxvii) SEQ ID NO: 8, wherein Xaa at positions 70-72, 115, and 116 are wild-type amino acids, wherein Xaa at positions 113 and 114 are Leu and Pro, respectively;
(xxxviii) SEQ ID NO: 12, wherein Xaa at positions 71 to 73 are wild-type amino acids, wherein Xaa at position 70 is Val; and
(xxxix) SEQ ID NO: 12, wherein Xaa at positions 70, 72, and 73 are wild-type amino acids, wherein Xaa at position 71 is Met;
or a combination thereof;
wherein the wild-type amino acids of SEQ ID NO: 7 at positions 70-73 and 115-117 are Gly, Ala, Gly, Ile, Val, Gly, and Asn, respectively,
wherein the wild-type amino acids of SEQ ID NO: 8 at positions 70-72 and 113-116 are Gln, Ser, Ser, Pro, Thr, Ser, and Gly, respectively,
wherein the wild-type amino acids of SEQ ID NO: 12 at positions 70-73 are Thr, Ala, Gly, and Thr, respectively.

Each of the amino acid sequences of Group I comprise a modified amino acid sequence as compared to the corresponding WT sequence, since each sequence comprises one or more AAS (amino acid substitution). For instance, the amino acid sequence of (i) is modified in comparison to SEQ ID NO: 3, which is the WT variable region of the beta chain of the 1G4 TCR. The amino acid sequence of (i) which differs from SEQ ID NO: 3 is the sequence GAGT (SEQ ID NO: 18), which is located within CDR2 of the beta chain. A summary of each amino acid sequence of Group I with respect to its corresponding WT amino acid sequence and the amino acid sequence which differs therefrom is set forth below in Table 1:

TABLE 1

| | | | | | Position of Amino | | |
|---|---|---|---|---|---|---|---|
| No. | | | | | Acids of Immature | | SEQ ID NO: |
| within | | TCR | CDR with | | Sequence (of Mature | Modified | of Modified |
| Group I | WT TCR | Chain | AAS | WT Sequence | Sequence) | Sequence | Sequence |
| i | 1G4 | beta | CDR2 | GAGI (SEQ ID NO: 17) | 70-73 (51-54) | GAGT | 18 |
| ii | 1G4 | beta | CDR2 | GAGI (SEQ ID NO: 17) | 70-73 (51-54) | GIQI | 19 |
| iii | 1G4 | beta | CDR2 | GAGI (SEQ ID NO: 17) | 70-73 (51-54) | GIGT | 20 |
| iv | 1G4 | beta | CDR2 | GAGI (SEQ ID NO: 17) | 70-73 (51-54) | GAQT | 21 |
| v | 1G4 | beta | CDR2 | GAGI (SEQ ID NO: 17) | 70-73 (51-54) | AIQI | 22 |
| vi | 1G4 | beta | CDR2 | GAGI (SEQ ID NO: 17) | 70-73 (51-54) | AIGT | 23 |
| vii | 1G4 | beta | CDR2 | GAGI (SEQ ID NO: 17) | 70-73 (51-54) | GIQT | 24 |
| viii | 1G4 | beta | CDR2 | GAGI (SEQ ID NO: 17) | 70-73 (51-54) | AIQT | 25 |
| ix | 1G4 | beta | CDR3 | VGN | 115-117 (96-98) | VGL | |
| x | 1G4 | alpha | CDR2 | QSS | 70-72 (51-53) | TPW | |
| xi | 1G4 | alpha | CDR2 | QSS | 70-72 (51-53) | PSS | |
| xii | 1G4 | alpha | CDR2 | QSS | 70-72 (51-53) | TSS | |
| xiii | 1G4 | alpha | CDR2 | QSS | 70-72 (51-53) | QFS | |
| xiv | 1G4 | alpha | CDR2 | QSS | 70-72 (51-53) | QPS | |
| xv | 1G4 | alpha | CDR2 | QSS | 70-72 (51-53) | QSW | |
| xvi | 1G4 | alpha | CDR2 | QSS | 70-72 (51-53) | PFS | |
| xvii | 1G4 | alpha | CDR2 | QSS | 70-72 (51-53) | PSW | |
| xviii | 1G4 | alpha | CDR2 | QSS | 70-72 (51-53) | QFW | |
| xix | 1G4 | alpha | CDR2 | QSS | 70-72 (51-53) | PPS | |
| xx | 1G4 | alpha | CDR2 | QSS | 70-72 (51-53) | TPS | |
| xxi | 1G4 | alpha | CDR2 | QSS | 70-72 (51-53) | TSW | |
| xxii | 1G4 | alpha | CDR2 | QSS | 70-72 (51-53) | QPW | |
| xxiii | 1G4 | alpha | CDR2 | QSS | 70-72 (51-53) | TFS | |
| xxiv | 1G4 | alpha | CDR2 | QSS | 70-72 (51-53) | PFW | |
| xxv | 1G4 | alpha | CDR3 | PTSG (SEQ ID NO: 26) | 113-116 (94-97) | PLYG | 27 |
| xxvi | 1G4 | alpha | CDR3 | PTSG (SEQ ID NO: 26) | 113-116 (94-97) | PASG | 28 |
| xvii | 1G4 | alpha | CDR3 | PTSG (SEQ ID NO: 26) | 113-116 (94-97) | PLSG | 29 |
| xviii | 1G4 | alpha | CDR3 | PTSG (SEQ ID NO: 26) | 113-116 (94-97) | PESG | 30 |
| xxix | 1G4 | alpha | CDR3 | PTSG (SEQ ID NO: 26) | 113-116 (94-97) | PTAG | 31 |
| xxx | 1G4 | alpha | CDR3 | PTSG (SEQ ID NO: 26) | 113-116 (94-97) | PTLG | 32 |
| xxxi | 1G4 | alpha | CDR3 | PTSG (SEQ ID NO: 26) | 113-116 (94-97) | PTYG | 33 |
| xxxii | 1G4 | alpha | CDR3 | PTSG (SEQ ID NO: 26) | 113-116 (94-97) | PTEG | 34 |
| xxxiii | 1G4 | alpha | CDR3 | PTSG (SEQ ID NO: 26) | 113-116 (94-97) | PTKG | 35 |
| xxxiv | 1G4 | alpha | CDR3 | PTSG (SEQ ID NO: 26) | 113-116 (94-97) | PLLD | 36 |
| xxxv | 1G4 | alpha | CDR3 | PTSG (SEQ ID NO: 26) | 113-116 (94-97) | PLLG | 37 |
| xxxvi | 1G4 | alpha | CDR3 | PTSG (SEQ ID NO: 26) | 113-116 (94-97) | PEYG | 38 |
| xxxvii | 1G4 | alpha | CDR3 | PTSG (SEQ ID NO: 26) | 113-116 (94-97) | LPSG | 39 |
| xxxviii | DMF5 (F5) | beta | CDR2 | TAGT (SEQ ID NO: 40) | 70-73 (51-54) | VAGT | 41 |
| xxxix | F5 | beta | CDR2 | TAGT (SEQ ID NO: 40) | 70-73 (51-54) | TMGT | 42 |

In a preferred embodiment of the invention, the modified TCR comprises an amino acid sequence selected from Group II, which consists of:
(i) SEQ ID NO: 5, wherein Xaa at positions 70-72 and 115-117 are wild-type amino acids, wherein Xaa at position 73 is Thr;
(ii) SEQ ID NO: 5, wherein Xaa at positions 70 and 115-117 are wild-type amino acids, wherein Xaa at positions 71-73 are Ile, Gln, and Ile, respectively;
(iii) SEQ ID NO: 5, wherein Xaa at positions 70, 72, and 115-117 are wild-type amino acids, wherein Xaa at positions 71 and 73 are Ile and Thr, respectively;
(iv) SEQ ID NO: 5, wherein Xaa at positions 70, 71, and 115-117 are wild-type amino acids, wherein Xaa at positions 72 and 73 are Gln and Thr, respectively;
(v) SEQ ID NO: 5, wherein Xaa at positions 73 and 115-117 are wild-type amino acids, wherein Xaa at positions 70-72 are Ala, Ile, Gln, respectively;
(vi) SEQ ID NO: 5, wherein Xaa at positions 72 and 115-117 are wild-type amino acids, wherein Xaa at positions 70, 71, and 73 are Ala, Ile, and Thr, respectively;
(vii) SEQ ID NO: 5, wherein Xaa at positions 70 and 115-117 are wild-type amino acids, wherein Xaa at positions 71-73 are Ile, Gln, and Thr, respectively;
(viii) SEQ ID NO: 5, wherein Xaa at positions 115-117 are wild-type amino acids, wherein Xaa at positions 70-73 are Ala, Ile, Gln, and Thr respectively;
(ix) SEQ ID NO: 5, wherein Xaa at positions 70-73, 115, and 116 are wild-type amino acids, wherein Xaa at position 117 is Leu;
(x) SEQ ID NO: 6, wherein Xaa at positions 113-116 are wild-type amino acids, wherein Xaa at positions 70-72 are Thr, Pro, and Trp, respectively;
(xi) SEQ ID NO: 6, wherein Xaa at positions 71, 72, and 113-116 are wild-type amino acids, wherein Xaa at position 70 is Pro;
(xii) SEQ ID NO: 6, wherein Xaa at positions 71, 72, and 113-116 are wild-type amino acids, wherein Xaa at position 70 is Thr;
(xiii) SEQ ID NO: 6, wherein Xaa at positions 70, 72, and 113-116 are wild-type amino acids, wherein Xaa at position 71 is Phe;
(xiv) SEQ ID NO: 6, wherein Xaa at positions 70, 72, and 113-116 are wild-type amino acids, wherein Xaa at position 71 is Pro;
(xv) SEQ ID NO: 6, wherein Xaa at positions 70, 71, and 113-116 are wild-type amino acids, wherein Xaa at position 72 is Trp;
(xvi) SEQ ID NO: 6, wherein Xaa at positions 72 and 113-116 are wild-type amino acids, wherein Xaa at positions 70 and 71 are Pro and Phe, respectively;
(xvii) SEQ ID NO: 6, wherein Xaa at positions 71 and 113-116 are wild-type amino acids, wherein Xaa at positions 70 and 72 are Pro and Trp;
(xviii) SEQ ID NO: 6, wherein Xaa at positions 70 and 113-116 are wild-type amino acids, wherein Xaa at positions 71 and 72 are Phe and Trp;
(xix) SEQ ID NO: 6, wherein Xaa at positions 72 and 113-116 are wild-type amino acids, wherein each Xaa at positions 70 and 71 is Pro, respectively;
(xx) SEQ ID NO: 6, wherein Xaa at positions 72 and 113-116 are wild-type amino acids, wherein Xaa at positions 70 and 71 is Thr and Pro, respectively;
(xxi) SEQ ID NO: 6, wherein Xaa at positions 71 and 113-116 are wild-type amino acids, wherein Xaa at positions 70 and 72 are Thr and Trp, respectively;
(xxii) SEQ ID NO: 6, wherein Xaa at positions 70 and 113-116 are wildtype amino acids, wherein Xaa at positions 71 and 72 are Pro and Trp, respectively;
(xxiii) SEQ ID NO: 6, wherein Xaa at positions 72 and 113-116 are wild-type amino acids, wherein Xaa at positions 70 and 71 are Thr and Phe, respectively;
(xxiv) SEQ ID NO: 6, wherein Xaa at positions 113-116 are wild-type amino acids, wherein Xaa at positions 70 to 72 are Pro, Phe, and Trp, respectively;
(xxv) SEQ ID NO: 6, wherein Xaa at positions 70-72, 113, and 116 are wild-type amino acids, wherein Xaa at positions 114 and 115 are Leu and Tyr, respectively;
(xxvi) SEQ ID NO: 6, wherein Xaa at positions 70-72, 113, 115, and 116 are wild-type amino acids, wherein Xaa at position 114 is Ala;
(xxvii) SEQ ID NO: 6, wherein Xaa at positions 70-72, 113, 115, and 116 are wild-type amino acids, wherein Xaa at position 114 is Leu;
(xxviii) SEQ ID NO: 6, wherein Xaa at positions 70-72, 113, 115, and 116 are wild-type amino acids, wherein Xaa at position 114 is Glu;
(xxix) SEQ ID NO: 6, wherein Xaa at positions 70-72, 113, 114, and 116 are wild-type amino acids, wherein Xaa at position 115 is Ala;
(xxx) SEQ ID NO: 6, wherein Xaa at positions 70-72, 113, 114, and 116 are wild-type amino acids, wherein Xaa at position 115 is Leu;
(xxxi) SEQ ID NO: 6, wherein Xaa at positions 70-72, 113, 114, and 116 are wild-type amino acids, wherein Xaa at position 115 is Tyr;
(xxxii) SEQ ID NO: 6, wherein Xaa at positions 70-72, 113, 114, and 116 are wild-type amino acids, wherein Xaa at position 115 is Glu;
(xxxiii) SEQ ID NO: 6, wherein Xaa at positions 70-72, 113, 114, and 116 are wild-type amino acids, wherein Xaa at position 115 is Lys;
(xxxiv) SEQ ID NO: 6, wherein Xaa at positions 70-72 and 113 are wild-type amino acids, wherein Xaa at positions 114-116 are Leu, Leu, and Asp, respectively;
(xxxv) SEQ ID NO: 6, wherein Xaa at positions 70-72, 113, and 116 are wild-type amino acids, wherein each Xaa at positions 114 and 115 are Leu;
(xxxvi) SEQ ID NO: 6, wherein Xaa at positions 70-72, 113, and 116 are wild-type amino acids, wherein Xaa at positions 114 and 115 are Glu and Tyr, respectively;
(xxxvii) SEQ ID NO: 6, wherein Xaa at positions 70-72, 115, and 116 are wild-type amino acids, wherein Xaa at positions 113 and 114 are Leu and Pro, respectively;
(xxxviii) SEQ ID NO: 11, wherein Xaa at positions 71 to 73 are wild-type amino acids, wherein Xaa at position 70 is Val; and
(xxxix) SEQ ID NO: 11, wherein Xaa at positions 70, 72, and 73 are wild-type amino acids, wherein Xaa at position 71 is Met;
or a combination thereof;
wherein the wild-type amino acids of SEQ ID NO: 5 at positions 70-73 and 115-117 are Gly, Ala, Gly, Ile, Val, Gly, and Asn, respectively,
wherein the wild-type amino acids of SEQ ID NO: 6 at positions 70-72 and 113-116 are Gln, Ser, Ser, Pro, Thr, Ser, and Gly, respectively,
wherein the wild-type amino acids of SEQ ID NO: 11 at positions 70-73 are Thr, Ala, Gly, and Thr, respectively.

The amino acid sequences of Group II are analogous to the amino acid sequences of Group I, in that the same modifications are specified. However, the SEQ ID NOs: referred to in Group II are the full-length amino acid sequences of an immature TCR chain, whereas the SEQ ID NOs: referred to in Group I are the amino acid sequences of the germline encoded variable regions plus a portion of the CDR3 of an immature TCR.

The modified TCRs of the invention can additionally comprise a second chain of a TCR heterodimer, or the variable region thereof. For instance, if the modified TCR comprises an alpha chain with amino acid substitutions, the modified TCR can additionally comprise the wild-type beta chain. In this regard, the modified TCR comprising an amino acid sequence selected from the group consisting of (i) to (ix) (in reference to Group I as set forth above) can also comprise SEQ ID NO: 4, which is the germline encoded variable region and part of the CDR3 of the alpha chain of the immature 1G4 NY-ESO-1-specific TCR. The modified TCR comprising an amino acid sequence selected from the group consisting of (x) to (xxviii) of Group I can also comprise SEQ ID NO: 3, which is the germline encoded variable region and part of the CDR3 of the beta chain of the immature 1G4 TCR. Further, the modified TCR comprising an amino acid sequence selected from the group consisting of (xxviii) and (xxix) of Group I can additionally comprise SEQ ID NO: 14, which is the wild-type germline encoded variable region and part of the CDR3 of the immature alpha chain of the DMF5 (F5) MART-1-specific TCR.

Also, if, for example, the modified TCR comprises a full length alpha chain with amino acid substitutions, the modified TCR can additionally comprise the wild-type full-length beta chain. In this regard, the modified TCR comprising an amino acid sequence selected from the group consisting of (i) to (ix) (in reference to Group II as set forth above) can also comprise SEQ ID NO: 2, which is the wild-type full-length alpha chain of the immature 1G4 NY-ESO-1-specific TCR. The modified TCR comprising an amino acid sequence selected from the group consisting of (x) to (xxviii) of Group II can also comprise SEQ ID NO: 1, which is the wild-type full-length beta chain of the immature 1G4 TCR. Further, the modified TCR comprising an amino acid sequence selected from the group consisting of (xxviii) and (xxix) of Group II can additionally comprise SEQ ID NO: 13, which is the wild-type full-length alpha chain of the immature DMF5 (F5) MART-1-specific TCR.

Alternatively, the modified TCRs can be paired with a second TCR chain which is modified (e.g., comprises one or more amino acid substitutions). Preferably, when the modified TCR comprises the amino acid sequence of (x) or (xxv) of Group I, the modified TCR comprises the amino acid sequence (i) of Group I, SEQ ID NO: 7, wherein Xaa at positions 70, 72, 73, and 115-117 are wild-type amino acids and Xaa at position 71 is Ile, or SEQ ID NO: 7, wherein Xaa at positions 72, 73, and 115-117 are wild-type amino acids and Xaa at positions 70 and 71 are Ala and Ile, respectively.

Also preferred is that, when the modified TCR comprises the amino acid sequence of (x) or (xxv) of Group II, the modified TCR comprises the amino acid sequence (i) of Group II, SEQ ID NO: 5, wherein Xaa at positions 70, 72, 73, and 115-117 are wild-type amino acids and Xaa at position 71 is Ile, or SEQ ID NO: 5, wherein Xaa at positions 72, 73, and 115-117 are wild-type amino acids and Xaa at positions 70 and 71 are Ala and Ile, respectively.

The modified TCRs of the invention can comprise one or more immature TCR chains comprising a leader sequence or one or more mature chains in which the leader sequence has been cleaved off. As one of ordinary skill in the art appreciates, the leader sequence of a TCR chain comprises the amino acids at the N-terminus which together serve as a signal to transport the TCR to the plasma membrane and which amino acids are cleaved off to yield the mature form of the TCR. In this regard, the modified TCR can comprise an amino acid sequence selected from Group III or Group IV. Groups III and IV are the same as Groups I and II, respectively, except that the SEQ ID NOs: referred to in Groups III and IV are mature sequences (lack the leader sequence of the TCR chain), whereas the SEQ ID NOs: in Groups I and H are immature sequences comprising the leader sequence.

Group III consists of:
(i) SEQ ID NO: 127, wherein Xaa at positions 51-53 and 96-98 are wild-type amino acids, wherein Xaa at position 54 is Thr;
(ii) SEQ ID NO: 127, wherein Xaa at positions 51 and 96-98 are wild-type amino acids, wherein Xaa at positions 52-54 are Ile, Gln, and Ile, respectively;
(iii) SEQ ID NO: 127, wherein Xaa at positions 51, 53, and 96-98 are wild-type amino acids, wherein Xaa at positions 52 and 54 are Ile and Thr, respectively;
(iv) SEQ ID NO: 127, wherein Xaa at positions 51, 52, and 96-98 are wild-type amino acids, wherein Xaa at positions 53 and 54 are Gln and Thr, respectively;
(v) SEQ ID NO: 127, wherein Xaa at positions 54 and 96-98 are wild-type amino acids, wherein Xaa at positions 51-53 are Ala, Ile, Gln, respectively;
(vi) SEQ ID NO: 127, wherein Xaa at positions 53 and 96-98 are wild-type amino acids, wherein Xaa at positions 51, 52, and 54 are Ala, Ile, and Thr, respectively;
(vii) SEQ ID NO: 127, wherein Xaa at positions 51 and 96-98 are wild-type amino acids, wherein Xaa at positions 52-54 are Ile, Gln, and Thr, respectively;
(viii) SEQ ID NO: 127, wherein Xaa at positions 96-98 are wild-type amino acids, wherein Xaa at positions 51-54 are Ala, Ile, Gln, and Thr respectively;
(ix) SEQ ID NO: 127, wherein Xaa at positions 51-54, 96, and 97 are wild-type amino acids, wherein Xaa at position 98 is Leu;
(x) SEQ ID NO: 128, wherein Xaa at positions 94-97 are wild-type amino acids, wherein Xaa at positions 51-53 are Thr, Pro, and Trp, respectively;
(xi) SEQ ID NO: 128, wherein Xaa at positions 52, 53, and 94-97 are wild-type amino acids, wherein Xaa at position 51 is Pro;
(xii) SEQ ID NO: 128, wherein Xaa at positions 52, 53, and 94-97 are wild-type amino acids, wherein Xaa at position 51 is Thr;
(xiii) SEQ ID NO: 128, wherein Xaa at positions 51, 53, and 94-97 are wild-type amino acids, wherein Xaa at position 52 is Phe;
(xiv) SEQ ID NO: 128, wherein Xaa at positions 51, 53, and 94-97 are wild-type amino acids, wherein Xaa at position 52 is Pro;
(xv) SEQ ID NO: 128, wherein Xaa at positions 51, 52, and 94-97 are wild-type amino acids, wherein Xaa at position 53 is Trp;
(xvi) SEQ ID NO: 128, wherein Xaa at positions 53 and 94-97 are wild-type amino acids, wherein Xaa at positions 51 and 52 are Pro and Phe, respectively;
(xvii) SEQ ID NO: 128, wherein Xaa at positions 52 and 94-97 are wild-type amino acids, wherein Xaa at positions 51 and 53 are Pro and Trp;
(xviii) SEQ ID NO: 128, wherein Xaa at positions 51 and 94-97 are wild-type amino acids, wherein Xaa at positions 52 and 53 are Phe and Trp;
(xix) SEQ ID NO: 128, wherein Xaa at positions 53 and 94-97 are wild-type amino acids, wherein each Xaa at positions 51 and 52 is Pro, respectively;
(xx) SEQ ID NO: 128, wherein Xaa at positions 53 and 94-97 are wild-type amino acids, wherein Xaa at positions 51 and 52 is Thr and Pro, respectively;
(xxi) SEQ ID NO: 128, wherein Xaa at positions 52 and 94-97 are wild-type amino acids, wherein Xaa at positions 51 and 53 are Thr and Trp, respectively;
(xxii) SEQ ID NO: 128, wherein Xaa at positions 51 and 94-97 are wildtype amino acids, wherein Xaa at positions 52 and 53 are Pro and Trp, respectively;
(xxiii) SEQ ID NO: 128, wherein Xaa at positions 53 and 94-97 are wild-type amino acids, wherein Xaa at positions 51 and 52 are Thr and Phe, respectively;
(xxiv) SEQ ID NO: 128, wherein Xaa at positions 94-97 are wild-type amino acids, wherein Xaa at positions 51-53 are Pro, Phe, and Trp, respectively;
(xxv) SEQ ID NO: 128, wherein Xaa at positions 51-53, 94, and 97 are wild-type amino acids, wherein Xaa at positions 95 and 96 are Leu and Tyr, respectively;
(xxvi) SEQ ID NO: 128, wherein Xaa at positions 51-53, 94, 96, and 97 are wild-type amino acids, wherein Xaa at position 95 is Ala;
(xxvii) SEQ ID NO: 128, wherein Xaa at positions 51-53, 94, 96, and 97 are wild-type amino acids, wherein Xaa at position 95 is Leu;
(xxviii) SEQ ID NO: 128, wherein Xaa at positions 51-53, 94, 96, and 97 are wild-type amino acids, wherein Xaa at position 95 is Glu;
(xxix) SEQ ID NO: 128, wherein Xaa at positions 51-53, 94, 95, and 97 are wild-type amino acids, wherein Xaa at position 96 is Ala;
(xxx) SEQ ID NO: 128, wherein Xaa at positions 51-53, 94, 95, and 97 are wild-type amino acids, wherein Xaa at position 96 is Leu;
(xxxi) SEQ ID NO: 128, wherein Xaa at positions 51-53, 94, 95, and 97 are wild-type amino acids, wherein Xaa at position 96 is Tyr;
(xxxii) SEQ ID NO: 128, wherein Xaa at positions 51-53, 94, 95, and 97 are wild-type amino acids, wherein Xaa at position 96 is Glu;
(xxxiii) SEQ ID NO: 128, wherein Xaa at positions 51-53, 94, 95, and 97 are wild-type amino acids, wherein Xaa at position 96 is Lys;
(xxxiv) SEQ ID NO: 128, wherein Xaa at positions 51-53 and 94 are wild-type amino acids, wherein Xaa at positions 95-97 are Leu, Leu, and Asp, respectively;
(xxxv) SEQ ID NO: 128, wherein Xaa at positions 51-53, 94, and 97 are wild-type amino acids, wherein each Xaa at positions 95 and 96 are Leu;
(xxxvi) SEQ ID NO: 128, wherein Xaa at positions 51-53, 94, and 97 are wild-type amino acids, wherein Xaa at positions 95 and 96 are Glu and Tyr, respectively;
(xxxvii) SEQ ID NO: 128, wherein Xaa at positions 51-53, 94, and 97 are wild-type amino acids, wherein Xaa at positions 95 and 96 are Trp and Val, respectively;
(xxxviii) SEQ ID NO: 128, wherein Xaa at positions 51-53, 96, and 97 are wild-type amino acids, wherein Xaa at positions 94 and 95 are Leu and Pro, respectively;
(xxxix) SEQ ID NO: 132, wherein Xaa at positions 52 to 54 are wild-type amino acids, wherein Xaa at position 51 is Val; and
(xxxx) SEQ ID NO: 132, wherein Xaa at positions 51, 53, and 54 are wild-type amino acids, wherein Xaa at position 52 is Met;
or a combination thereof;
wherein the wild-type amino acids of SEQ ID NO: 127 at positions 51-54 and 96-98 are Gly, Ala, Gly, Ile, Val, Gly, and Asn, respectively,
wherein the wild-type amino acids of SEQ ID NO: 128 at positions 51-53 and 94-97 are Gln, Ser, Ser, Pro, Thr, Ser, and Gly, respectively,
wherein the wild-type amino acids of SEQ ID NO: 132 at positions 51-54 are Thr, Ala, Gly, and Thr, respectively.

In a preferred embodiment of the invention, the modified TCR comprising an amino acid sequence selected from Group IV, which consists of:
(i) SEQ ID NO: 125, wherein Xaa at positions 51-53 and 96-98 are wild-type amino acids, wherein Xaa at position 54 is Thr;
(ii) SEQ ID NO: 125, wherein Xaa at positions 51 and 96-98 are wild-type amino acids, wherein Xaa at positions 52-54 are Ile, Gln, and Ile, respectively;
(iii) SEQ ID NO: 125, wherein Xaa at positions 51, 53, and 96-98 are wild-type amino acids, wherein Xaa at positions 52 and 54 are Ile and Thr, respectively;
(iv) SEQ ID NO: 125, wherein Xaa at positions 51, 52, and 96-98 are wild-type amino acids, wherein Xaa at positions 53 and 54 are Gln and Thr, respectively;
(v) SEQ ID NO: 125, wherein Xaa at positions 54 and 96-98 are wild-type amino acids, wherein Xaa at positions 51-53 are Ala, Ile, Gln, respectively;
(vi) SEQ ID NO: 125, wherein Xaa at positions 53 and 96-98 are wild-type amino acids, wherein Xaa at positions 51, 52, and 54 are Ala, Ile, and Thr, respectively;
(vii) SEQ ID NO: 125, wherein Xaa at positions 51 and 96-98 are wild-type amino acids, wherein Xaa at positions 52-54 are Ile, Gln, and Thr, respectively;
(viii) SEQ ID NO: 125, wherein Xaa at positions 96-98 are wild-type amino acids, wherein Xaa at positions 51-54 are Ala, Ile, Gln, and Thr respectively;
(ix) SEQ ID NO: 125, wherein Xaa at positions 51-54, 96, and 97 are wild-type amino acids, wherein Xaa at position 98 is Leu;
(x) SEQ ID NO: 126, wherein Xaa at positions 94-97 are wild-type amino acids, wherein Xaa at positions 51-53 are Thr, Pro, and Trp, respectively;
(xi) SEQ ID NO: 126, wherein Xaa at positions 52, 53, and 94-97 are wild-type amino acids, wherein Xaa at position 51 is Pro;
(xii) SEQ ID NO: 126, wherein Xaa at positions 52, 53, and 94-97 are wild-type amino acids, wherein Xaa at position 51 is Thr;
(xiii) SEQ ID NO: 126, wherein Xaa at positions 51, 53, and 94-97 are wild-type amino acids, wherein Xaa at position 52 is Phe;
(xiv) SEQ ID NO: 126, wherein Xaa at positions 51, 53, and 94-97 are wild-type amino acids, wherein Xaa at position 52 is Pro;
(xv) SEQ ID NO: 126, wherein Xaa at positions 51, 52, and 94-97 are wild-type amino acids, wherein Xaa at position 53 is Trp;
(xvi) SEQ ID NO: 126, wherein Xaa at positions 53 and 94-97 are wild-type amino acids, wherein Xaa at positions 51 and 52 are Pro and Phe, respectively;
(xvii) SEQ ID NO: 126, wherein Xaa at positions 52 and 94-97 are wild-type amino acids, wherein Xaa at positions 51 and 53 are Pro and Trp;
(xviii) SEQ ID NO: 126, wherein Xaa at positions 51 and 94-97 are wild-type amino acids, wherein Xaa at positions 52 and 53 are Phe and Trp;
(xix) SEQ ID NO: 126, wherein Xaa at positions 53 and 94-97 are wild-type amino acids, wherein each Xaa at positions 51 and 52 is Pro, respectively;
(xx) SEQ ID NO: 126, wherein Xaa at positions 53 and 94-97 are wild-type amino acids, wherein Xaa at positions 51 and 52 is Thr and Pro, respectively;
(xxi) SEQ ID NO: 126, wherein Xaa at positions 52 and 94-97 are wild-type amino acids, wherein Xaa at positions 51 and 53 are Thr and Trp, respectively;
(xxii) SEQ ID NO: 126, wherein Xaa at positions 51 and 94-97 are wildtype amino acids, wherein Xaa at positions 52 and 53 are Pro and Trp, respectively;
(xxiii) SEQ ID NO: 126, wherein Xaa at positions 53 and 94-97 are wild-type amino acids, wherein Xaa at positions 51 and 52 are Thr and Phe, respectively;
(xxiv) SEQ ID NO: 126, wherein Xaa at positions 94-97 are wild-type amino acids, wherein Xaa at positions 51-53 are Pro, Phe, and Trp, respectively;
(xxv) SEQ ID NO: 126, wherein Xaa at positions 51-53, 94, and 97 are wild-type amino acids, wherein Xaa at positions 95 and 96 are Leu and Tyr, respectively;
(xxvi) SEQ ID NO: 126, wherein Xaa at positions 51-53, 94, 96, and 97 are wild-type amino acids, wherein Xaa at position 95 is Ala;
(xxvii) SEQ ID NO: 126, wherein Xaa at positions 51-53, 94, 96, and 97 are wild-type amino acids, wherein Xaa at position 95 is Leu;
(xxviii) SEQ ID NO: 126, wherein Xaa at positions 51-53, 94, 96, and 97 are wild-type amino acids, wherein Xaa at position 95 is Glu;
(xxix) SEQ ID NO: 126, wherein Xaa at positions 51-53, 94, 95, and 97 are wild-type amino acids, wherein Xaa at position 96 is Ala;
(xxx) SEQ ID NO: 126, wherein Xaa at positions 51-53, 94, 95, and 97 are wild-type amino acids, wherein Xaa at position 96 is Leu;
(xxxi) SEQ ID NO: 126, wherein Xaa at positions 51-53, 94, 95, and 97 are wild-type amino acids, wherein Xaa at position 96 is Tyr;
(xxxii) SEQ ID NO: 126, wherein Xaa at positions 51-53, 94, 95, and 97 are wild-type amino acids, wherein Xaa at position 96 is Glu;
(xxxiii) SEQ ID NO: 126, wherein Xaa at positions 51-53, 94, 95, and 97 are wild-type amino acids, wherein Xaa at position 96 is Lys;
(xxxiv) SEQ ID NO: 126, wherein Xaa at positions 51-53 and 94 are wild-type amino acids, wherein Xaa at positions 95-97 are Leu, Leu, and Asp, respectively;
(xxxv) SEQ ID NO: 126, wherein Xaa at positions 51-53, 94, and 97 are wild-type amino acids, wherein each Xaa at positions 95 and 96 are Leu;
(xxxvi) SEQ ID NO: 126, wherein Xaa at positions 51-53, 94, and 97 are wild-type amino acids, wherein Xaa at positions 95 and 96 are Glu and Tyr, respectively;
(xxxvii) SEQ ID NO: 126, wherein Xaa at positions 51-53, 94, and 97 are wild-type amino acids, wherein Xaa at positions 95 and 96 are Trp and Val, respectively;
(xxxviii) SEQ ID NO: 126, wherein Xaa at positions 51-53, 96, and 97 are wild-type amino acids, wherein Xaa at positions 94 and 95 are Leu and Pro, respectively;
(xxxix) SEQ ID NO: 131, wherein Xaa at positions 52 to 54 are wild-type amino acids, wherein Xaa at position 51 is Val; and
(xxxx) SEQ ID NO: 131, wherein Xaa at positions 51, 53, and 54 are wild-type amino acids, wherein Xaa at position 52 is Met;
or a combination thereof;
wherein the wild-type amino acids of SEQ ID NO: 125 at positions 51-54 and 96-98 are Gly, Ala, Gly, Ile, Val, Gly, and Asn, respectively,
wherein the wild-type amino acids of SEQ ID NO: 126 at positions 51-53 and 94-97 are Gln, Ser, Ser, Pro, Thr, Ser, and Gly, respectively,
wherein the wild-type amino acids of SEQ ID NO: 131 at positions 51-54 are Thr, Ala, Gly, and Thr, respectively.

The amino acid sequences of Group IV are analogous to the amino acid sequences of Group III, in that the same modifications are specified. However, the SEQ ID NOs: referred to in Group IV are the full-length amino acid sequences of a mature TCR chain, whereas the SEQ ID NOs: referred to in Group III are the amino acid sequences of the germline encoded variable regions plus a portion of the CDR3 of a mature TCR chain.

The modified TCRs comprising the amino acid sequence of any of Group III or IV can additionally comprise a second chain of a TCR heterodimer, or the variable region thereof. For instance, if the modified TCR comprises an alpha chain with amino acid substitutions, the modified TCR can additionally comprise the wild-type beta chain. In this regard, the modified TCR comprising an amino acid sequence selected from the group consisting of (i) to (ix) (in reference to Group III as set forth above) can also comprise SEQ ID NO: 124, which is the germline encoded variable region and part of the CDR3 of the mature alpha chain of the 1G4 NY-ESO-1-specific TCR. The modified TCR comprising an amino acid sequence selected from the group consisting of (x) to (xxviii) of Group III can also comprise SEQ ID NO: 123, which is the germline encoded variable region and part of the CDR3 of the mature beta chain of the 1G4 TCR. Further, the modified TCR comprising an amino acid sequence selected from the group consisting of (xxviii) and (xxix) of Group III can additionally comprise SEQ ID NO: 14, which is the wild-type germline encoded variable region and part of the CDR3 of the immature alpha chain of the DMF5 (F5) MART-1-specific TCR, or a mature form of SEQ ID NO: 14.

Also, if, for example, the modified TCR comprises a full length alpha chain with amino acid substitutions, the modified TCR can additionally comprise the wild-type full-length beta chain. In this regard, the modified TCR comprising an amino acid sequence selected from the group consisting of (i) to (ix) (in reference to Group IV as set forth above) can also comprise SEQ ID NO: 122, which is the wild-type full-length alpha chain of the mature 1G4 NY-ESO-1-specific TCR. The modified TCR comprising an amino acid sequence selected from the group consisting of (x) to (xxviii) of Group IV can also comprise SEQ ID NO: 121, which is the wild-type full-length beta chain of the mature 1G4 TCR. Further, the modified TCR comprising an amino acid sequence selected from the group consisting of (xxviii) and (xxix) of Group IV can additionally comprise SEQ ID NO: 13, which is the wild-type full-length immature alpha chain of the DMF5 (F5) MART-1-specific TCR, or a mature form thereof.

Alternatively, the modified TCRs can be paired with a second TCR chain which is modified (e.g., comprises one or more amino acid substitutions). Preferably, when the modified TCR comprises the amino acid sequence of (x) or (xxv) of Group III, the modified TCR comprises the amino acid sequence (i) of Group III, SEQ ID NO: 127, wherein Xaa at positions 51, 53, 54, and 96-98 are wild-type amino acids and Xaa at position 52 is Ile, or SEQ ID NO: 127, wherein Xaa at positions 53, 54, and 96-98 are wild-type amino acids and Xaa at positions 51 and 52 are Ala and Ile, respectively.

Also preferred is that, when the modified TCR comprises the amino acid sequence of (x) or (xxv) of Group IV, the modified TCR comprises the amino acid sequence (i) of Group IV, SEQ ID NO: 125, wherein Xaa at positions 51, 53, 54, and 96-98 are wild-type amino acids and Xaa at position 52 is Ile, or SEQ ID NO: 125, wherein Xaa at positions 53, 54, and 96-98 are wild-type amino acids and Xaa at positions 51 and 52 are Ala and Ile, respectively.

Also provided by the invention is an isolated or purified polypeptide comprising a functional portion of any of the modified TCRs described herein, wherein the functional portion comprises the amino acid substitutions. The term “polypeptide” as used herein includes oligopeptides and refers to a single chain of amino acids connected by one or more peptide bonds.

With respect to the inventive polypeptides, the functional portion can be any portion comprising contiguous amino acids of the modified TCR of which it is a part, provided that the functional portion comprises the amino acid substitutions. The term “functional portion” when used in reference to a modified TCR refers to any part or fragment of the modified TCR of the invention, which part or fragment retains the biological activity of the modified TCR of which it is a part (the parent modified TCR). Functional portions encompass, for example, those parts of a modified TCR that retain the ability to recognize target cells, or detect, treat, or prevent a disease, to a similar extent, the same extent, or to a higher extent, as the parent modified TCR. In reference to the parent modified TCR, the functional portion can comprise, for instance, about 10%, 25%, 30%, 50%, 68%, 80%, 90%, 95%, or more, of the parent TCR.

The functional portion can comprise additional amino acids at the amino or carboxy terminus of the portion, or at both termini, which additional amino acids are not found in the amino acid sequence of the parent modified TCR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent modified TCR.

The polypeptide can comprise a functional portion of either or both of the α and β chains of the TCRs of the invention, such as a functional portion comprising one of more of CDR1, CDR2, and CDR3 of the variable region(s) of the α chain and/or β chain of a TCR of the invention. In this regard, the polypeptide can comprise the amino acid sequence of SEQ ID NOs: 7, 8, or 12 with the amino acid substitutions designated herein. The polypeptides can additionally comprise the amino acid sequence of a second TCR chain. For example, the polypeptides can additional comprise the amino acid sequence of SEQ ID NO: 4, if the polypeptide comprises the amino acid sequence of any of (i) to (ix) (in reference Group I), SEQ ID NO: 3, if the polypeptide comprises the amino acid sequence of any of (x) to (xxviii) of Group I, or SEQ ID NO: 14, if the polypeptide comprises the amino acid sequence of any of (xxviii) to (xxix) of Group I. Also, for example, the polypeptides can additionally comprise the amino acid sequence of SEQ ID NO: 124, if the polypeptide comprises the amino acid sequence of any of (i) to (ix) (in reference Group III), SEQ ID NO: 123, if the polypeptide comprises the amino acid sequence of any of (x) to (xxviii) of Group III, or SEQ ID NO: 14, if the polypeptide comprises the amino acid sequence of any of (xxviii) to (xxix) of Group III.

Alternatively or additionally, the inventive polypeptide can comprise the entire length of an α or β chain of one of the modified TCRs described herein. In this regard, the inventive polypeptide can comprise an amino acid sequence selected from the group consisting of SEQ ID NOs: 5, 6, or 11 with the amino acid substitutions designated herein. Alternatively, the polypeptide of the invention can comprise both chains of the modified TCRs as described herein. For example, the inventive polypeptide can comprise both amino acid sequences of SEQ ID NOs: 5 and 2. The inventive polypeptide can comprise both amino acid sequences of SEQ ID NOs: 6 and 1 or can comprise both amino acid sequences of SEQ ID NO: 11 and 13.

The invention further provides an isolated or purified protein comprising at least one of the inventive polypeptides described herein. By “protein” is meant a molecule comprising one or more polypeptide chains.

The protein of the invention can comprise the amino acid sequence of any of the modified TCRs described herein. For instance, the protein can comprise an amino acid sequence selected from Group I or Group III. Alternatively, the protein can comprise an amino acid sequence selected from Group II or IV.

Alternatively, if, for example, the protein comprises a single polypeptide chain comprising the amino acid sequence of (i) of Group I and SEQ ID NO: 4, or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods. See, for instance, Choi et al., Mol. Biotechnol. 31: 193-202 (2005).

The protein of the invention can be a recombinant antibody comprising at least one of the inventive polypeptides described herein. As used herein, “recombinant antibody” refers to a recombinant (e.g., genetically engineered) protein comprising at least one of the polypeptides of the invention and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab)₂ fragment of an antibody, etc. The polypeptide chain of an antibody, or portion thereof, can exist as a separate polypeptide of the recombinant antibody. Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein.

Included in the scope of the invention are functional variants of the inventive modified TCRs, polypeptides, and proteins described herein. The term “functional variant” as used herein refers to a modified TCR, polypeptide, or protein having substantial or significant sequence identity or similarity to a parent modified TCR, polypeptide, or protein, which functional variant retains the biological activity of the modified TCR, polypeptide, or protein of which it is a variant. Functional variants encompass, for example, those variants of the TCR, polypeptide, or protein described herein (the parent modified TCR, polypeptide, or protein) that retain the ability to recognize target cells to a similar extent, the same extent, or to a higher extent, as the parent modified TCR, polypeptide, or protein. In reference to the parent modified TCR, polypeptide, or protein, the functional variant can, for instance, be at least about 30%, 50%, 75%, 80%, 90%, 98% or more identical in amino acid sequence to the parent modified TCR, polypeptide, or protein.

The functional variant can, for example, comprise the amino acid sequence of the parent TCR, polypeptide, or protein with at least one conservative amino acid substitution. Alternatively or additionally, the functional variants can comprise the amino acid sequence of the parent modified TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent modified TCR, polypeptide, or protein.

The modified TCR, polypeptide, or protein can consist essentially of the specified amino acid sequence or sequences described herein, such that other components e.g., other amino acids, do not materially change the biological activity of the functional variant.

The modified TCRs, polypeptides, and proteins of the invention (including functional portions and functional variants) can be of any length, i.e., can comprise any number of amino acids, provided that the modified TCRs, polypeptides, or proteins (or functional portions or functional variants thereof) retain their biological activity, e.g., the ability to specifically bind to antigen, detect diseased cells in a host, or treat or prevent disease in a host, etc. For example, the polypeptide can be 50 to 5000 amino acids long, such as 50, 70, 75, 100, 125, 150, 175, 200, 300, 400, 500, 600, 700, 800, 900, 1000 or more amino acids in length. In this regard, the polypeptides of the invention also include oligopeptides.

The modified TCRs, polypeptides, and proteins of the invention (including functional portions and functional variants) of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The modified TCRs, polypeptides, and proteins of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

When the modified TCRs, polypeptides, and proteins of the invention (including functional portions and functional variants) are in the form of a salt, preferably, the polypeptides are in the form of a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The modified TCR, polypeptide, and/or protein of the invention (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the TCRs, polypeptides, and proteins of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the TCRs, polypeptides, and/or proteins described herein (including functional portions and functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive TCRs, polypeptides, and proteins can be synthetic, recombinant, isolated, and/or purified.

Included in the scope of the invention are conjugates, e.g., bioconjugates, comprising any of the inventive modified TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al., Inorg Chem. 44(15): 5405-5415 (2005)).

Further provided by the invention is a nucleic acid comprising a nucleotide sequence encoding any of the modified TCRs, polypeptides, or proteins described herein (including functional portions and functional variants thereof).

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl)uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any nucleotide sequence which encodes any of the modified TCRs, polypeptides, or proteins, or functional portions or functional variants thereof. Alternatively, the nucleotide sequence can comprise a nucleotide sequence which is degenerate to any of the sequences or a combination of degenerate sequences.

The invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ, plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter operably linked to the nucleotide sequence encoding the modified TCR, polypeptide, or protein (including functional portions and functional variants thereof), or to the nucleotide sequence which is complementary to or which hybridizes to the nucleotide sequence encoding the modified TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

The invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant modified TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL). More preferably, the host cell is a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), peripheral blood mononuclear cells (PBMCs), peripheral blood leukocytes (PBLs), tumor infiltrating cells (TILs), memory T cells, naïve T cells, and the like. Preferably, the T cell is a CD8⁺ T cell or a CD4⁺ T cell.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The invention further provides an antibody, or antigen binding portion thereof, which specifically binds to an epitope of the modified TCR of the invention, wherein the epitope comprises the one or more amino acid substitutions. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of the inventive modified TCR. Desirably, the antibody is specific for the epitope of the inventive modified TCR comprising the amino acid substitutions, such that there is minimal cross-reaction with other peptides or proteins.

Methods of testing antibodies for the ability to bind to any functional portion of the inventive modified TCR are known in the art and include any antibody-antigen binding assay, such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

Suitable methods of making antibodies are known in the art. For instance, standard hybridoma methods are described in, e.g., Köhler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5^{th} Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display furthermore can be used to generate the antibody of the invention. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3^{rd} Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

The invention also provides antigen binding portions of any of the antibodies described herein. The antigen binding portion can be any portion that has at least one antigen binding site, such as Fab, F(ab′)₂, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)). Antibody fragments of the invention, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The inventive modified TCRs, polypeptides, proteins, (including functional portions and functional variants thereof), nucleic acids, recombinant expression vectors, host cells (including populations thereof), and antibodies (including antigen binding portions thereof), can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, or can be 100%.

The inventive modified TCRs, polypeptides, proteins (including functional portions and variants thereof), nucleic acids, recombinant expression vectors, host cells (including populations thereof), and antibodies (including antigen binding portions thereof), all of which are collectively referred to as “inventive TCR materials” hereinafter, can be formulated into a composition, such as a pharmaceutical composition. In this regard, the invention provides a pharmaceutical composition comprising any of the modified TCRs, polypeptides, proteins, functional portions, functional variants, nucleic acids, expression vectors, host cells (including populations thereof), and antibodies (including antigen binding portions thereof), and a pharmaceutically acceptable carrier. The inventive pharmaceutical compositions containing any of the inventive TCR materials can comprise more than one inventive TCR material, e.g., a polypeptide and a nucleic acid, or two or more different modified TCRs. Alternatively, the pharmaceutical composition can comprise an inventive TCR material in combination with another pharmaceutically active agents or drugs, such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

With respect to pharmaceutical compositions, the pharmaceutically acceptable carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. The following formulations for oral, aerosol, parenteral, subcutaneous, intravenous, intramuscular, intraarterial, intrathecal, interperitoneal, rectal, and vaginal administration are exemplary and are in no way limiting. More than one route can be used to administer the inventive TCR materials, and in certain instances, a particular route can provide a more immediate and more effective response than another route.

Formulations suitable for oral administration can consist of (a) liquid solutions, such as an effective amount of the inventive TCR material dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the inventive TCR material in a flavor, usually sucrose and acacia or tragacanth, as well as pastilles comprising the inventive TCR material in an inert base, such as gelatin and glycerin, or sucrose and acacia, emulsions, gels, and the like containing, in addition to, such excipients as are known in the art.

The inventive TCR material, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inventive TCR material can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the inventive TCR material in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)). Preferably, when administering cells, e.g., dendritic cells, the cells are administered via injection.

Additionally, the inventive TCR materials, or compositions comprising such inventive TCR materials, can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the inventive TCR materials of the invention can be formulated as inclusion complexes, such as cyclodextrin inclusion complexes, or liposomes.

For purposes of the invention, the amount or dose of the inventive TCR material administered should be sufficient to effect, e.g., a therapeutic or prophylactic response, in the subject or animal over a reasonable time frame. For example, the dose of the inventive TCR material should be sufficient to bind to antigen, or detect, treat or prevent disease in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive modified TCR, polypeptide, or protein upon administration of a given dose of such T cells to a mammal among a set of mammals of which is each given a different dose of the T cells, could be used to determine a starting dose to be administered to a mammal. The extent to which target cells are lysed or IFN-γ is secreted upon administration of a certain dose can be assayed by methods known in the art, including, for instance, the methods described herein as Example 3.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the condition being treated. By way of example and not intending to limit the invention, the dose of the inventive TCR material can be about 0.001 to about 1000 mg/kg body weight of the subject being treated/day, from about 0.01 to about 10 mg/kg body weight/day, about 0.01 mg to about 1 mg/kg body weight/day.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a linker to a targeting moiety. The practice of conjugating compounds, e.g., inventive TCR materials, to targeting moieties is known in the art. See, for instance, Wadwa et al., J. Drug Targeting 3: 111 (1995) and U.S. Pat. No. 5,087,616. The term “targeting moiety” as used herein, refers to any molecule or agent that specifically recognizes and binds to a cell-surface receptor, such that the targeting moiety directs the delivery of the inventive TCR materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). The term “linker” as used herein, refers to any agent or molecule that bridges the inventive TCR materials to the targeting moiety. One of ordinary skill in the art recognizes that sites on the inventive TCR materials, which are not necessary for the function of the inventive TCR materials, are ideal sites for attaching a linker and/or a targeting moiety, provided that the linker and/or targeting moiety, once attached to the inventive TCR materials, do(es) not interfere with the function of the inventive TCR materials, i.e., the ability to bind to antigen, or to detect, treat, or prevent disease.

Alternatively, the inventive TCR materials can be modified into a depot form, such that the manner in which the inventive TCR materials is released into the body to which it is administered is controlled with respect to time and location within the body (see, for example, U.S. Pat. No. 4,450,150). Depot forms of inventive TCR materials can be, for example, an implantable composition comprising the inventive TCR materials and a porous or non-porous material, such as a polymer, wherein the inventive TCR materials is encapsulated by or diffused throughout the material and/or degradation of the non-porous material. The depot is then implanted into the desired location within the body and the inventive TCR materials are released from the implant at a predetermined rate.

It is contemplated that the inventive pharmaceutical compositions, modified TCRs, polypeptides, proteins, nucleic acids, recombinant expression vectors, host cells, or populations of cells can be used in methods of treating or preventing a disease in a host. Without being bound to a particular theory, the inventive modified TCRs are believed to have enhanced biological activity, e.g., ability to recognize antigen, such that the modified TCR (or related inventive polypeptide or protein) when expressed by a cell is able to mediate a stronger immune response against the cell expressing the antigen for which the modified TCR is specific. In this regard, the invention provides a method of treating or preventing a disease in a host, comprising administering to the host any of the pharmaceutical compositions in an amount effective to treat or prevent the disease in the host.

The disease can be any disease involving an antigen, e.g., an infectious disease, an autoimmune disease, a cancer.

For purposes herein, “infectious disease” means a disease that can be transmitted from person to person or from organism to organism, and is caused by a microbial agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, and influenza.

For purposes herein, “autoimmune disease” refers to a disease in which the body produces an immunogenic (i.e., immune system) response to some constituent of its own tissue. In other words the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. Preferably, the cancer is melanoma.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Also provided is a method of detecting a diseased cell in a host, wherein the diseased cell expresses an antigen characteristic of a disease. The method comprises (i) contacting a sample comprising cells of the host with any of the inventive modified TCRs, polypeptides, proteins, nucleic acids, recombinant expression vectors, host cells, and populations of host cells described herein, thereby forming a complex between the antigen which is characteristic of the disease and the inventive modified TCR, polypeptide, protein, nucleic acid, recombinant expression vector, host cell, or population of cells, and (ii) detecting the complex, wherein detection of the complex is indicative of a diseased cell in the host.

The diseased cell can be any cell of any disease, which cell expresses an antigen that is characteristic of the disease. The diseased cell can be a cancer cell or an infected cell, for example. Preferably, the diseased cell is a melanoma cell

In the method of treating or preventing a disease or of detecting a diseased cell, the inventive modified TCR has antigenic specificity for an antigen that is characteristic of the disease to be treated, prevented, or detected. For instance, if the disease to be treated, prevented or detected is melanoma, the inventive modified TCR has antigenic specificity for a melanoma antigen, e.g., MART-1, NY-ESO-1, gp100, etc. If a host cell or a population comprising at least one host cell is used in the method, the host cell desirably expresses a TCR having antigenic specificity for the antigen of the disease. If an inventive nucleic acid or recombinant expression vector is used in the method, the nucleic acid or recombinant expression vector desirably encodes the modified TCR which has antigenic specificity for an antigen of the disease to be treated, prevented, or detected, such that expression of the nucleic acid or recombinant expression vector is achieved in a cell and the TCR expressed by the cell is capable of binding to the antigen of the disease.

With respect to the inventive method of detecting a diseased cell in a host, the sample comprising cells of the host can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the host, e.g., the cells of any organ or tissue, including blood cells.

For purposes of the inventive detecting method, the contacting step can take place in vitro or in vivo with respect to the host. Preferably, the contacting is an in vitro step.

Also, detection of the complex can occur through any number of ways known in the art. For instance, the inventive modified TCRs, polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered to the host, the cells can be cells that are allogeneic or autologous to the host. Preferably, the cells are autologous to the host.

The host referred to herein can be any host. Preferably, the host is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Methods of expressing nucleic acids in cells (including CD4+ T cells and CD8+ T cells) are known in the art, as discussed herein. Preferably, the nucleic acid is an RNA and the RNA is expressed in T cells by methods described in Example 1.

The T cells can be assayed for the ability to recognize target cells and for antigen specificity employing methods known in the art. Preferably, the T cells are assayed as described herein as Example 1 or 3.

### EXAMPLES

The following examples further illustrate the invention but, of course, should not be construed as in any way limiting its scope.

The following examples describe the amino acid substitutions of the modified TCRs with respect to the mature TCR sequence (i.e., the TCR sequence without the leader sequence).

### Example 1

This example demonstrates the construction of inventive modified TCRs and the biological activity thereof.

The regions upstream and downstream of sequence encoding the amino acid substitutions (AAS) are amplified in two separate PCRs using WT 1G4 a or 13 constructs as templates and also are designed to contain overlapping regions that allow them to be linked in a third PCR. The sequences of the forward and reverse primers that are used to generate the amino terminus of the α chains (SEQ ID NOs: 43 and 44), as well as the sequences of the primers that are used to generate similar β chain fragments (SEQ ID NOs: 66 and 67) are listed in Table 2.

TABLE 2

| | Region | | | |
|---|---|---|---|---|
| | and | Primer | | |
| TCR | Chain | Name | Sequence (5′ to 3′) | SEQ ID NO: |
| 1G4 | CDR3α | α1.1F | ATATTAATACGACTCACTATAGGGCACCATGGAGACCCTGCTGGGC | 43 |
| 1G4 | CDR3α | α2.1R | CCGCACAGCGCACAGGTAGG | 44 |
| 1G4 | CDR3α | α3.1F | CCTACCTGTGCGCTGTGCGGGCCACCAGCGGCGGCAGCTAC | 45 |
| 1G4 | CDR3α | α3.2F | CCTACCTGTGCGCTGTGCGGCTGACCAGCGGCGGCAGCTAC | 46 |
| 1G4 | CDR3α | α3.3F | CCTACCTGTGCGCTGTGCGGCCTGCCAGCGGCGGCAGCTAC | 47 |
| 1G4 | CDR3α | α3.4F | CCTACCTGTGCGCTGTGCGGCCTCTGAGCGGCGGCAGCTAC | 48 |
| 1G4 | CDR3α | α3.5F | CCTACCTGTGCGCTGTGCGGCCTTACAGCGGCGGCAGCTAC | 49 |
| 1G4 | CDR3α | α3.6F | CCTACCTGTGCGCTGTGCGGCCTGAGAGCGGCGGCAGCTAC | 50 |
| 1G4 | CDR3α | α3.7F | CCTACCTGTGCGCTGTGCGGCCTAAGAGCGGCGGCAGCTAC | 51 |
| 1G4 | CDR3α | α3.8F | CCTACCTGTGCGCTGTGCGGCCTCCCAGCGGCGGCAGCTAC | 52 |
| 1G4 | CDR3α | α3.9F | CCTACCTGTGCGCTGTGCGGCCTACCGCCGGCGGCAGCTAC | 53 |
| 1G4 | CDR3α | α3.10F | CCTACCTGTGCGCTGTGCGGCCTACCCTGGGCGGCAGCTAC | 54 |
| 1G4 | CDR3α | α3.11F | CCTACCTGTGCGCTGTGCGGCCTACCTACGGCGGCAGCTAC | 55 |
| 1G4 | CDR3α | α3.12F | CCTACCTGTGCGCTGTGCGGCCTACCGAGGGCGGCAGCTAC | 56 |
| 1G4 | CDR3α | α3.13F | CCTACCTGTGCGCTGTGCGGCCTACCAAGGGCGGCAGCTAC | 57 |
| 1G4 | CDR3α | α3.14F | CCTACCTGTGCGCTGTGCGGCCTACCCCCGGCGGCAGCTAC | 58 |
| 1G4 | CDR3α | α3.15F | CCTACCTGTGCGCTGTGCGGCCTACCAGCGCCGGCAGCTAC | 59 |
| 1G4 | CDR3α | α3.16F | CCTACCTGTGCGCTGTGCGGCCTACCAGCGAGGGCAGCTAC | 60 |
| 1G4 | CDR3α | α3.17F | CCTACCTGTGCGCTGTGCGGCCTACCAGCAAGGGCAGCTAC | 61 |
| 1G4 | CDR3α | α3.18F | CCTACCTGTGCGCTGTGCGGCCTACCAGCCCCGGCAGCTAC | 62 |
| 1G4 | CDR3α | α3.19F | CCTACCTGTGCGCTGTGCGGCCCCTGCTGGGCGGCAGCTACATCC | 63 |
| 1G4 | CDR3α | α3.20F | CCTACCTGTGCGCTGTGCGGCCCCTGTACGGCGGCAGCTACATCC | 64 |
| 1G4 | CDR3α | α4.1R | T₍₆₆₎AGCTGCTCCACAGCCGCAG | 65 |
| 1G4 | CDR2β | β1.1F | ATATTAATACGACTCACTATAGGGATGAGCATCGGCCTGCTGTG | 66 |
| 1G4 | CDR2β | β2.1R | CACAGAGTAGTGGATCAGCCG | 67 |
| 1G4 | CDR2β | β3.1F | CGGCTGATCCACTACTCTGTGGCCGCCGGAATCACCGACCAGGGCGAG | 68 |
| 1G4 | CDR2β | β3.2F | CGGCTGATCCACTACTCTGTGGGAATCGGAATCACCGACCAGGGCGAG | 69 |
| 1G4 | CDR2β | β3.3F | CGGCTGATCCACTACTCTGTGGGAGCCCAGATCACCGACCAGGGCGAG | 70 |
| 1G4 | CDR2β | β3.4F | CGGCTGATCCACTACTCTGTGGGAGCCGGAACCACCGACCAGGGCGAG | 71 |
| 1G4 | CDR2β | β3.5F | CGGCTGATCCACTACTCTGTGGCCATCGGAATCACCGACCAGGGCGAG | 72 |
| 1G4 | CDR2β | β3.6F | CGGCTGATCCACTACTCTGTGGCCGCCGGAACCACCGACCAGGGCGAG | 73 |
| 1G4 | CDR2β | β3.7F | CGGCTGATCCACTACTCTGTGGGAATCGGAACCACCGACCAGGGCGAG | 74 |
| 1G4 | CDR2β | β3.8F | CGGCTGATCCACTACTCTGTGGCCATCGGAACCACCGACCAGGGCGAG | 75 |
| 1G4 | CDR2β | β4.1R | T₍₆₆₎AGCCCCGGCTGTCCTTCC | 76 |
| 1G4 | CDR3β | β1.1F | ATATTAATACGACTCACTATAGGGATGAGCATCGGCCTGCTGTG | 77 |
| 1G4 | CDR3β | β2.1R | ATAGCTGCTGGCGCAGAAGTAC | 78 |
| 1G4 | CDR3β | β3.9F | GTACTTCTGCGCCAGCAGCTATGCCGGCAACACCGGCGAGC | 79 |
| 1G4 | CDR3β | β3.10F | GTACTTCTGCGCCAGCAGCTATCTGGGCAACACCGGCGAGC | 80 |
| 1G4 | CDR3β | β3.11F | GTACTTCTGCGCCAGCAGCTATTACGGCAACACCGGCGAGC | 81 |
| 1G4 | CDR3β | β3.12F | GTACTTCTGCGCCAGCAGCTATGAGGGCAACACCGGCGAGC | 82 |
| 1G4 | CDR3β | β3.13F | GTACTTCTGCGCCAGCAGCTATGTGGCCAACACCGGCGAGC | 83 |
| 1G4 | CDR3β | β3.14F | GTACTTCTGCGCCAGCAGCTATGTGGGCGCCACCGGCGAGC | 84 |
| 1G4 | CDR3β | β3.15F | GTACTTCTGCGCCAGCAGCTATGTGGGCGACACCGGCGAGC | 85 |
| 1G4 | CDR3β | β3.16F | GTACTTCTGCGCCAGCAGCTATGTGGGCCTGACCGGCGAGC | 86 |
| 1G4 | CDR3β | β3.17F | GTACTTCTGCGCCAGCAGCTATGTGGGCAAGACCGGCGAGC | 87 |
| 1G4 | CDR3β | β3.18F | GTACTTCTGCGCCAGCAGCTATGTGGGCCCCACCGGCGAGC | 88 |
| 1G4 | CDR3β | β4.1R | T₍₆₆₎AGCCCCGGCTGTCCTTCC | 89 |

Primers are according to a codon optimized 1G4 sequence; codons encoding AAS are shown in bolded and underlined text.

The carboxy terminal TCR fragments are generated using a second set of forward primers (SEQ ID NOs: 45-64, 68-75, and 79-88), containing nucleotide sequences that are complementary to the α2R or β2R primers, followed by sequence encoding AAS and 9 to 26 additional nucleotides at the 3′ end corresponding to downstream sequence. The α3 and β3 primers (SEQ ID NOs: 45-64, 68-75, and 79-88), which vary depending upon the desired sequence alterations, are used to carry out a second PCR with the corresponding α4 or β4 reverse primers (SEQ ID NOs: 76 and 89), which consist of 64-66 d(T) residues at the 5′ end followed by sequence complementary to the 3′ end of the α or β constant region coding sequences. The fragments encoding the amino and carboxy termini of the α or β TCRs are purified on 2% E-Gels (Invitrogen, Carlsbad, Calif.), and then combined for use as templates for a third PCR, which is carried out with the α1F and α4R (SEQ ID NOs: 43 and 65) or β1F and β4R (SEQ ID NOs: 66 and 76) primers and generates full length α and β constructs encoding AAS in either the CDR2 or CDR3 regions.

Amplifications are carried out with 1 unit/50 μl of Phusion high-fidelity DNA polymerase (New England Biolabs, Ipswich, Mass.), 0.5 μM primers, and 0.5 μM dNTP by incubation at 98° C. for 30 seconds, followed by 35 cycles of amplification at 98° C. for 20 seconds, 58° C. for 20 seconds, and 72° C. for 20 seconds. The PCR products are sequenced as previously described (Robbins et al, J. Immunol. 173: 7125-7130 (2004)), and are used as template for in vitro mRNA transcription reactions. Codon optimized versions of the 1G4 α and β chains are used to generate TCR variants that are used to carry out the initial screening assays. The forward primer used to amplify the 1G4 constructs contain a T7 RNA polymerase binding site. Transcripts of the 1G4 anti-NY-ESO-1 TCR are generated from PCR products using the mMESSAGE mMACHINE T7 High Yield Capped RNA Transcription kit (Ambion, Austin, Tex.).

DMF4 and DMF5 anti-MART-1 TCR variants are constructed in like manner to the 1G4 variants, except that the primers in Table 3 are used. Also, the forward primers used to amplify the DMF4 and DMF5 variants contain T3 RNA polymerase binding sites, and transcripts are generated from PCR products using the mMES SAGE mMACHINE T3 High Yield Capped RNA Transcription kit (Ambion).

TABLE 3

| | Region | | | SEQ ID |
|---|---|---|---|---|
| TCR | and Chain | Primer Name | Sequence (5′ to 3′) | NO: |
| DMF4 | CDR2β | β1.4F | GACTAATTAACCCTCACTAAAGGGACACCATGGGCACAAGGTTGTTCTTC | 90 |
| DMF4 | CDR2β | β2.4R | GTAATGGATCAGCCTCAGCC | 91 |
| DMF4 | CDR2β | β3.20F | GCTGAGGCTGATCCATTACTCATATGCCGTTAAAGATACTGACAAAGGAGAAGTC | 92 |
| DMF4 | CDR2β | β3.21F | GCTGAGGCTGATCCATTACTCATATGCCGCCAAAGATACTGACAAAGGAGAAGTC | 93 |
| DMF4 | CDR2β | β3.22F | GCTGAGGCTGATCCATTACTCATATGCCGTTGCCGATACTGACAAAGGAGAAGTC | 94 |
| DMF4 | CDR2β | β3.23F | GCTGAGGCTGATCCATTACTCATATGCCGTTAAAGCCACTGACAAAGGAGAAGTC | 95 |
| DMF4 | CDR2β | β4.4R | T₍₆₅₎CAGAAATCCTTTCTCTTGACCATGGC | 96 |
| DMF5 | CDR2β | β1.5F | GACTAATTAACCCTCACTAAAGGGACACCATGAGAATCAGGCTCCTGTGCT | 97 |
| DMF5 | CDR2β | β2.5R | TGAATAATGGATGAGCCTTAGC | 98 |
| DMF5 | CDR2β | β3.24F | GCTAAGGCTCATCCATTATTCAAATGCCGCAGGTACCACTGGCAAAGGAGAAGTCC | 99 |
| DMF5 | CDR2β | β3.25F | GCTAAGGCTCATCCATTATTCAAATATCGCAGGTACCACTGGCAAAGGAGAAGTCC | 100 |
| DMF5 | CDR2β | β3.26F | GCTAAGGCTCATCCATTATTCAAATACTATCGGTACCACTGGCAAAGGAGAAGTCC | 101 |
| DMF5 | CDR2β | β3.27F | GCTAAGGCTCATCCATTATTCAAATACTGTGGGTACCACTGGCAAAGGAGAAGTCC | 102 |
| DMF5 | CDR2β | β3.28F | GCTAAGGCTCATCCATTATTCAAATACTGCAGCCACCACTGGCAAAGGAGAAGTCC | 103 |
| DMF5 | CDR2β | β3.29F | GCTAAGGCTCATCCATTATTCAAATACTGCAGGTGCCACTGGCAAAGGAGAAGTCC | 104 |
| DMF5 | CDR2β | β3.30F | GCTAAGGCTCATCCATTATTCAAATACTGCAGGTATCACTGGCAAAGGAGAAGTCC | 105 |
| DMF5 | CDR2β | β4.3R | T₍₆₅₎CAGAAATCCTTTCTCTTGACCATGGC | 106 |

Codons encoding AAS are shown in bolded and underlined text.

Transient T cell transfections are carried out as previously described (Zhao et al., Mol. Ther. 13: 151-159 (2006)). Briefly, PBMC are initially stimulated using 30 ng/ml of the soluble anti-CD3 OKT3 for 5 to 21 days and subsequently electroporated with in vitro transcribed RNA at a concentration of 2 μG per 10⁶ T cells Two to four hours later, cytokine stimulation assays are initiated.

For experiments carried out using CD8+ T cells, cells are separated using anti-CD8 magnetic beads (Miltenyi Biotec, Auburn, Calif.). For experiments carried out with CD4+ and CD8+ T cells, the CD4+ T cells are initially separated using anti-CD4 magnetic beads (Miltenyi Biotec) using MS or LS positive selections columns (Miltenyi Biotec), resulting in cells that were >95% CD4 single positive. The cells that passed through the CD4 selection column are run through a LD column (Miltenyi Biotec), incubated with anti-CD8 magnetic beads, and selected on an MS or LS column. The isolated CD8+ T cells are >95% CD8 single positive. Transduced T cells are assayed four to 14 days later for cytokine release in co-culture assays.

Expression of cell surface markers are carried out using FITC- or PE-conjugated antibodies directed against TCRBV13.1, which are obtained from Immunotech (Westbrook, Me.), CD3, CD4, and CD8 or from BD Biosciences (San Jose, Calif.). The relative log fluorescence of live cells is measured using either a FACScan or a FACScanto flow cytometer (BD Biosciences). Tetramers are produced by the NIH Tetramer Core Facility (Atlanta, Ga.).

Cytokine release is measured following the incubation of 5×10⁴ to 10⁵ T cells with 10⁵ target cells in 200 μl for 18 hours, as previously described (Robbins et al., Cancer Res. 54: 3124-3126 (1994)).

Evaluation of AAS in the Alpha Chain of the NY-ESO-1-Specific 1G4 TCR

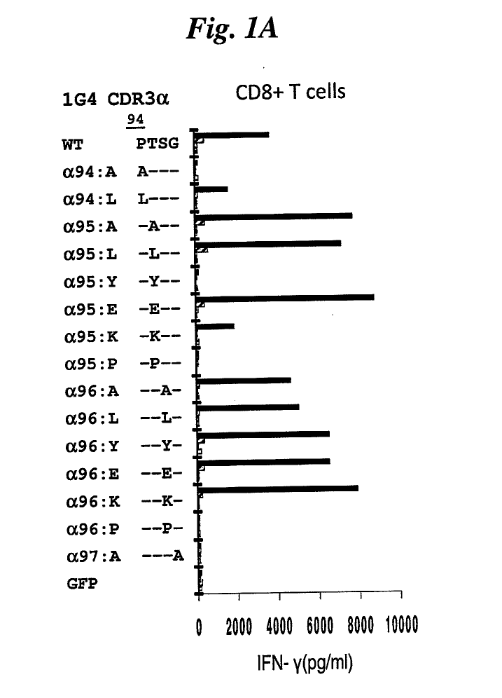

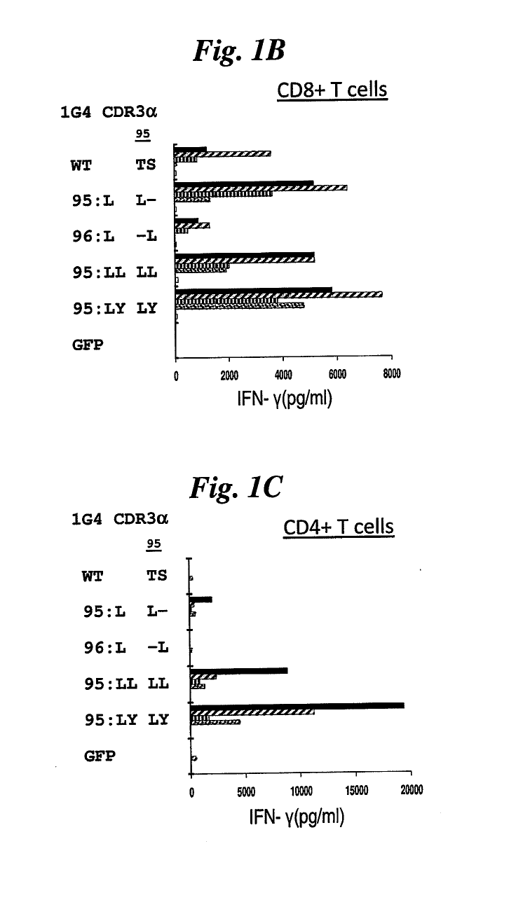

Amino acids 94 to 97 of the 1G4 α chain (PTSG; SEQ ID NO: 26), which correspond to the amino terminus of the CDR3α loop (Chen et al., J. Exp. Med. 201: 1243-1255 (2005)), are initially targeted for alteration. Transfection of whole PBMC with TCR variants containing substitution of alanine or leucine for proline at position 94 (designated 1G4α94:A and 1G4α94:L, respectively) results in diminished recognition of peptide pulsed T2 cells (FIG. 1A). Conversely, substitution of alanine, leucine, or glutamic acid for the threonine residue at position 95 appears to lead to enhanced recognition of target cells pulsed with 1 μM of the NY-ESO-1:157-165 peptide. A variety of the substitutions, some of which appear to increase and others that appear to decrease reactivity of CD8+ T cells, are shown in FIG. 1A.

Preliminary screening assays fail to result in the identification of individual AAS within the 1G4 CDR2a region that enhance T cell recognition. Accordingly, further studies are not carried out.

The ability of tumor cells that naturally process and present the NY-ESO-1:157-165 epitope to stimulate CD8+ and CD4+ T cells that express modified 1G4 TCRs containing single or dual AAS is examined next. Transfection of CD8+ T cells with the WT 1G4β chain, as well as the α95:L, the α95:LL, or the α95:LY constructs enhance the response of transfected CD8+ T cells to antigen positive tumor targets. Conversely, the response of CD8+ T cells transfected with the α96:L appears to be diminished in comparison with cells transfected with the WT α and β TCR (FIG. 1B). The CD4+ T cells transfected with the WT 1G4 TCR fail to release significant levels of IFN-γ in response to antigen⁺/HLA-A2⁺ tumors (FIG. 1C). CD4+ T cells transfected with the α95:L or α96:L constructs also fail to recognize the antigen⁺/HLA-A2⁺ tumors. In contrast, CD4+ T cells transfected with the α95:LL and α95:LY constructs secrete levels of IFN-γ in response to the antigen⁺/HLA-A2⁺ tumor targets that are comparable to or higher than those generated by CD8+ T cells transfected with the same constructs (FIG. 1C). Responses directed against NY-ESO-1⁻ tumor targets are not observed in CD8+ or CD4+ T cells transfected with the WT or variant TCRs, indicating that the TCR modifications do not cause non-specific target cell recognition by the transfected T cells.

Evaluation of AAS in the Beta Chain of 1G4

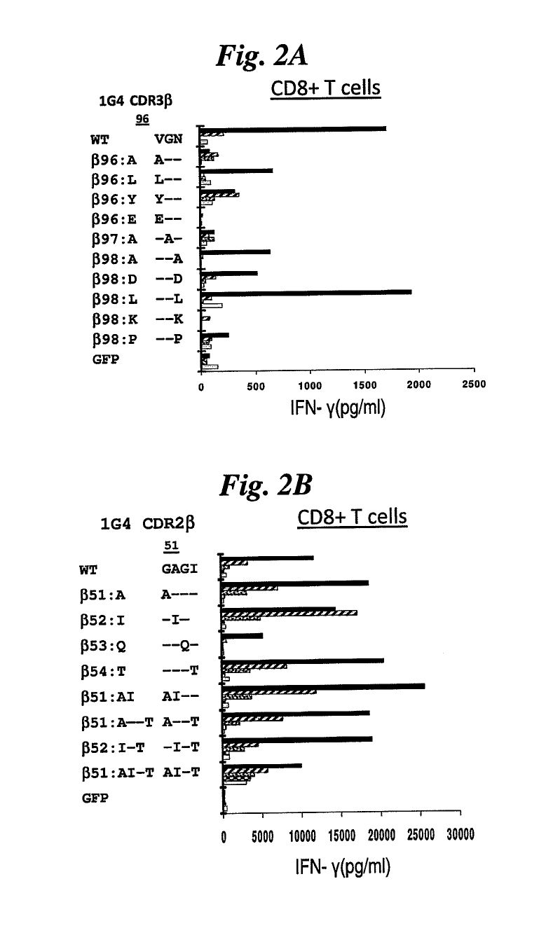

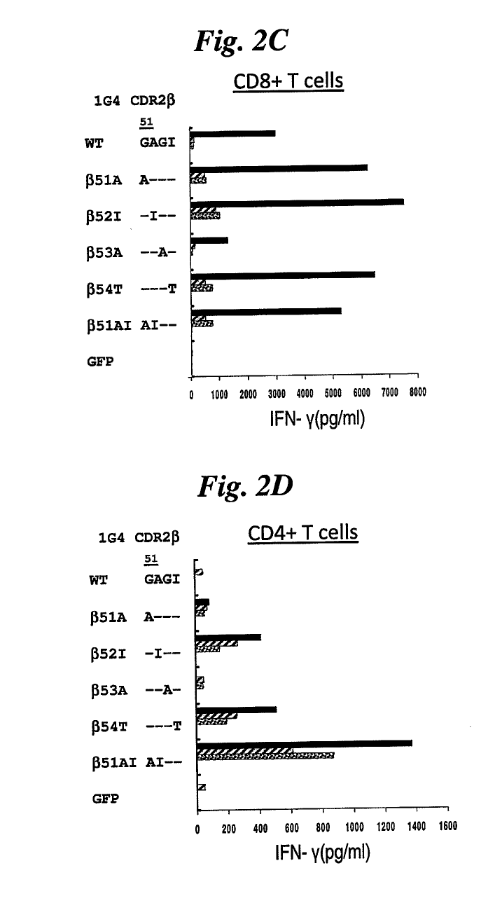

The responses of T cells transfected with 1G4 variants containing amino acid substitutions of CDR3 residues 96-98 in the 1G4 β chain (which correspond to the sequence VGN) of WT) are evaluated. While some substitutions lead to diminished recognition of peptide pulsed target cells (FIG. 2A), none of the substitutions in the CDR3 of the β region significantly enhance the function of transfected T cells.

The effects of modifications of CDR2β residues on T cell function are examined next. The results of a preliminary test indicate that CD8+ T cells transfected with the WT 1G4 α construct and constructs encoding conservative substitutions of the beta chain (β51:A, β52:I and β54:T) lead to enhanced recognition of peptide pulsed target cells. The non-conservative substitution β53:Q, in contrast, leads to diminished peptide recognition (FIG. 2B). Transfection of constructs encoding dual AAS at these positions (β51:AI, β51:A→T and β52:I→T) with the WTα construct do not further enhance the activity of transfected CD8+ T cells when compared to cells transfected with corresponding single AAS. Transfection of a β chain construct encoding three AAS at positions 51, 52 and 54 (β51:AI-T) along with the WTα construct, however, lead to the non-specific recognition of T2 cells that are pulsed with an irrelevant peptide (FIG. 2B).

In accordance with the results obtained using peptide pulsed targets described immediately above, transfection of CD8+ T cells with either of the three 1G4 TCR variants β51:A, β52:I or β54:T in conjunction with the WT 1G4 α chain construct results in enhanced recognition of the HLA-A2⁺/NY-ESO-1⁺ melanoma cell lines (FIG. 2C). Transfection of CD4+ T cells with the WT 1G4 TCR does not lead to significant recognition of the NY-ESO-1⁺/HLA-A2⁺ tumor cell lines, whereas CD4+ T cells transfected with either the β52:I or β54:T constructs along with the 1G4 WTα construct release significant levels of IFN-γ in response to the appropriate tumor targets (FIG. 2D). Transfection of a construct encoding AAS at positions 51 and 52 (β51:AI) appears to further enhance the cytokine levels released in response to tumor cell stimulation by CD4+ T cells, as compared to cells transfected with corresponding single AAS. These dual AAS constructs do not appear to similarly enhance the function of transfected CD8+ T cells (FIGS. 2C and D).

Evaluation of AAS in the β Chain of the F5 TCR

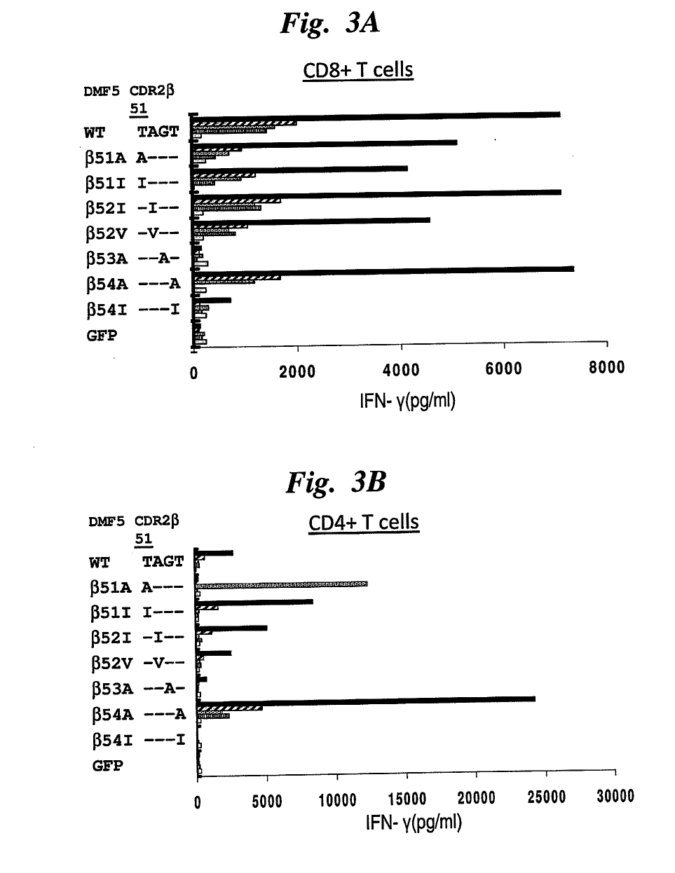

The DMF5 TCR is one of the most potent TCRs among a panel of 24 MART-1 TCRs that are compared in terms of their ability to mediate antigen recognition by gene modified CD8+ T cells and CD4+ T cells (Johnson et al., J. Immunol. 177: 6548-6559 (2006)). Variants of the DMF5 TCR are generated to contain conservative AAS at the CDR2β residues 51 to 54, which correspond to TAGT (SEQ ID NO: 40) in the WT TCR. Populations of CD4+ T cells that are transfected with the WT DMF5 TCR recognize two of the four MART-1⁺/HLA-A2⁺ tumor cell lines tested, although these responses are substantially lower than those observed in CD8+ T cells transfected with the WT TCR (FIGS. 3A and B). Transfection of CD4+ T cells with the 1354:A variant significantly enhances the levels of IFN-γ released in response to the four tumor targets. Transfection of CD8⁺ cells with this variant does not significantly alter T cell response. The substitution of alanine for the glycine residue at position 53, as well as isoleucine for the threonine residue at position 54, reduces the responses of gene modified CD8+ T cells and CD4+ T cells. A conservative AAS of isoleucine for alanine at position 52 does not alter the response of either CD8+ T cells or CD4+ T cells (FIGS. 3A and B). A substitution of alanine for the threonine residue at position 51 does not alter the response of CD8+ T cells, but does significantly enhance the response to one of the antigen⁺/HLA-A2⁺ tumor targets (1359-A2).

A subsequent assay is carried out with a wide variety of target cells that are either HLA-A2^{+/-} and MART-1^{+/-}. This assay provided further evidence that the recognition mediated by the DMF5 β54:A variant was HLA-A2- and MART-1-specific. The β51:A AAS may have generated an allo-reactive TCR, since CD4+ T cells and CD8+ T cells expressing this variant are also stimulated by the HLA-AT parental 1359 tumor cell line (Tables 4 and 5). Substitutions of DMF5 CDR2α residues 50-54, as well as the CDR3α residues 93 and 94, also are generated. None of the variants tested, however, demonstrate an enhanced function of gene modified T cells. These results indicate that the DMF5β chain residue 54 represents a critical residue that can be altered to enhance the function of this TCR in CD4+ T cells.

TABLE 4

| | | | DMF5 CDR2β variant | | | |
|---|---|---|---|---|---|---|
| Target Cells + Peptide | | | TAGT (WT) | AAGT | TAGA | GFP |
| T2 + 1 μM gp100: 154-162 | | | <30 | <30 | <30 | <30 |
| T2 + 1 μM MART-1 | | | 4740 | 7770 | 5180 | <30 |
| T2 + 0.1 μM MART-1 | | | 1420 | 680 | 1320 | <30 |
| T2 + 0.01 μM MART-1 | | | 53 | <30 | 56 | <30 |
| T2 + 1 nM MART-1 | | | <30 | <30 | <30 | <30 |
| Melanoma cells | HLA-A2 | MART-1 | | | | |
| 397-A2 | + | + | 2030 | 1340 | 2160 | 27 |
| 526 | + | + | 1083 | 680 | 671 | <30 |
| 624.38 | + | + | 3660 | 2650 | 3470 | 21 |
| 888-A2 | + | + | 9570 | 7360 | 7020 | 45 |
| 1300 | + | + | 6170 | 4180 | 5380 | 55 |
| 1359-A2 | + | + | 175 | 157 | 207 | 57 |
| 1363 | + | + | 496 | 209 | 595 | <30 |
| SK23 | + | + | 4090 | 2830 | 3790 | 14 |
| 397-A24 | − | + | <30 | <30 | <30 | <30 |
| 624.28 | − | + | <30 | <30 | <30 | <30 |
| 888 | − | + | <30 | <30 | <30 | <30 |
| 1359 | − | + | <30 | 270 | <30 | <30 |
| A375 | + | − | <30 | 68 | 32 | 30 |
| T Alone | | | <30 | <30 | <30 | <30 |

TABLE 5

| | | | DMF5 CDR2β variant | | | |
|---|---|---|---|---|---|---|
| | | | TAGT | | | |
| Target Cells + Peptide | | | (WT) | AAGT | TAGA | GFP |
| T2 + 1 μM gp100: 154-162 | | | <30 | <30 | <30 | <30 |
| T2 + 1 μM MART-1 | | | 1180 | <30 | 4580 | <30 |
| T2 + 0.1 μM MART-1 | | | 5 | <30 | 399 | <30 |
| T2 + 0.01 μM MART-1 | | | <30 | <30 | <30 | <30 |
| T2 + 1 nM MART-1 | | | <30 | <30 | <30 | <30 |
| Tumor cells | HLA-A2 | MART-1 | | | | |
| 397-A2 | + | + | 206 | <30 | 1028 | <30 |
| 526 | + | + | 35 | <30 | 351 | <30 |
| 624.38 | + | + | 250 | <30 | 1287 | <30 |
| 888-A2 | + | + | 2799 | 650 | 4285 | <30 |
| 1300 | + | + | 827 | <30 | 2370 | <30 |
| 1359-A2 | + | + | <30 | 862 | 32 | <30 |
| 1363 | + | + | <30 | <30 | <30 | <30 |
| SK23 | + | + | 187 | <30 | 732 | <30 |
| 397-A24 | − | + | <30 | <30 | <30 | <30 |
| 624.28 | − | + | <30 | <30 | <30 | <30 |
| 888 | − | + | <30 | <30 | <30 | <30 |
| 1359 | − | + | <30 | 3690 | <30 | <30 |
| A375 | + | − | 53 | 20 | 55 | 64 |
| T Alone | na | na | <30 | <30 | <30 | <30 |

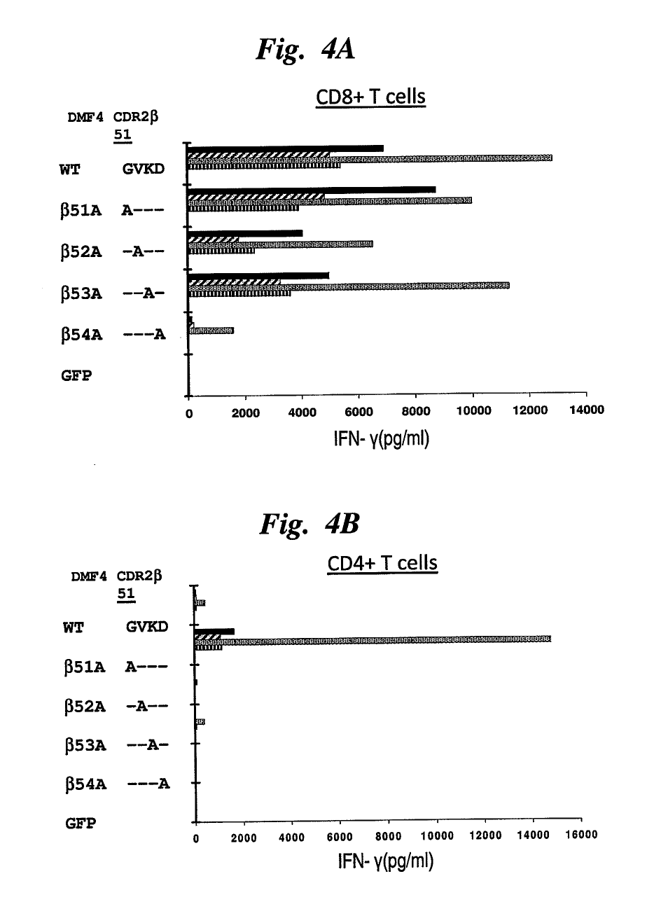

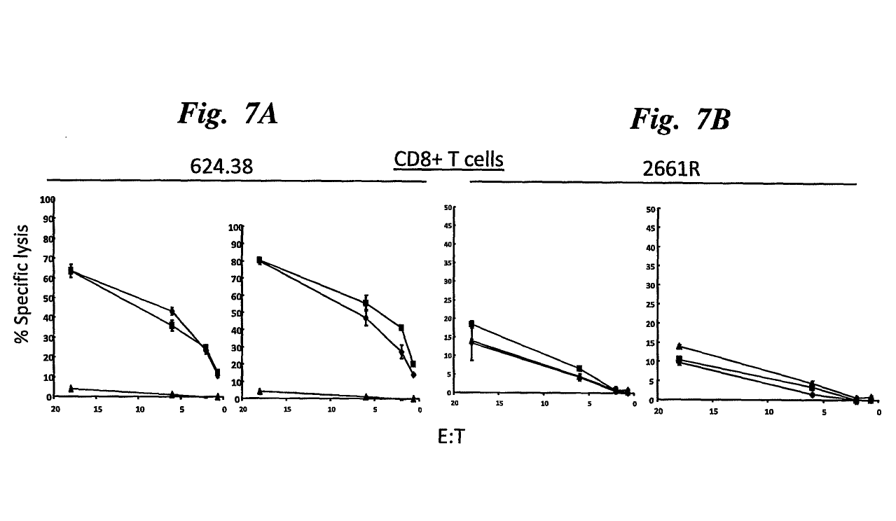

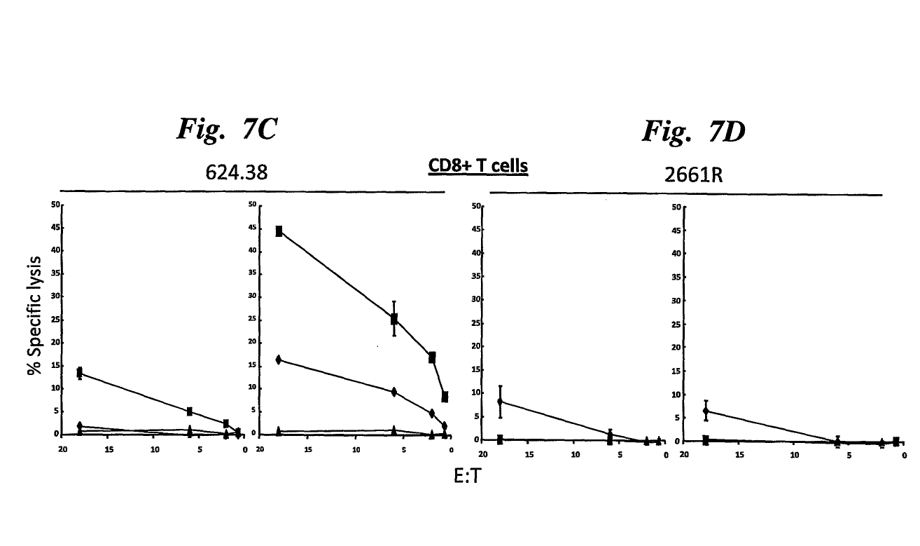

DMF4 is isolated from a dominant TIL clonotype associated with a clinical response to adoptive immunotherapy (Dudley et al., Science 298: 850-854 (2002)). Alanine substitutions of the DMF4 TCR are generated within the CDR2β chain residues 51 to 54, which corresponded to GVKD in the DMF4 WT β chain. Responses directed against HLA-A2⁺/MART-1⁺ positive tumor cells are not significantly altered by transfection of CD8+ T cells with the WT DMF4 α chain along with variants containing substitutions of alanine at either positions 51, 52 or 53 of the DMF4 β chain (FIG. 4A). In contrast, these responses are dramatically reduced in cells transfected with a DMF4 β chain construct containing a substitution of alanine for the aspartic acid residue at position 54. Significant levels of IFN-γ are seen in CD4+ T cells that are transfected with the DMF4 β51:A, but not with the additional DMF4 variants tested. Only a low level of IFN-γ is seen in response to one of the targets, 1300 mel, when CD4+ T cells are transfected with the DMF4 WT TCR (FIG. 4B). The results indicate that these responses are antigen specific, since CD8+ T cells and CD4+ T cells that are transfected with the β51:A construct fail to respond to any of the antigen⁻ or HLA-A2⁻ target cells that are tested (FIG. 7).

This example demonstrated the generation and testing of modified TCRs of the invention.

### Example 2

This example demonstrates that T cells expressing 1G4 TCR variants exhibit enhanced IL-2 secretion.

The ability of transduced T cells to produce IL-2 is evaluated in parallel with IFN-γ responses to antigen stimulation. Co-cultures of T cells and target cells are incubated with 5 μg/ml of the anti-Tac anti-CD25 antibody, which is kindly provided by Dr. Thomas Waldmann, National Institutes of Health, Bethesda, Md.

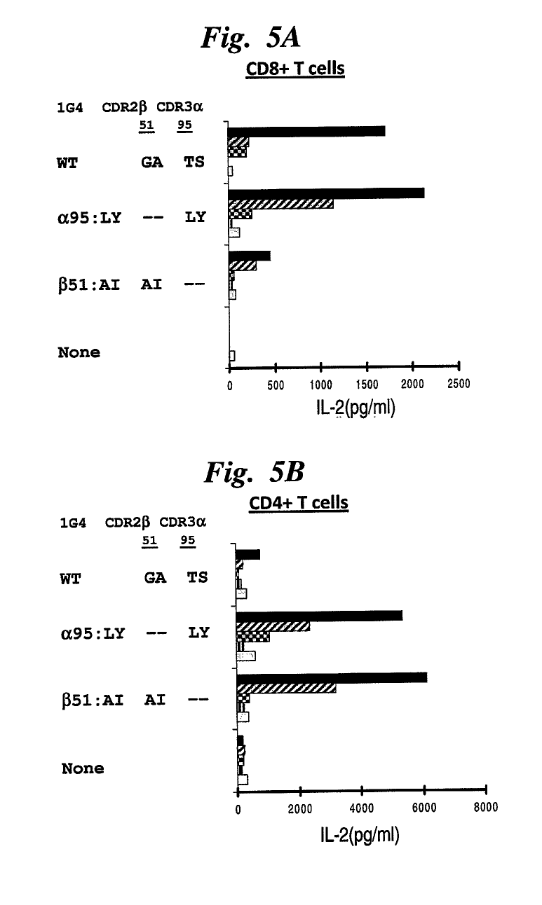

The results demonstrate that CD8+ T cells transduced with the 1G4α95:LY/WT β variant secreted levels of IL-2 in response to the three antigen-positive and HLA-A2-positive tumor cells 624.38 and 1300 that are similar to those secreted by cells transduced with the 1G4 WT TCR, and secreted higher levels of IL-2 in response to the HLA-A2 positive tumor cell line H1299A2, whereas the IL-2 responses of cells transduced with the β51:AI/WT α TCR to the 624.38 and 1300 tumor cells appeared to be lower than those generated with the WT α and β TCR (FIG. 5A). The CD4+ T cells that were transduced with the α95:LY and β51:AI constructs secreted high levels of IL-2 in response to the 624.38, H1299A2 and 1300 tumor cell lines that were higher than those released from cells transduced with the 1G4 WT a and b constructs (FIG. 5B).

This example demonstrated that the modified TCRs of the invention exhibit enhanced IL-2 secretion as compared to WT.

### Example 3

This example demonstrates that NY-ESO-1 TCR variants and MART-1 TCR variants exhibit enhanced lysis of tumor targets.

The effects of TCR modifications on the lytic function of transfected CD8+ and CD4+ T cells are evaluated. The ability of transduced PBL to lyse target cells is measured in ⁵¹Cr release assays, as previously described (Topalian et al., J. Immunol. 142: 3714-3725 (1989)).

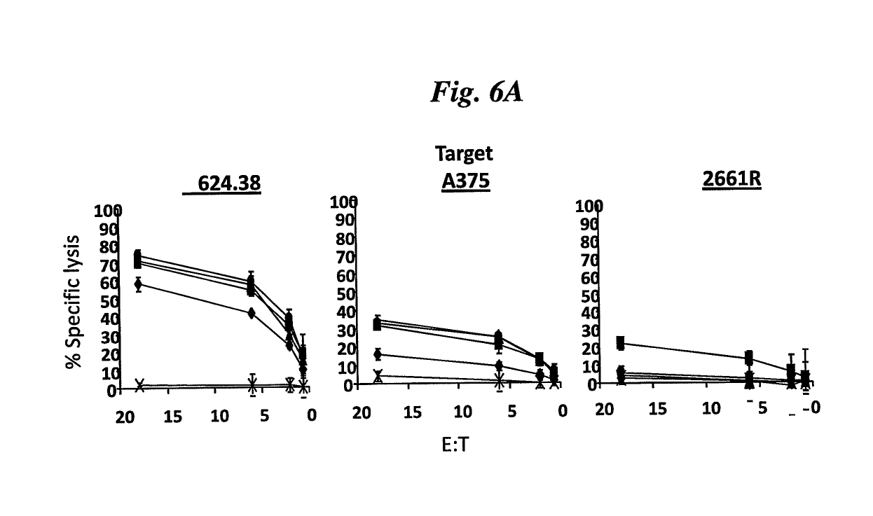

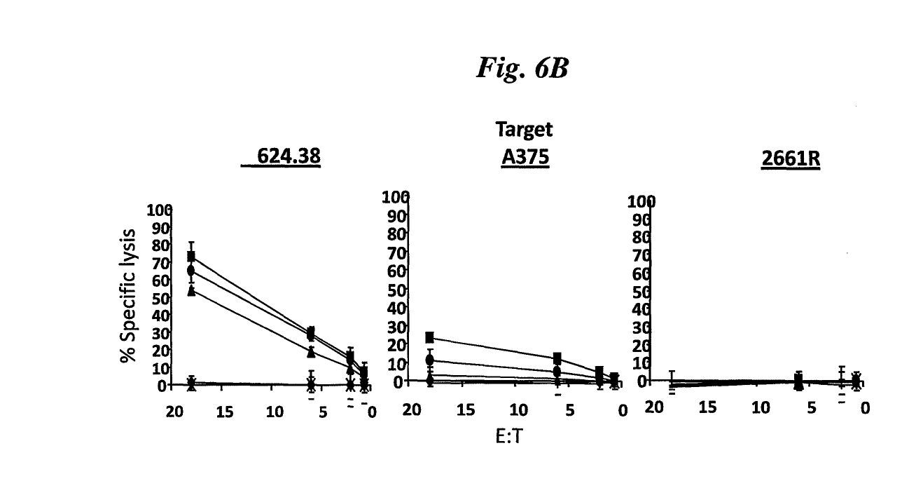

Transfection of the 1G4β51:AI, α95:LL and α95:LY TCR in conjunction with the appropriate WT 1G4 TCRs appear to modestly enhance lysis of two antigen and HLA-A2 positive targets cells, 624.38 and A375, by CD8+ T cells (FIG. 6A). Low levels of lysis of the HLA-A2 positive, but antigen⁻ renal carcinoma cell line (2661R) by CD8+ T cells transfected with the 1G4β51:AI variant, but not the α95:LL or α95:LY constructs, are also observed. Antigen positive target cells are not lysed by CD4+ T cells transfected with the WT 1G4 TCR, whereas CD4+ T cells transfected with the β51:AI, α95:LL and α95:LY constructs demonstrate significant lysis of the 624.38 tumor targets (FIG. 6B). A low level of lysis of the A375 tumor target is also observed with CD4+ T cells transfected with the β51:AI and α95:LY constructs, but the antigen negative 2661R target cell line is not lysed by any of the CD4+ transfectants, but is lysed by CD8+ T cells transfected with the β51:AI construct (FIGS. 6A and B).

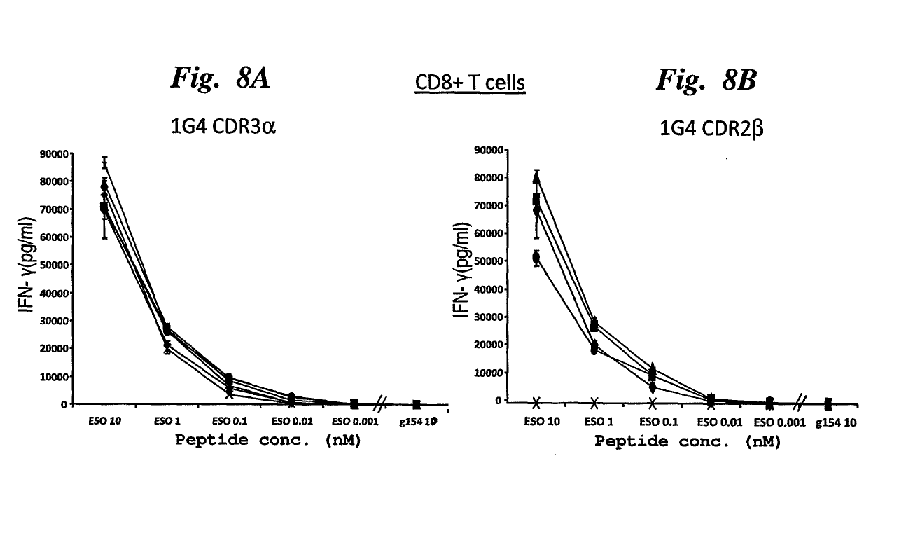

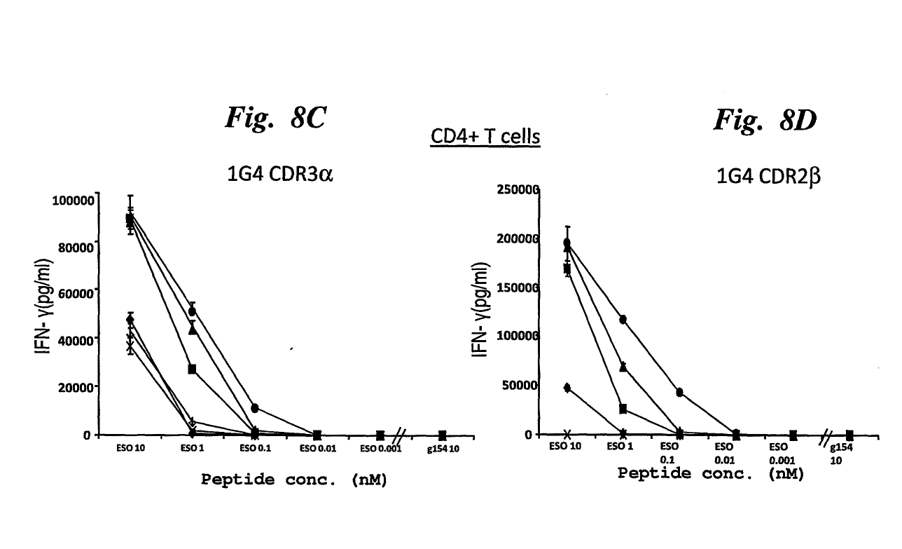

The lytic activities of CD8+ as well as CD4+ T cells that express the DMF4 and DMF5 variants are compared with cells that express the corresponding WT TCRs. The lytic activities of CD8+ T cells expressing either the DMF4 β51:A or the DMF5 β54:A variants directed against the antigen and HLA-A2 positive tumor cell line 624.38 are not significantly higher than those seen with the DMF4 and DMF5 WT TCRs (FIG. 7A). While CD4+ T cells transfected with the DMF4 and DMF5 WT TCRs mediate little or no lysis of 624.38 tumor cells, transfection of the DMF4 β51:A and DMF5 β54:A TCRs along with the corresponding WT TCR α chains enhance the lysis of 624.38 cells mediated by CD4+ T cells (FIG. 7C), which is in agreement with cytokine assay results. At the same time, expression of the modified TCRs in CD8+ or CD4+ T cells does not significantly enhance the lysis of the HLA-A2⁺/antigen⁻ target cell line 2661R (FIGS. 7B and D). Peptide titration studies demonstrate that the DMF4 β51:A construct has a minimal effect on the recognition of peptide pulsed target cells by transfected CD8+ T cells, whereas CD8+ T cells transfected with the DMF5 β54:A variant recognize targets pulsed with between 10 and 100 fold lower concentrations of MART-1 peptide than those transfected with the WT DMF5 TCR (FIG. 8A). Transfected CD4+ T cells expressing either the DMF4 β51:A or DMF5 β4:A variants with the corresponding WT α chains recognized between 10 and 100 fold lower concentrations of the MART-1:27-35 peptide than CD4+ cells transfected with the corresponding WT TCR (FIG. 8B).

This example demonstrated the enhanced lytic activity of the modified TCRs of the invention.

### Example 4

This example demonstrates the stable retroviral transduction of T cells with modified TCRs and the biological activity thereof.

Retroviral constructs are generated from the 1G4 α95:LY and β51:AI TCR variants that are found to enhance T cell function in the assays of the previous examples. These constructs are generated from non-codon optimized transcripts that encode the 1G4 WT α and β amino acid sequences. Initially, non-codon optimized 1G4 WT α and β constructs are generated by replacing the junctional regions present within individual TRAV-21 and TRBV6-5 cDNA clones isolated from polyclonal TIL populations (Robbins et al., J. Immunology 173: 7125-7130 (2004)) with sequences corresponding to the junctional regions of the WT 1G4 α and β chains. The 5′ end of the α chain is generated by amplification of the TRAV-21 cDNA clone using a forward primer corresponding to the 5′ end of the coding region, 5′-CACCATGGAGACCCTCTTGGGC-3′ (SEQ ID NO: 107) and a reverse primer encoding a portion of the WT 1G4 Jα region and the 3′ end of the WT TRAV-21 coding region 5′-CTGGTTCCTCTTCCAAATGTAGGTATGTAGCTTCCTCCTGAT GTGGGCCTCACAGCACAGAGGTAGG-3′ (SEQ ID NO: 108). A second PCR is carried out using a forward primer corresponding to the 1G4 Jα region and the 5′ end of the Cα chain sequence, 5′-CTACATACCTACATTTGGAAGAGGAACCAGCCTTAT TGTTCATC CGTATATC CAGAACCCTGACCC-3′ (SEQ ID NO: 109) and a reverse primer corresponding to the 3′ end of the Ca region, 5′-TCAGCTGGACCACAGCCGCAGC-3′ (SEQ ID NO: 110). The 5′ end of the β chain is generated by amplification of the TRBV-6-5 cDNA clone using a forward primer corresponding to the 5′ end of the coding region, 5′-CACCATGAGCATCGGCCTCCTGTG-3′ (SEQ ID NO: 111) and a reverse primer encoding a portion of the WT 1G4 Dβ and Jβ regions and the 3′ end of the TRBV6-5 coding region 5′-CTCCCCGGTGTTCCCGACGTAACTGCTGGCACAGAAGTAC-3′ (SEQ ID NO: 112). A second PCR is carried out using a forward primer 5′-CAGCAGTTACGTCGGGAA CACCGGGGAGCTGTTT TTTGGAGAAG-3′ (SEQ ID NO: 113) corresponding to the 1G4 Dβ and Jβ regions along with the 5′ end of the Cβ chain sequence and a reverse primer, 5′-CTAGCCTCTGGAATCCTTTCTCTTG-3′ (SEQ ID NO: 114), corresponding to the 3′ end of the Cβ2 region. The resulting native 1G4 α and β products are cloned in the pCR4Blunt-TOPO vector using the Zero Blunt TOPO PCR Cloning Kit (Invitrogen).

In order to generate the 1G4 retroviral TCR variant, the 1G4 α chain is amplified using a primer containing an NcoI site followed by the 5′ coding region of AV21, 5′-TACCATGGAGACCCTCTTGGGCCTGCTTATCCTTTG-3′ (SEQ ID NO: 115), and a reverse primer comprising a short spacer encoding the residues SGSG (SEQ ID NO: 93), followed by sequences encoding the “self-cleaving” P2A sequence (Szymczak et al., Nat. Biotechnol. 22: 589-594 (2004)), and a furin cleavage site for the removal of additional sequences at the carboxy terminus of the TCR α chain (Thomas, Nat. Rev. Mol. Cell. Biol. 3: 753-766 (2002)), 5′-CGCCGGCCTGCTTCAGCAGGCTGAA GTTGGTG GCTCCGGATCCGGACCGCTTGGCCCGGCTGGACCACAGCCGCAG-3′ (SEQ ID NO: 116). The primer used to amplify the WT TCR encodes residues GSG and the P2A sequence, 5′-CTGCGGCTGTGGTCCAGCGGATCCGGAGCCACCAACTTCAGCCTGCTGAAG CAGGCCGGCG (SEQ ID NO: 117). The 1G4 β chain TCR variant is amplified using a forward primer containing the P2A sequence followed by the start site of the BV6-5 V region, 5′-CCTGCTGAAGCAGGCCGGCGACGTGGAGGAGAACCCCGGCCCC ATGAGCATCGGC CTCCTGTG-3′ (SEQ ID NO: 118), and a reverse primer comprising the 3′ end of the Cβ2 constant region, 5′-TTGAATTCTAGCCTCTGGAATCCTTTCTCTTGACCATAGCCATC-3′ (SEQ ID NO: 119). The two PCR products are then purified on 2% E-gels (Invitrogen) and combined in a third PCR containing the α chain forward primer and the P chain reverse primer, which contains an EcoRI site at the 5′ end. Following digestion with NcoI and EcoRI, the PCR product is gel purified and ligated to the MSGV-1 retroviral vector (Hughes et al., Hum. Gene Ther. 16: 457-472 (2005)) that had been digested with NcoI and EcoRI.

Retroviral supernatants are generated by transient transfection of the human embryonic kidney cancer cell line Gβ2-293 (Clontech, Mountain View. CA), which stably expresses the MoMLV gag and pol genes, with plasmids encoding the RD114 feline endogenous virus retroviral envelope and the recombinant retroviral plasmid encoding the TCR α and β chains using Lipofectamine 2000 (Invitrogen) according to the manufacturers' instructions. Supernatants are collected two days and three days following transfection of 293T cells, and transduced into T cells that are activated for three and four days by OKT3 in the presence of 100 IU/ml of recombinant human IL-2.

Additional experiments are carried out using PBMC that are retrovirally transduced as previously described (Hughes et al., 2005, supra). Retrovirally transduced T cells are assayed four to 14 days following transduction for their response in cytokine stimulation assays.

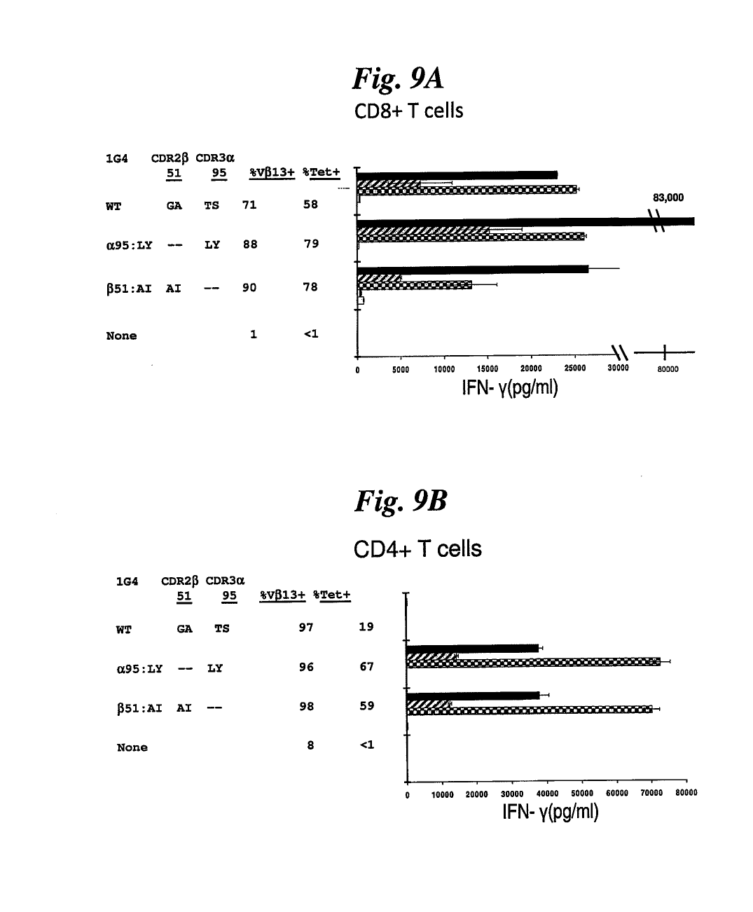

Recombinant retroviruses co-expressing either the WT α and β 1G4 TCR, the α95:LY and WT β TCR, or the WTα and β51:AI TCR are used to stably transduce populations of CD8+ and CD4+ T cells isolated from patient PBMC. The levels of TCR expression in CD8+ T cell transduced with the WT TCR in CD8+ T cells, as determined by staining with an anti-Vβ13 antibody reactive with the 1G4 β chain, appear to be somewhat lower than the levels obtained with the α95:LY and β51:AI constructs, whereas nearly identical levels of Vβ13 expression are observed in CD4+ T cells transduced with each of the three constructs (FIGS. 9A and B). Overall, these results indicate that high levels of transduction are obtained in these cells. Further analysis indicates that similar levels of NY-ESO-1 tetramer binding are observed in CD8+ T cells transduced with the WT, α95:LY or β51:AI constructs, whereas the levels of tetramer staining are significantly higher in CD4+ T cells that are transduced with the α95:LY or β51:AI constructs than in cells transduced with the WT 1G4 TCR (FIGS. 9A and B). The CD8+ T cells that are transduced with retroviruses encoding the 1G4 WTα and β chains, α95:LY and WT β, or WTα and β51:AI constructs generate high levels of IFN-γ when stimulated with the NY-ESO-1 positive, HLA-A2 positive melanoma cell lines 624.38 and 1300, as well as the NY-ESO-1 positive small cell lung carcinoma cell line, H1299-A2 (FIG. 9A). In addition, CD4+ T cells transduced with the α94:LY and β51:AI constructs generate high levels of IFN-γ in response to the three HLA-A2+/NY-ESO-1+ tumor targets (FIG. 9B). These levels are comparable to those observed in CD8+ T cells transduced with the WT or modified TCRs, as previously observed in CD4+ and CD8+ T cells that are transfected with the WT or modified TCRs.

Low levels of cross-reactivity against antigen negative target cells are observed in CD8+ T cells that are transduced with the β51:AI construct, as they release 731 pg/ml of IFN-γ in response to the NY-ESO-1 negative 2661R renal cancer cell line, whereas control un-transduced CD8+ T cells, as well as cells transfected with the WT and α94:LY constructs release background levels of less than 30 pg/ml of IFN-γ in response to the 2661R cell line. The responses of cells transfected with the β51:AI variant directed against 2661R cells are low relative to those directed against the three antigen positive tumor cell lines that are tested; nevertheless, these results are consistent with the low level of 2661 R lysis mediated by CD8+ T cells that were transfected with the (351:AI construct (FIG. 6A).

This example demonstrated that the modified TCRs of the invention have enhanced activity when expressed in CD4⁺ T cells and CD8⁺ T cells via stable retroviral transduction.

### Example 5

This is another example demonstrating the stable retroviral transduction of T cells with modified TCRs and the biological activity thereof.

Random mutations were introduced into the 1G4 anti-NY-ESO-1 TCR α chain at positions 95 and 96 (SEQ ID NO: 126) and the DMF4 anti-MART-1 TCR α chain at positions 93 and 94 (SEQ ID NO: 133) using standard molecular biological methods. Further methods were carried out as essentially described in Example 4. Briefly, a library of retroviral constructs containing the random mutations were generated and pools of 20 constructs were screened by transfection into the GP2-293 packaging cell line along with a plasmid encoding the RD114 retroviral envelope. Transient retroviral supernatants were used to transduce OKT3 stimulated PBMC, which were subsequently separated into CD4+ and CD8+ T cells. The separated cells were then tested for their ability to recognize the NY-ESO-1 and MART-1 positive HLA-A2 positive melanoma cell lines 624.38 and 1300 and the NY-ESO-1 and MART-1 negative HLA-A2 positive renal cancer cell line 2661R.

The results shown in Table 6 demonstrate that the 1G4 variant containing a substitution of WV for the wild type TS had an activity in CD4+ as well as CD8+ T cells comparable to the αLY variant. In addition, the DMF4 variant containing a substitution of LA for the wild type TG sequence had an activity in both CD4+ and CD8+ T cells comparable to the variant containing a substitution of A for the G at position 51 in the wild type DMF4 β chain. Low activity was observed in T cells transduced with the control truncated nerve growth factor (NGFR) construct, as well as for T cells alone.

TABLE 6

| | | | | | | |
|---|---|---|---|---|---|---|
| | | 1G4 | DMF4 | DMF4 | DMF4 | |
| 1G4 TS (WT) | 1G4 αLY | αWV | TG (WT) | αLA | β51A | NGFR |

CD4+ T cells IFN-γ (pg/ml)

| | | | | | | | |
|---|---|---|---|---|---|---|---|
| 624.38 | 29 | 27040 | 37800 | 3220 | 20420 | 15760 | <30 |
| 1300 | 7 | 9660 | 12020 | 3480 | 11060 | 11220 | <30 |
| 2661R | <30 | <30 | <30 | <30 | <30 | <30 | <30 |

CD8+ T cells IFN-γ (pg/ml)

| | | | | | | | |
|---|---|---|---|---|---|---|---|
| T alone | 44 | <30 | 42 | 91 | 205 | 134 | <30 |
| 624.38 | 45750 | 40550 | 46800 | 47700 | 36400 | 32850 | 261 |
| 1300 | 104450 | 75550 | 75550 | 32750 | 91800 | 77950 | 465 |
| 2661R | 56 | 61 | 137 | 523 | 938 | 1092 | <30 |
| T alone | 87 | 83 | 51 | 151 | 135 | 190 | <30 |

### Example 6

This example demonstrates the enhanced biological activity of modified TCRs of the invention.

Experiments are essentially carried out as described in previous Examples. Briefly, three days after OKT-3 stimulation, a pheresis sample is separated using anti-CD8 beads. Ten days after OKT-3 stimulation, the CD8+ T cells (10⁶) are electroporated with 1 μg of RNA (in 50 μl) encoding the WT 1G4 alpha chain and the WT 1G4 beta chain or a modified 1G4 beta chain having an AAS within amino acids 94-96 of the 1G4 beta chain (which corresponds to the sequence VGN of the mature WT 1G4 beta chain) as indicated in Table 7. Two hours later, 10⁵ transfected T cells are added to 10⁵ C1RA2 target cells pulsed with either control gp100:154 peptide or the NY-ESO-1 HLA-A2-restricted peptide (SLLMWITQC; SEQ ID NO: 120). The release of IFN-γ by the T cells was measured on the following day. The IFN-γ released (pg/ml) by the T cells is shown in Table 7.

TABLE 7

| | C1RA2 target cells pulsed with: | | | | |
|---|---|---|---|---|---|
| WT Sequence of | 1 μM | 1 μM | 10 nM | 0.1 nM | T cells |
| β 94-96 = VGN | gp154 | ESO | ESO | ESO | alone |
| Tranfected [symbol] chain | Transfected α chain: WT | | | | |
| WT β | 71 | 1709 | 218 | 10 | 0 |
| AGN | 17 | 85 | 169 | 129 | 0 |
| LGN | 100 | 664 | 38 | 46 | 0 |
| YGN | 113 | 311 | 355 | 131 | 0 |
| EGN | 11 | 0 | 24 | 6 | 0 |
| KGN | 41 | 21 | 210 | 246 | 0 |
| PGN | 46 | 36 | 90 | 75 | 0 |
| VAN | 53 | 126 | 78 | 125 | 0 |
| VDN | 98 | 12 | 52 | 48 | 0 |
| VLN | 50 | 8 | 70 | 97 | 0 |
| VKN | 20 | 35 | 0 | 3 | 0 |
| VPN | 13 | 35 | 131 | 224 | 0 |
| VGA | 8 | 632 | 18 | 3 | 0 |
| VGD | 26 | 515 | 142 | 42 | 0 |
| VGL | 194 | 1930 | 96 | 23 | 0 |
| VGK | 10 | 5 | 79 | 0 | 0 |
| VGP | 90 | 253 | 98 | 72 | 0 |
| GFP | 157 | 67 | 52 | 48 | 0 |

The data in Table 7 demonstrates that the majority of changes made in the CDR3 of the beta chain lead to a diminished T cell response, although the modified 1G4 TCR with the VGL mutation demonstrates an enhanced T cell response.

This example demonstrated the activity of modified TCRs comprising mutations in the CDR3 of the beta chain of the 1G4 TCR.

### Example 7

This example demonstrates the biological activity of modified TCRs of the invention.

Mutations in the CDR3 of the alpha chain of the 1G4 TCR are made and tested. Experiments are performed as essentially described in previous Examples. Briefly, a pheresis sample is stimulated for 3 days with OKT3+IL-2. CD8+ T cells are isolated using anti-CD8 beads. Cells are transfected on day 5 post-stimulation with 1 μg of RNA (in a 50 ml volume) encoding WT 1G4 beta chain and WT 1G4 alpha chain or a modified version thereof in which one or more of amino acids 94-97 are modified (which correspond to the sequence PTSG (SEQ ID NO: 26) in the WT alpha chain) as indicated in Table 8. The transfected cells are stimulated with C1R-A2 target cells pulsed with the peptide indicated in Table 8 and the amount of IFNγ is measured.

TABLE 8

| | C1R-A2 target cells pulsed with | | | | |
|---|---|---|---|---|---|
| WT Sequence of | gp 154 | ESO | ESO | ESO | T cells |
| α 94-97 = PTSG | (1uM) | (1 uM) | (10 nM) | (0.1 nM) | Alone |
| Transfected [symbol] chain | Transfected beta chain: WT | | | | |
| WT α | 157 | 3718 | 450 | 147 | <10 |
| ATSG | 172 | 146 | 73 | 50 | <10 |
| HTSG | 115 | 778 | 271 | 55 | <10 |
| LTSG | 105 | 1647 | 159 | 97 | <10 |
| YTSG | 76 | 410 | 228 | 49 | <10 |
| ETSG | 118 | 576 | 144 | 91 | <10 |
| KTSG | 110 | 142 | 164 | 168 | <10 |
| PASG | 77 | 7760 | 478 | 87 | <10 |
| PLSG | 86 | 7174 | 606 | 106 | <10 |
| PYSG | 89 | 152 | 59 | 41 | <10 |
| PESG | 72 | 8824 | 432 | 133 | <10 |
| PKSG | 160 | 1892 | 62 | 70 | <10 |
| PPSG | 44 | 79 | 100 | 85 | <10 |
| PTAG | 96 | 4659 | 142 | 70 | <10 |
| PTLG | 76 | 5042 | 124 | 100 | <10 |
| PTYG | 203 | 6496 | 358 | 48 | <10 |
| PTEG | 47 | 6500 | 322 | 56 | <10 |
| PTKG | 62 | 7914 | 231 | 61 | <10 |
| PTPG | 86 | 61 | 65 | 63 | <10 |
| PTSA | 67 | 93 | 123 | 87 | <10 |
| PTSN | 39 | 131 | 55 | 73 | <10 |
| PTSL | 170 | 93 | 77 | 158 | <10 |
| PTSY | 124 | 113 | 137 | 230 | <10 |
| PTSE | 121 | 66 | 123 | 172 | <10 |
| PTSK | 166 | 217 | 233 | 190 | <10 |
| PTSP | 185 | 169 | 125 | 114 | <10 |
| GFP | 159 | 144 | 83 | 183 | <10 |
| TE8 clone | <10 | 995 | 421 | <10 | <10 |

As shown in Table 8, the alterations of the Pro at position 94 or the Gly at position 97 lead to diminished T cell recognition, whereas substitution of the Thr at position 95 with Ala, Leu, or Glu or substitution of the Ser at position 96 with Ala, Leu, Tyr, Glu, or Lys lead to enhanced T cell recognition.

This example demonstrated the biological activity of modified TCRs with AAS in the CDR3 of the alpha chain.

### Example 8

This example demonstrates the enhanced biological activity of modified TCRs of the invention.

Experiments are performed as essentially described in previous examples. Briefly, T cells are stimulated with OKT3 and subsequently selected for CD8+ T cells with anti-CD8 beads. Twelve days post-stimulation, CD8+ T cells are transfected with RNA encoding WT 1G4 alpha chain in combination with WT beta chain or a modified version thereof or WT 1G4 beta chain in combination with a WT alpha chain or a modified version thereof, as indicated in Table 9. Transfected cells are stimulated with target cells pulsed with the peptide and amount as indicated in Table 9. IFN-γ (pg/ml) released by the cells is subsequently measured and the results are set forth in Table 9.

TABLE 9

| Peptide Pulsed | gp154 | ESO | ESO | ESO | ESO |
|---|---|---|---|---|---|
| Amount of Peptide | 1 μM | 100 nM | 10 nM | 1 nM | 0.1 nM |
| Pulsed | | | | | |
| WT Sequence of | WT β | | | | |
| α51-53 = QSS | | | | | |
| WT α | 190 | 11650 | 3760 | 1080 | 220 |
| TSW | 360 | 18700 | 7900 | 2960 | 257 |
| QPW | 390 | 17000 | 9300 | 2390 | 900 |
| TFS | 430 | 7600 | 3550 | 990 | 203 |
| PFW | 2760 | 19400 | 16300 | 5180 | 3970 |
| TPW | 190 | 15300 | 9450 | 2320 | 352 |
| WT Sequence of | WT α | | | | |
| β50-53 = GAGI | | | | | |
| AAGI | 280 | 18700 | 7200 | 3200 | 397 |
| GIGI | 580 | 14400 | 17200 | 4900 | 311 |
| GAQI | 220 | 5200 | 550 | 170 | 177 |
| GAGT | 900 | 20500 | 8150 | 3520 | 281 |
| AIGI | 800 | 25600 | 11900 | 3730 | 397 |
| AAQI | 450 | 11400 | 3150 | 1370 | 147 |
| AAGT | 200 | 18600 | 7600 | 2100 | 360 |
| GIQI | 710 | 23200 | 4600 | 1790 | 276 |
| GIGT | 880 | 18900 | 4500 | 2750 | 763 |
| GAQT | 140 | 14000 | 6150 | 2300 | 294 |
| AIQI | 730 | 15000 | 7150 | 2100 | 401 |
| AIGT | 2950 | 10000 | 5700 | 3960 | 3400 |
| AAQT | 240 | 9250 | 3200 | 1330 | 249 |
| GIQT | 340 | 19000 | 12300 | 2480 | 560 |
| AIQT | 1600 | 9750 | 3600 | 2000 | 754 |
| GFP | 130 | 130 | 170 | 170 | 108 |
| WT Sequence | WT β | | | | |
| of α 95-96 = TS | | | | | |
| LY | 150 | 22600 | 13400 | 3830 | 392 |

As shown in Table 9, the mutants of the CDR2 sequence QSS (amino acids 51-53 of the wild-type 1G4 alpha chain) that appear to confer higher peptide recognition include TSW, QPW, and TPW. Also, multiple mutants of the CDR2 beta sequence GAGI (SEQ ID NO: 17; amino acids 50-53 of the WT 1G4 beta chain) also confer higher peptide recognition. Furthermore, mutation of the CDR3 alpha sequence TS (amino acids 95-96 of WT 1G4 alpha chain) to LY confers higher peptide recognition.

This example demonstrated the biological activity of modified TCRs of the invention.

### Example 9

This example demonstrates the biological activity of modified TCRs.

Multiple beta chain CDR2 mutants, the alpha chain CDR2 mutant comprising the amino acid sequence TPW, and the alpha chain CDR3 mutant comprising the amino acid sequence LY are assayed for tumor and peptide recognition.

Experiments are performed as essentially described in previous examples. Briefly, a pheresis sample is stimulated with OKT3+IL-2 for 3 days. CD8+ T cells are positively selected using magnetic beads. Five days post-stimulation, T cells (2×10⁶) are transfected with 2 μg of RNA (in 0.1 ml) encoding WT 1G4 alpha chain in combination with a modified beta chain or a WT 1G4 beta chain in combination with a modified alpha chain, as indicated in Table 10. Two hours following transfection, 10⁵ T cells are added to either 10⁵ peptide-pulsed C1RA2 EBV B cells or tumor cells having the indicated phenotype. The cells are subsequently assayed for IFN-γ release (pg/ml) and the results of the assay are shown in Table 10.

TABLE 10

| WT alpha sequence | | | QSS | TS | | | | | | | | |
|---|---|---|---|---|---|---|---|---|---|---|---|---|
| | | | (51-53) | (94-95) | | | | | | | | |
| WT beta sequence | GAGI (51-54) | | | | | | | | | | | |
| Transfected Alpha Chain | WT | WT | TPW | LY | WT | WT | WT | WT | WT | WT | WT | GFP |
| | alpha | alpha | | | alpha | alpha | alpha | alpha | alpha | alpha | alpha | |
| Transfected Beta Chain | WT | WT | WT | WT beta | AAGI | GIGI | GAQI | GAGT | AIGI | GIQT | AIQT | |
| | beta | beta | beta | | | | | | | | | |
| Target Cells + Peptide | | | | | | | | | | | | |
| C1RA2 + 1 μM g154 | 370 | 230 | 400 | 200 | 230 | 460 | 383 | 250 | 500 | 290 | 2130 | 270 |
| C1RA2 + 100 nM ESO | 3300 | 3900 | 12300 | 3270 | 3780 | 3740 | 1780 | 6350 | 6210 | 3740 | 2650 | 320 |
| C1RA2 + 10 nM ESO | 1100 | 1150 | 1970 | 1300 | 1440 | 1330 | 500 | 4480 | 2110 | 1270 | 1380 | 350 |
| C1RA2 + 1 nM ESO | 322 | 300 | 470 | 670 | 480 | 610 | 350 | 420 | 560 | 1000 | 1240 | 310 |
| C1RA2 + 0.1 nM ESO | 326 | 150 | 380 | 560 | 330 | 550 | 360 | 280 | 270 | 460 | 1200 | 250 |
| Tumor Cells (Phenotype) | | | | | | | | | | | | |
| A375 (HLA-A2+/ESO+) | 90 | 50 | 240 | 160 | 120 | 180 | 100 | 240 | 140 | 120 | 80 | 50 |
| 624.38 (HLA-A2+/ESO+) | 140 | 110 | 600 | 260 | 610 | 880 | 300 | 900 | 300 | 180 | 210 | <10 |
| 1363 (HLA-A2+/ESO+) | 720 | 1160 | 1480 | 1140 | 1520 | 2280 | 320 | 2590 | 1560 | 760 | 40 | <10 |
| 1390 (HLA-A2+/ESO+) | 270 | 200 | 4770 | 2100 | 1550 | 3010 | 110 | 4030 | 3100 | 1560 | 120 | <10 |
| SK23 (HLA-A2+/ESO−) | <10 | <10 | 50 | 60 | 60 | 80 | 20 | <10 | <10 | <10 | 120 | 30 |
| 526 (HLA-A2+/ESO−) | 40 | <10 | 110 | 100 | 90 | 70 | 80 | <10 | <10 | <10 | 350 | 30 |
| 1102 (HLA-A2+/ESO−) | <10 | <10 | <10 | <10 | <10 | <10 | <10 | <10 | <10 | <10 | 120 | <10 |
| 1910 (HLA-A2+/ESO−) | <10 | <10 | <10 | <10 | <10 | <10 | <10 | <10 | <10 | <10 | <10 | <10 |
| 1359 (HLA-A2−/ESO+) | <10 | <10 | <10 | <10 | <10 | <10 | <10 | <10 | <10 | <10 | <10 | <10 |
| Alone | 70 | <10 | 80 | 90 | 80 | 90 | 80 | 10 | <10 | <10 | 20 | 70 |

As shown in Table 10, the modified TCR chains comprising TPW, AAGI, GIGI, GAGT, or AIGI exhibit enhanced biological activity.

### Example 10

This example demonstrates the additional comparison between alpha chain and beta chain TCR mutants.

Experiments are performed as essentially described in previous examples. Briefly, T cells are stimulated with OKT3. After three days, cells are separated using anti-CD8 beads, such that >99% are CD8 positive. Thirteen days post-stimulation, the CD8+ T cells (2×10⁶) are electroporated with 2 μg of RNA encoding WT or mutant alpha and beta chains as indicated in Tables 11 and 12. Two hours later, 10⁵ transfected T cells are incubated with 10⁵ C1R-A2 cells pulsed with control peptide or native NY-ESO-1 peptide or are incubated with tumor cell lines (10⁵ except for 1390 mel, only 2.5×10⁴ are used) overnight. IFN-γ release is measured on the following day. The results of the IFN-γ release are shown in Tables 11 and 12.

TABLE 11

| WT alpha sequence | | QSS | TS | | | | | | |
|---|---|---|---|---|---|---|---|---|---|
| | | (aa 51-53) | (aa 95-96) | | | | | | |
| WT beta sequence | GAGI (aa 51-54) | | | | | | | | |
| Transfected alpha chain | WT | TPW | LY | WT | WT | WT | WT | WT | |
| | | (aa 51-53) | (aa 94-96) | | | | | | |
| Transfected beta chain | WT | WT | WT | AAGI | GIGI | GAGT | AIGI | GIQT | |
| | | | | (aa 51-54) | (aa 51-54) | (aa 51-54) | (aa 51-54) | (aa 51-54) | |
| Target Cells + Peptide | | | | | | | | | |
| C1RA2 + 1 μM g154 | 42 | 76 | 67 | 60 | 44 | 52 | 89 | 190 | 33 |
| C1RA2 + 100 nM ESO | 1436 | 1188 | 2130 | 1997 | 1801 | 2881 | 1714 | 2402 | 36 |
| C1RA2 + 10 nM ESO | 540 | 2009 | 2138 | 871 | 1764 | 1939 | 941 | 1343 | 37 |
| C1RA2 + 1 nM ESO | 128 | 805 | 635 | 346 | 509 | 564 | 675 | 740 | 34 |
| C1RA2 + 0.1 nM ESO | 72 | 244 | 169 | 127 | 134 | 134 | 213 | 267 | 30 |
| Tumor Cells (Phenotype) | | | | | | | | | |
| A375 (A2+/ESO+) | 10 | 275 | 128 | 64 | 153 | 143 | 93 | 46 | <10 |
| 624.38 (A2+/ESO+) | 130 | 743 | 527 | 455 | 560 | 524 | 452 | 344 | <10 |
| 1363 (A2+/ESO+) | 183 | 790 | 806 | 562 | 881 | 767 | 625 | 485 | <10 |
| 1390 (A2+/ESO+) | 134 | 1172 | 1051 | 750 | 1196 | 1161 | 1128 | 1051 | <10 |
| 1359-A2 (A2+/ESO+) | 375 | 627 | 324 | 230 | 441 | 177 | 310 | 187 | <10 |
| 1359 (A2−/ESO+) | <10 | <10 | <10 | <10 | <10 | <10 | <10 | <10 | <10 |
| SK23 (A2+/ESO−) | <10 | <10 | <10 | <10 | <10 | <10 | <10 | <10 | <10 |
| 526 (A2−/ESO−) | <10 | <10 | <10 | <10 | <10 | <10 | <10 | <10 | <10 |
| 1102 (A2+/ESO−) | <10 | 12 | <10 | <10 | <10 | 12 | <10 | 22 | <10 |
| T alone | 27 | 23 | 26 | 26 | 18 | 23 | 26 | 22 | 22 |

TABLE 12

| WT alpha sequence | QSS (aa 51-53) | | | TS (aa 95-96) | | | |
|---|---|---|---|---|---|---|---|
| WT beta sequence | GAGI (aa 51-54) | | | | | | |
| Transfected alpha chain | TPW (aa 51-53) | | | LY (aa95-96) | | | WT |
| Transfected beta chain | GIGI | GAGT | AIGI | GIGI | GAGT | AIGI | WT |
| | (aa 51-54) | (aa 51-54) | (aa 51-54) | (aa 51-54) | (aa 51-54) | (aa 51-54) | |
| Peptide Pulsed Target Cells | | | | | | | |
| C1RA2 + 1 μM g154 | 372 | 268 | 469 | 72 | 65 | 192 | 30 |
| C1RA2 + 100 nM ESO | 2732 | 3476 | 3105 | 3816 | 2240 | 2751 | 927 |
| C1RA2 + 10 nM ESO | 3039 | 2476 | 1561 | 2380 | 1994 | 659 | 538 |
| C1RA2 + 1 nM ESO | 1015 | 472 | 913 | 1136 | 574 | 380 | 105 |
| C1RA2 + 0.1 nM ESO | 367 | 414 | 761 | 239 | 187 | 260 | 59 |
| T alone | 24 | 26 | 23 | 19 | 23 | 17 | 20 |
| Tumor Cells (Phenotype) | | | | | | | |
| A375 (A2+/ESO+) | 57 | 29 | 14 | 88 | 53 | <10 | <10 |
| 624.38 (A2+/ESO+) | 355 | 201 | 167 | 525 | 208 | 30 | 30 |
| 1363 (A2+/ESO+) | 517 | 341 | 120 | 625 | 380 | 71 | 83 |
| 1390 (A2+/ESO+) | 1119 | 1003 | 513 | 1370 | 1050 | 389 | 114 |
| 1359-A2 (A2+/ESO+) | 464 | 346 | 240 | 706 | 292 | 361 | 30 |
| 1359 (A2−/ESO+) | <10 | <10 | <10 | <10 | <10 | <10 | <10 |
| SK23 A2+/ESO−) | <10 | <10 | 23 | <10 | <10 | <10 | <10 |
| 526 (A2−/ESO−) | <10 | <10 | 27 | <10 | <10 | <10 | <10 |
| 1102 (A2+/ESO−) | 90 | 99 | 149 | <10 | <10 | <10 | <10 |

As shown in Tables 11 and 12, mutated TCRs confer CD8+ T cells with enhanced recognition of tumor targets. Also, combinations of mutated alpha with mutated beta constructs can result in diminished recognition (LY (amino acids 95-96) plus AIGI (amino acids 50-53) mutants) as compared to the TCR comprising the LY mutant and WT beta chain or the TCR comprising the AIGI mutant and the WT alpha chain. However, the activity of the TCR comprising both mutated alpha and beta chains is still enhanced as compared to the WT TCR. Further, the TCR comprising the mutated alpha and beta chains comprising LY (amino acids 95-96) or GIGI (amino acids 50-53) demonstrates enhanced activity as compared to WT.

This example demonstrated the biological activity of modified TCRs of the invention.

### Example 11

This example demonstrates yet more comparisons of transfected CD4 and CD8 T cells expressing modified TCRs of the invention.

Experiments are carried out as essentially described in previous examples. Briefly, T cells are stimulated with OKT3 for four days and then separated using anti-CD4 beads on an Milteyni LS column. The cells that flow through the column are passed over an LD column to obtain CD8+ T cells. The CD4+ T cells are >98% pure, while the CD8+ T cells are >90% pure. Cell populations are transfected with RNA encoding the indicated TCR chains as indicated in Tables 13 and 14. Transfected T cells (10⁵ CD8+ T cells or 6×10⁴ CD4+ T cells) are stimulated with the indicated target cells (10⁵). Negative control target cells include 1102 cells and 1088 cells, both of which are HLA-A2+) and 888 cells, which are HLA-A2 negative cells. IFN release assays are performed the next day. The results of the assays are shown in Tables 13 (CD8+ T cells) and 14 (CD4+ T cells).

TABLE 13

| WT alpha sequence | | TS | | | | |
|---|---|---|---|---|---|---|
| | | (aa 95-96) | | | | |
| WT beta sequence | GAGI (51-54) | | | | | |
| Transfected Alpha Chain | WT | LY | WT | WT | WT | GFP |
| Transfected Beta Chain | WT | WT | GIGI | AIGI | AIQT | |
| Target Cells + Peptide | | | | | | |
| T2 + g154 | 73 | 126 | 53 | 98 | 660 | 833 |
| T2 + 10 nM ESO | 3200 | 7370 | 4010 | 4290 | 1810 | 764 |
| T2 + 1 nM ESO | 552 | 2300 | 1163 | 1215 | 701 | 726 |
| T2 + 0.1 nM ESO | 131 | 510 | 278 | 281 | 667 | 568 |
| Tumor Cells (Phenotype) | | | | | | |
| 1363 mel (A2+/ESO+) | 633 | 4075 | 996 | 786 | 37 | 60 |
| 1390 mel (A2+/ESO+) | 182 | 1707 | 704 | 627 | 45 | 42 |
| 624.38 mel (A2+/ESO+) | 241 | 720 | 317 | 216 | 42 | 8 |
| 1359 mel (A2−/ESO+) | <30 | <30 | <30 | <30 | <30 | 13 |
| 1359A2 mel | 200 | 1066 | 324 | 373 | 39 | 22 |
| (A2+/ESO+) | | | | | | |
| 1102 mel (A2+/ESO−) | <30 | <30 | <30 | <30 | <30 | 16 |
| 526 mel (A2+/ESO−) | <30 | <30 | <30 | <30 | 43 | 5 |
| 1102 EBV (A2+/ESO−) | 200 | 2160 | 1170 | 1980 | 263 | 81 |
| 1088 EBV (A2+/ESO−) | 529 | 379 | 338 | 294 | 507 | 369 |
| 888 EBV (A2−/ESO+) | 15 | 74 | 110 | 54 | 26 | 36 |
| T alone | <30 | <30 | <30 | <30 | <30 | 15 |

TABLE 14

| WT alpha sequence | | TS | | | | |
|---|---|---|---|---|---|---|
| | | (95-96) | | | | |
| WT beta sequence | GAGI (50-53) | | | | | |
| Transfected Alpha Chain | WT | PLY | WT | WT | WT | GFP |
| Transfected Beta Chain | WT | WT | GIGI | AIGI | AIQT | |
| Target Cells + Peptide | | | | | | |
| T2 + g154 | 447 | 795 | 638 | 780 | 4050 | 833 |
| T2 + 10 nM ESO | 951 | 8700 | 14000 | 21700 | 11810 | 764 |
| T2 + 1 nM ESO | 614 | 6530 | 6280 | 11310 | 6710 | 726 |
| T2 + 0.1 nM ESO | 488 | 2680 | 2740 | 5320 | 5190 | 568 |
| Tumor Cells (Phenotype) | | | | | | |
| 1363 mel (A2+/ESO+) | 55 | 932 | 1570 | 5820 | 694 | 60 |
| 1390 mel (A2+/ESO+) | <30 | 883 | 1182 | 7820 | 2560 | 42 |
| 624.38 mel (A2+/ESO+) | <30 | 82 | 162 | 939 | 25 | 8 |
| 1359 mel (A2−/ESO+) | <30 | 137 | 195 | 1306 | 259 | 22 |
| 1359A2 mel (A2+/ESO+) | <30 | <30 | <30 | <30 | <30 | 13 |
| 1102 mel (A2+/ESO−) | <30 | <30 | <30 | <30 | <30 | 16 |
| 526 mel (A2+/ESO−) | <30 | <30 | <30 | <30 | <30 | <30 |
| 1102 EBV (A2+/ESO−) | 491 | 1760 | 1680 | 9190 | 2740 | 351 |
| 1088 EBV (A2+/ESO−) | 1431 | 1020 | 1200 | 1358 | 1685 | 731 |
| 888 EBV (A2−/ESO+) | 687 | 1073 | 758 | 513 | 499 | 595 |
| T alone | <30 | <30 | <30 | <30 | <30 | <30 |

As shown in Tables 13 and 14, the modified TCRs comprising the sequence LY, GIGI, or AIGI exhibit enhanced T cell recognition.

This example demonstrated the enhanced T cell activity of modified TCRs.

### Example 12

This example demonstrates the biological activity of the modified TCRs of the invention.

Experiments are carried out as essentially described in previous examples. CD4+ or CD8+ T cells are transfected on Day 6 post-OKT3 stimulation with RNA encoding WT F5 alpha chain in combination with WT F5 beta chain or a modified version thereof having an AAS within amino acids 51-54 (which corresponds to the sequence TAGT (SEQ ID NO: 40) of the WT F5 beta chain). The results of the IFN release assays are shown in Tables 15 (CD8+ T cells) and 16 (CD4+ T cells).

TABLE 15

| beta chain: | WT | TAGA | VAGT | TGGT | TIGT | TMGT |
|---|---|---|---|---|---|---|
| | | (T54A) | (T51V) | (A52G) | (A52I) | (A52M) |
| Target Cells + Peptide | | | | | | |
| T2 + gp100 | 41 | <30 | <30 | 7.61 | 32 | <30 |
| T2 + 1 μM MART-1 | 15410 | 22 | 9260 | 5680 | 11420 | 9790 |
| Tumor Cells (Phenotype) | | | | | | |
| A375 (A2+/MART−) | <30 | <30 | <30 | <30 | <30 | <30 |
| 397-A2 (A2+/MART+) | 18190 | <30 | 17240 | 2680 | 15530 | 20740 |
| 397-A24 (A2−/MART+) | <30 | <30 | <30 | <30 | <30 | <30 |
| 624.28 (A2+/MART−) | <30 | <30 | <30 | <30 | <30 | <30 |
| 624.38 (A2+/MART+) | 8620 | <30 | 9100 | 1730 | 6890 | 8300 |
| 1300 (A2+/MART+) | 42050 | 553 | 33360 | 19927 | 36201 | 43546 |
| SK (A2+/MART+) | 16410 | <30 | 14070 | 2760 | 14060 | 11320 |
| alone | <30 | <30 | <30 | <30 | <30 | <30 |

TABLE 16

| | WT | TAGA | VAGT | TGGT | TIGT | TMGT |
|---|---|---|---|---|---|---|
| | | (T54A) | (T51V) | (A52G) | (A52I) | (A52M) |
| Target Cells + Peptide | | | | | | |
| T2 + gp100 | <30 | <30 | <30 | <30 | <30 | <30 |
| T2 + 1 μM Mart | 29114 | <30 | 60101 | 36 | 25785 | 25785 |
| Tumor Cells (Phenotype) | | | | | | |
| A375 (MART−/A2+) | <30 | <30 | <30 | <30 | <30 | <30 |
| 397-A2 (MART+/A2+) | 15581 | <30 | 18714 | <30 | 13599 | 21137 |
| 397-A24 (MART+/A2−) | 64 | <30 | <30 | <30 | <30 | <30 |
| 624.28 (MART−/A2+) | <30 | <30 | <30 | <30 | <30 | <30 |
| 624.38 (MART+/A2+) | 3920 | <30 | 15280 | <30 | 7300 | 8930 |
| 1300 (MART+/A2+) | 22002 | 43 | 36146 | 173 | 21424 | 28595 |
| SK23 (MART+/A2+) | 9688 | <30 | 21040 | 35 | 13987 | 13875 |
| alone | <30 | <30 | <30 | <30 | <30 | <30 |

As shown in Tables 15 and 16, the modified TCRs comprising the modified sequence of VAGT or TMGT exhibit enhanced T cell reactivity.

This example demonstrated the biological activity of modified TCRs.

### Example 13

This example demonstrates yet more comparisons of transfected CD4 and CD8 T cells expressing modified TCRs of the invention.

Experiments are carried out as essentially described in previous examples. CD4+ T cells or CD8+ T cells are transfected on Day 6 post-OKT3 stimulation with RNA encoding WT F5 alpha chain in combination with WT F5 beta chain or a modified version thereof having an AAS within amino acids 51-54 (which corresponds to the sequence TAGT (SEQ ID NO: 40) of the WT F5 beta chain). The results of the cytokine release assays are shown in Tables 17 (CD8+ T cells) and 18 (CD4+ T cells).

TABLE 17

| Modified AA Sequence | LAGT | MAGT | VAGT | TGGT | TIGT | TLGT | TMGT | TSGT | TTGT | |
|---|---|---|---|---|---|---|---|---|---|---|
| AAS | 70L | 70M | 70V | 71G | 71I | 71L | 71M | 71S | 71T | WT |
| Target Cells + Peptide | | | | | | | | | | |
| T2 + gp100 | <30 | <30 | 260 | 38 | <30 | <30 | <30 | <30 | <30 | <30 |
| T2 + MART 1 μM | 15480 | 17340 | 20340 | 6710 | <30 | 16190 | 14160 | 72 | <30 | 17910 |
| Tumor Cells (Phenotype) | | | | | | | | | | |
| A375 (MART−/A2+) | 150 | 106 | 155 | 113 | <30 | 115 | 124 | 81 | 73 | 66 |
| 397-A2 (MART+/A2+) | 3210 | 2610 | 3990 | 510 | 75 | 3170 | 3300 | 69 | 48 | 3520 |
| 397-A24 (MART+/A2−) | <30 | <30 | <30 | <30 | <30 | <30 | <30 | <30 | <30 | <30 |
| 624.28 (MART−/A2+) | 208 | 153 | 262 | 177 | <30 | 171 | 217 | 163 | 128 | 111 |
| 624.38 (MART+/A2+) | 3470 | 3310 | 3270 | 730 | <30 | 3250 | 3280 | 103 | 67 | 3360 |
| Alone | <30 | <30 | <30 | <30 | <30 | <30 | <30 | <30 | <30 | <30 |

TABLE 18

| Modified AA Sequence | LAGT | MAGT | VAGT | TGGT | TIGT | TLGT | TMGT | TSGT | TTGT | |
|---|---|---|---|---|---|---|---|---|---|---|
| AAS | 70L | 70M | 70V | 71G | 71I | 71L | 71M | 71S | 71T | WT |
| Target Cells + Peptide | | | | | | | | | | |
| T2 + gp100 | <30 | <30 | <30 | <30 | <30 | <30 | <30 | <30 | <30 | <30 |
| T2 + MART 1 μM | 916 | 838 | 10740 | 16 | 10030 | 5700 | 9570 | <30 | 4 | 6310 |
| Tumor Cells (Phenotype) | | | | | | | | | | |
| A375 (MART−/A2+) | 163 | 116 | 156 | 150 | 146 | 158 | 142 | 158 | 142 | 156 |
| 397-A2 (MART+/A2+) | 87 | 350 | 2910 | 11 | 1680 | 419 | 1690 | 11 | <30 | 488 |
| 397-A24 (MART+/A2−) | <30 | <30 | <30 | <30 | <30 | <30 | <30 | <30 | <30 | <30 |
| 624.28 (MART−/A2+) | <30 | <30 | <30 | <30 | <30 | <30 | 241 | <30 | <30 | 46 |
| 624.38 (MART+/A2+) | 66 | 57 | 1960 | <30 | 906 | 239 | 2442 | <30 | <30 | 901 |
| Alone | 247 | <30 | <30 | <30 | <30 | <30 | <30 | <30 | <30 | <30 |

As shown in Tables 17 and 18, the modified TCRs comprising the modified sequence VAGT or TMGT lead to enhanced T cell reactivity.

This example demonstrated the identification of modified TCRs of the invention.

### Example 14

This example demonstrates a method of treating a disease in a host using the inventive TCRs.

Adoptive cell transfer is carried out as described in Morgan et al. (2006), supra. Briefly, PBLs are obtained by leukopheresis from a metastatic melanoma patient who is HLA-A*0201 positive. The PBLs are transduced with nucleic acids encoding a WT alpha chain and a modified beta chain of a TCR specific for either NY-ESO-1 or MART-1 as described in Example 1. The patient receives the transduced cells at the time of maximum lymphodepletion. One month post-adoptive cell transfer, quantitative RT-PCR assays are carried out to reveal whether the presence of the modified TCRs are expressed by cells of the patient. Tumor regression also is analyzed by the methods described in Morgan et al. (2006), supra.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated protein comprising an amino acid sequence selected from: the group consisting of
(i) SEQ ID NO: 127, wherein Xaa at positions 51-53 and 96-98 are wild-type amino acids, wherein Xaa at position 54 is Thr;
(ii) SEQ ID NO: 127, wherein Xaa at positions 51 and 96-98 are wild-type amino acids, wherein Xaa at positions 52-54 are Ile, Gln, and Ile, respectively;
(iii) SEQ ID NO: 127, wherein Xaa at positions 51, 53, and 96-98 are wild-type amino acids, wherein Xaa at positions 52 and 54 are Ile and Thr, respectively;
(iv) SEQ ID NO: 127, wherein Xaa at positions 51, 52, and 96-98 are wild-type amino acids, wherein Xaa at positions 53 and 54 are Gln and Thr, respectively;
(v) SEQ ID NO: 127, wherein Xaa at positions 54 and 96-98 are wild-type amino acids, wherein Xaa at positions 51-53 are Ala, Ile, Gln, respectively;
(vi) SEQ ID NO: 127, wherein Xaa at positions 53 and 96-98 are wild-type amino acids, wherein Xaa at positions 51, 52, and 54 are Ala, Ile, and Thr, respectively;
(vii) SEQ ID NO: 127, wherein Xaa at positions 51 and 96-98 are wild-type amino acids, wherein Xaa at positions 52-54 are Ile, Gln, and Thr, respectively;
(viii) SEQ ID NO: 127, wherein Xaa at positions 96-98 are wild-type amino acids, wherein Xaa at positions 51-54 are Ala, Ile, Gln, and Thr respectively;
(ix) SEQ ID NO: 127, wherein Xaa at positions 51-54, 96, and 97 are wild-type amino acids, wherein Xaa at position 98 is Leu;
(x) SEQ ID NO: 128, wherein Xaa at positions 94-97 are wild-type amino acids, wherein Xaa at positions 51-53 are Thr, Pro, and Trp, respectively;
(xi) SEQ ID NO: 128, wherein Xaa at positions 52, 53, and 94-97 are wild-type amino acids, wherein Xaa at position 51 is Pro;
(xii) SEQ ID NO: 128, wherein Xaa at positions 52, 53, and 94-97 are wild-type amino acids, wherein Xaa at position 51 is Thr;
(xiii) SEQ ID NO: 128, wherein Xaa at positions 51, 53, and 94-97 are wild-type amino acids, wherein Xaa at position 52 is Phe;
(xiv) SEQ ID NO: 128, wherein Xaa at positions 51, 53, and 94-97 are wild-type amino acids, wherein Xaa at position 52 is Pro;
(xv) SEQ ID NO: 128, wherein Xaa at positions 51, 52, and 94-97 are wild-type amino acids, wherein Xaa at position 53 is Trp;
(xvi) SEQ ID NO: 128, wherein Xaa at positions 53 and 94-97 are wild-type amino acids, wherein Xaa at positions 51 and 52 are Pro and Phe, respectively;
(xvii) SEQ ID NO: 128, wherein Xaa at positions 52 and 94-97 are wild-type amino acids, wherein Xaa at positions 51 and 53 are Pro and Trp;
(xviii) SEQ ID NO: 128, wherein Xaa at positions 51 and 94-97 are wild-type amino acids, wherein Xaa at positions 52 and 53 are Phe and Trp;
(xix) SEQ ID NO: 128, wherein Xaa at positions 53 and 94-97 are wild-type amino acids, wherein each Xaa at positions 51 and 52 is Pro, respectively;
(xx) SEQ ID NO: 128, wherein Xaa at positions 53 and 94-97 are wild-type amino acids, wherein Xaa at positions 51 and 52 is Thr and Pro, respectively;
(xxi) SEQ ID NO: 128, wherein Xaa at positions 52 and 94-97 are wild-type amino acids, wherein Xaa at positions 51 and 53 are Thr and Trp, respectively;
(xxii) SEQ ID NO: 128, wherein Xaa at positions 51 and 94-97 are wildtype amino acids, wherein Xaa at positions 52 and 53 are Pro and Trp, respectively;
(xxiii) SEQ ID NO: 128, wherein Xaa at positions 53 and 94-97 are wild-type amino acids, wherein Xaa at positions 51 and 52 are Thr and Phe, respectively;
(xxiv) SEQ ID NO: 128, wherein Xaa at positions 94-97 are wild-type amino acids, wherein Xaa at positions 51-53 are Pro, Phe, and Trp, respectively;
(xxv) SEQ ID NO: 128, wherein Xaa at positions 51-53, 94, and 97 are wild-type amino acids, wherein Xaa at positions 95 and 96 are Leu and Tyr, respectively;
(xxvi) SEQ ID NO: 128, wherein Xaa at positions 51-53, 94, 96, and 97 are wild-type amino acids, wherein Xaa at position 95 is Ala;
(xxvii) SEQ ID NO: 128, wherein Xaa at positions 51-53, 94, 96, and 97 are wild-type amino acids, wherein Xaa at position 95 is Leu;
(xxviii) SEQ ID NO: 128, wherein Xaa at positions 51-53, 94, 96, and 97 are wild-type amino acids, wherein Xaa at position 95 is Glu;
(xxix) SEQ ID NO: 128, wherein Xaa at positions 51-53, 94, 95, and 97 are wild-type amino acids, wherein Xaa at position 96 is Ala;
(xxx) SEQ ID NO: 128, wherein Xaa at positions 51-53, 94, 95, and 97 are wild-type amino acids, wherein Xaa at position 96 is Leu;
(xxxi) SEQ ID NO: 128, wherein Xaa at positions 51-53, 94, 95, and 97 are wild-type amino acids, wherein Xaa at position 96 is Tyr;
(xxxii) SEQ ID NO: 128, wherein Xaa at positions 51-53, 94, 95, and 97 are wild-type amino acids, wherein Xaa at position 96 is Glu;
(xxxiii) SEQ ID NO: 128, wherein Xaa at positions 51-53, 94, 95, and 97 are wild-type amino acids, wherein Xaa at position 96 is Lys;
(xxxiv) SEQ ID NO: 128, wherein Xaa at positions 51-53 and 94 are wild-type amino acids, wherein Xaa at positions 95-97 are Leu, Leu, and Asp, respectively;
(xxxv) SEQ ID NO: 128, wherein Xaa at positions 51-53, 94, and 97 are wild-type amino acids, wherein each Xaa at positions 95 and 96 are Leu;
(xxxvi) SEQ ID NO: 128, wherein Xaa at positions 51-53, 94, and 97 are wild-type amino acids, wherein Xaa at positions 95 and 96 are Glu and Tyr, respectively;
(xxxvii) SEQ ID NO: 128, wherein Xaa at positions 51-53, 94, and 97 are wild-type amino acids, wherein Xaa at positions 95 and 96 are Trp and Val, respectively;
(xxxviii) SEQ ID NO: 128, wherein Xaa at positions 51-53, 96, and 97 are wild-type amino acids, wherein Xaa at positions 94 and 95 are Leu and Pro, respectively;
(xxxix) SEQ ID NO: 132, wherein Xaa at positions 52 to 54 are wild-type amino acids, wherein Xaa at position 51 is Val; and
(xxxx) SEQ ID NO: 132, wherein Xaa at positions 51, 53, and 54 are wild-type amino acids, wherein Xaa at position 52 is Met;
or a combination thereof;
wherein the wild-type amino acids of SEQ ID NO: 127 at positions 51-54 and 96-98 are Gly, Ala, Gly, Ile, Val, Gly, and Asn, respectively,
wherein the wild-type amino acids of SEQ ID NO: 128 at positions 51-53 and 94-97 are Gln, Ser, Ser, Pro, Thr, Ser, and Gly, respectively,
wherein the wild-type amino acids of SEQ ID NO: 132 at positions 51-54 are Thr, Ala, Gly, and Thr, respectively.

2. The isolated protein of claim 1, wherein, when the protein comprises an amino acid sequence selected from the group consisting of (i) to (ix), the protein further comprises SEQ ID NO: 124, when the protein comprises an amino acid sequence selected from the group consisting of (x) to (xxviii), the protein further comprises SEQ ID NO: 123, and when the protein comprises an amino acid sequence selected from the group consisting of (xxviii) and (xxix), the protein further comprises SEQ ID NO: 14, or a mature form thereof.

3. The isolated protein of claim 1, wherein, when the protein comprises the amino acid sequence of (x) or (xxv), the protein further comprises the amino acid sequence of SEQ ID NO: 127, wherein Xaa at positions 51, 53, 54, and 96-98 are wild-type amino acids and Xaa at position 52 is Ile, or SEQ ID NO: 127, wherein Xaa at positions 53, 54, and 96-98 are wild-type amino acids and Xaa at positions 51 and 52 are Ala and Ile, respectively.

4. The isolated protein of claim 1, wherein the protein comprises an amino acid sequence selected from the group consisting of
(i) SEQ ID NO: 7, wherein Xaa at positions 70-72 and 115-117 are wild-type amino acids, wherein Xaa at position 73 is Thr;
(ii) SEQ ID NO: 7, wherein Xaa at positions 70 and 115-117 are wild-type amino acids, wherein Xaa at positions 71-73 are Ile, Gln, and Ile, respectively;
(iii) SEQ ID NO: 7, wherein Xaa at positions 70, 72, and 115-117 are wild-type amino acids, wherein Xaa at positions 71 and 73 are Ile and Thr, respectively;
(iv) SEQ ID NO: 7, wherein Xaa at positions 70, 71, and 115-117 are wild-type amino acids, wherein Xaa at positions 72 and 73 are Gln and Thr, respectively;
(v) SEQ ID NO: 7, wherein Xaa at positions 73 and 115-117 are wild-type amino acids, wherein Xaa at positions 70-72 are Ala, Ile, Gln, respectively;
(vi) SEQ ID NO: 7, wherein Xaa at positions 72 and 115-117 are wild-type amino acids, wherein Xaa at positions 70, 71, and 73 are Ala, Ile, and Thr, respectively;
(vii) SEQ ID NO: 7, wherein Xaa at positions 70 and 115-117 are wild-type amino acids, wherein Xaa at positions 71-73 are Ile, Gln, and Thr, respectively;
(viii) SEQ ID NO: 7, wherein Xaa at positions 115-117 are wild-type amino acids, wherein Xaa at positions 70-73 are Ala, Ile, Gln, and Thr respectively;
(ix) SEQ ED NO: 7, wherein Xaa at positions 70-73, 115, and 116 are wild-type amino acids, wherein Xaa at position 117 is Leu;
(x) SEQ ID NO: 8, wherein Xaa at positions 113-116 are wild-type amino acids, wherein Xaa at positions 70-72 are Thr, Pro, and Trp, respectively;
(xi) SEQ ID NO: 8, wherein Xaa at positions 71, 72, and 113-116 are wild-type amino acids, wherein Xaa at position 70 is Pro;
(xii) SEQ ID NO: 8, wherein Xaa at positions 71, 72, and 113-116 are wild-type amino acids, wherein Xaa at position 70 is Thr;
(xiii) SEQ ID NO: 8, wherein Xaa at positions 70, 72, and 113-116 are wild-type amino acids, wherein Xaa at position 71 is Phe;
(xiv) SEQ ID' NO: 8, wherein Xaa at positions 70, 72, and 113-116 are wild-type amino acids, wherein Xaa at position 71 is Pro;
(xv) SEQ ID NO: 8, wherein Xaa at positions 70, 71, and 113-116 are wild-type amino acids, wherein Xaa at position 72 is Trp;
(xvi) SEQ ID NO: 8, wherein Xaa at positions 72 and 113-116 are wild-type amino acids, wherein Xaa at positions 70 and 71 are Pro and Phe, respectively;
(xvii) SEQ ID NO: 8, wherein Xaa at positions 71 and 113-116 are wild-type amino acids, wherein Xaa at positions 70 and 72 are Pro and Trp;
(xviii) SEQ ID NO: 8, wherein Xaa at positions 70 and 113-116 are wild-type amino acids, wherein Xaa at positions 71 and 72 are Phe and Trp;
(xix) SEQ ID NO: 8, wherein Xaa at positions 72 and 113-116 are wild-type amino acids, wherein each Xaa at positions 70 and 71 is Pro, respectively;
(xx) SEQ ID NO: 8, wherein Xaa at positions 72 and 113-116 are wild-type amino acids, wherein Xaa at positions 70 and 71 is Thr and Pro, respectively;
(xxi) SEQ ID NO: 8, wherein Xaa at positions 71 and 113-116 are wild-type amino acids, wherein Xaa at positions 70 and 72 are Thr and Trp, respectively;
(xxii) SEQ ID NO: 8, wherein Xaa at positions 70 and 113-116 are wildtype amino acids, wherein Xaa at positions 71 and 72 are Pro and Trp, respectively;
(xxiii) SEQ ID NO: 8, wherein Xaa at positions 72 and 113-116 are wild-type amino acids, wherein Xaa at positions 70 and 71 are Thr and Phe, respectively;
(xxiv) SEQ ID NO: 8, wherein Xaa at positions 113-116 are wild-type amino acids, wherein Xaa at positions 70 to 72 are Pro, Phe, and Trp, respectively;
(xxv) SEQ ID NO: 8, wherein Xaa at positions 70-72, 113, and 116 are wild-type amino acids, wherein Xaa at positions 114 and 115 are Leu and Tyr, respectively;
(xxvi) SEQ ID NO: 8, wherein Xaa at positions 70-72, 113, 115, and 116 are wild-type amino acids, wherein Xaa at position 114 is Ala;
(xxvii) SEQ ID NO: 8, wherein Xaa at positions 70-72, 113, 115, and 116 are wild-type amino acids, wherein Xaa at position 114 is Leu;
(xxviii) SEQ ID NO: 8, wherein Xaa at positions 70-72, 113, 115, and 116 are wild-type amino acids, wherein Xaa at position 114 is Glu;
(xxix) SEQ ID NO: 8, wherein Xaa at positions 70-72, 113, 114, and 116 are wild-type amino acids, wherein Xaa at position 115 is Ala;
(xxx) SEQ JD NO: 8, wherein Xaa at positions 70-72, 113, 114, and 116 are wild-type amino acids, wherein Xaa at position 115 is Leu;
(xxxi) SEQ ID NO: 8, wherein Xaa at positions 70-72, 113, 114, and 116 are wild-type amino acids, wherein Xaa at position 115 is Tyr;
(xxxii) SEQ ID NO: 8, wherein Xaa at positions 70-72, 113, 114, and 116 are wild-type amino acids, wherein Xaa at position 115 is Glu;
(xxxiii) SEQ ID NO: 8, wherein Xaa at positions 70-72, 113, 114, and 116 are wild-type amino acids, wherein Xaa at position 115 is Lys;
(xxxiv) SEQ ID NO: 8, wherein Xaa at positions 70-72 and 113 are wild-type amino acids, wherein Xaa at positions 114-116 are Leu, Leu, and Asp, respectively;
(xxxv) SEQ ID NO: 8, wherein Xaa at positions 70-72, 113, and 116 are wild-type amino acids, wherein each Xaa at positions 114 and 115 are Leu;
(xxxvi) SEQ ID NO: 8, wherein Xaa at positions 70-72, 113, and 116 are wild-type amino acids, wherein Xaa at positions 114 and 115 are Glu and Tyr, respectively;
(xxxvii) SEQ ID NO: 8, wherein Xaa at positions 70-72, 115, and 116 are wild-type amino acids, wherein Xaa at positions 113 and 114 are Leu and Pro, respectively;
(xxxviii) SEQ ID NO: 12, wherein Xaa at positions 71 to 73 are wild-type amino acids, wherein Xaa at position 70 is Val; and
(xxxix) SEQ ID NO: 12, wherein Xaa at positions 70, 72, and 73 are wild-type amino acids, wherein Xaa at position 71 is Met;
or a combination thereof;
wherein the wild-type amino acids of SEQ ID NO: 7 at positions 70-73 and 115-117 are Gly, Ala, Gly, Ile, Val, Gly, and Asn, respectively,
wherein the wild-type amino acids of SEQ ID NO: 8 at positions 70-72 and 113-116 are Gln, Ser, Ser, Pro, Thr, Ser, and Gly, respectively,
wherein the wild-type amino acids of SEQ ID NO: 12 at positions 70-73 are Thr, Ala, Gly, and Thr, respectively.

5. The isolated protein of claim 1 comprising an amino acid sequence selected from the group consisting of
(i) SEQ ID NO: 125, wherein Xaa at positions 51-53 and 96-98 are wild-type amino acids, wherein Xaa at position 54 is Thr;
(ii) SEQ ID NO: 125, wherein Xaa at positions 51 and 96-98 are wild-type amino acids, wherein Xaa at positions 52-54 are Ile, Gln, and Ile, respectively;
(iii) SEQ ID NO: 125, wherein Xaa at positions 51, 53, and 96-98 are wild-type amino acids, wherein Xaa at positions 52 and 54 are Ile and Thr, respectively;
(iv) SEQ ID NO: 125, wherein Xaa at positions 51, 52, and 96-98 are wild-type amino acids, wherein Xaa at positions 53 and 54 are Gln and Thr, respectively;
(v) SEQ ID NO: 125, wherein Xaa at positions 54 and 96-98 are wild-type amino acids, wherein Xaa at positions 51-53 are Ala, Ile, Gln, respectively;
(vi) SEQ ID NO: 125, wherein Xaa at positions 53 and 96-98 are wild-type amino acids, wherein Xaa at positions 51, 52, and 54 are Ala, Ile, and Thr, respectively;
(vii) SEQ ID NO: 125, wherein Xaa at positions 51 and 96-98 are wild-type amino acids, wherein Xaa at positions 52-54 are Ile, Gln, and Thr, respectively;
(viii) SEQ ID NO: 125, wherein Xaa at positions 96-98 are wild-type amino acids, wherein Xaa at positions 51-54 are Ala, Ile, Gln, and Thr respectively;
(ix) SEQ ID NO: 125, wherein Xaa at positions 51-54, 96, and 97 are wild-type amino acids, wherein Xaa at position 98 is Leu;
(x) SEQ NO: 126, wherein Xaa at positions 94-97 are wild-type amino acids, wherein Xaa at positions 51-53 are Thr, Pro, and Trp, respectively;
(xi) SEQ ID NO: 126, wherein Xaa at positions 52, 53, and 94-97 are wild-type amino acids, wherein Xaa at position 51 is Pro;
(xii) SEQ ID NO: 126, wherein Xaa at positions 52, 53, and 94-97 are wild-type amino acids, wherein Xaa at position 51 is Thr;
(xiii) SEQ ID NO: 126, wherein Xaa at positions 51, 53, and 94-97 are wild-type amino acids, wherein Xaa at position 52 is Phe;
(xiv) SEQ ID NO: 126, wherein Xaa at positions 51, 53, and 94-97 are wild-type amino acids, wherein Xaa at position 52 is Pro;
(xv) SEQ ID NO: 126, wherein Xaa at positions 51, 52, and 94-97 are wild-type amino acids, wherein Xaa at position 53 is Trp;
(xvi) SEQ ID NO: 126, wherein Xaa at positions 53 and 94-97 are wild-type amino acids, wherein Xaa at positions 51 and 52 are Pro and Phe, respectively;
(xvii) SEQ ID NO: 126, wherein Xaa at positions 52 and 94-97 are wild-type amino acids, wherein Xaa at positions 51 and 53 are Pro and Trp;
(xviii) SEQ ID NO: 126, wherein Xaa at positions 51 and 94-97 are wild-type amino acids, wherein Xaa at positions 52 and 53 are Phe and Trp;
(xix) SEQ ID NO: 126, wherein Xaa at positions 53 and 94-97 are wild-type amino acids, wherein each Xaa at positions 51 and 52 is Pro, respectively;
(xx) SEQ ID NO: 126, wherein Xaa at positions 53 and 94-97 are wild-type amino acids, wherein Xaa at positions 51 and 52 is Thr and Pro, respectively;
(xxi) SEQ ID NO: 126, wherein Xaa at positions 52 and 94-97 are wild-type amino acids, wherein Xaa at positions 51 and 53 are Thr and Trp, respectively;
(xxii) SEQ ID NO: 126, wherein Xaa at positions 51 and 94-97 are wildtype amino acids, wherein Xaa at positions 52 and 53 are Pro and Trp, respectively;
(xxiii) SEQ ID NO: 126, wherein Xaa at positions 53 and 94-97 are wild-type amino acids, wherein Xaa at positions 51 and 52 are Thr and Phe, respectively;
(xxiv) SEQ ID NO: 126, wherein Xaa at positions 94-97 are wild-type amino acids, wherein Xaa at positions 51-53 are Pro, Phe, and Trp, respectively;
(xxv) SEQ ID NO: 126, wherein Xaa at positions 51-53, 94, and 97 are wild-type amino acids, wherein Xaa at positions 95 and 96 are Leu and Tyr, respectively;
(xxvi) SEQ ID NO: 126, wherein Xaa at positions 51-53, 94, 96, and 97 are wild-type amino acids, wherein Xaa at position 95 is Ala;
(xxvii) SEQ ID NO: 126, wherein Xaa at positions 51-53, 94, 96, and 97 are wild-type amino acids, wherein Xaa at position 95 is Leu;
(xxviii) SEQ ID NO: 126, wherein Xaa at positions 51-53, 94, 96, and 97 are wild-type amino acids, wherein Xaa at position 95 is Glu;
(xxix) SEQ ID NO: 126, wherein Xaa at positions 51-53, 94, 95, and 97 are wild-type amino acids, wherein Xaa at position 96 is Ala;
(xxx) SEQ ID NO: 126, wherein Xaa at positions 51-53, 94, 95, and 97 are wild-type amino acids, wherein Xaa at position 96 is Leu;
(xxxi) SEQ ID NO: 126, wherein Xaa at positions 51-53, 94, 95, and 97 are wild-type amino acids, wherein Xaa at position 96 is Tyr;
(xxxii) SEQ ID NO: 126, wherein Xaa at positions 51-53, 94, 95, and 97 are wild-type amino acids, wherein Xaa at position 96 is Glu;
(xxxiii) SEQ ID NO: 126, wherein Xaa at positions 51-53, 94, 95, and 97 are wild-type amino acids, wherein Xaa at position 96 is Lys;
(xxxiv) SEQ ID NO: 126, wherein Xaa at positions 51-53 and 94 are wild-type amino acids, wherein Xaa at positions 95-97 are Leu, Leu, and Asp, respectively;
(xxxv) SEQ ID NO: 126, wherein Xaa at positions 51-53, 94, and 97 are wild-type amino acids, wherein each Xaa at positions 95 and 96 are Leu;
(xxxvi) SEQ ID NO: 126, wherein Xaa at positions 51-53, 94, and 97 are wild-type amino acids, wherein Xaa at positions 95 and 96 are Glu and Tyr, respectively;
(xxxvii) SEQ ID NO: 126, wherein Xaa at positions 51-53, 94, and 97 are wild-type amino acids, wherein Xaa at positions 95 and 96 are Trp and Val, respectively;
(xxxviii) SEQ ID NO: 126, wherein Xaa at positions 51-53, 96, and 97 are wild-type amino acids, wherein Xaa at positions 94 and 95 are Leu and Pro, respectively;
(xxxix) SEQ ID NO: 131, wherein Xaa at positions 52 to 54 are wild-type amino acids, wherein Xaa at position 51 is Val; and
(xxxx) SEQ ID NO: 131, wherein Xaa at positions 51, 53, and 54 are wild-type amino acids, wherein Xaa at position 52 is Met;
or a combination thereof;
wherein the wild-type amino acids of SEQ ID NO: 125 at positions 51-54 and 96-98 are Gly, Ala, Gly, Ile, Val, Gly, and Asn, respectively,
wherein the wild-type amino acids of SEQ ID NO: 126 at positions 51-53 and 94-97 are Gln, Ser, Ser, Pro, Thr, Ser, and Gly, respectively,
wherein the wild-type amino acids of SEQ ID NO: 131 at positions 51-54 are Thr, Ala, Gly, and Thr, respectively.

6. The isolated protein of claim 5, wherein, when the protein comprises an amino acid sequence selected from the group consisting of (i) to (ix), the protein further comprises SEQ ID NO: 122, when the protein comprises an amino acid sequence selected from the group consisting of (x) to (xxviii), the protein further comprises SEQ ID NO: 121, and when the protein comprises an amino acid sequence selected from the group consisting of (xxviii) and (xxix), the protein further comprises SEQ ID NO: 13, or a mature form thereof.

7. The isolated protein of claim 5, wherein, when the protein comprises the amino acid sequence of (x) or (xxv), the protein further comprises the amino acid sequence of SEQ ID NO: 125, wherein Xaa at positions 51, 53, 54, and 96-98 are wild-type amino acids and Xaa at position 52 is Ile, or SEQ ID NO: 125, wherein Xaa at positions 53, 54, and 96-98 are wild-type amino acids and Xaa at positions 51 and 52 are Ala and Ile, respectively.

8. The isolated protein of claim 5, wherein the protein comprises an amino acid sequence selected from the group consisting of
(i) SEQ ID NO: 5, wherein Xaa at positions 70-72 and 115-117 are wild-type amino acids, wherein Xaa at position 73 is Thr;
(ii) SEQ ID. NO: 5, wherein Xaa at positions 70 and 115-117 are wild-type amino acids, wherein Xaa at positions 71-73 are Ile, Gln, and Ile, respectively;
(iii) SEQ ID NO: 5, wherein Xaa at positions 70, 72, and 115-117 are wild-type amino acids, wherein Xaa at positions 71 and 73 are Ile and Thr, respectively;
(iv) SEQ ID NO: 5, wherein Xaa at positions 70, 71, and 115-117 are wild-type amino acids, wherein Xaa at positions 72 and 73 are Gln and Thr, respectively;
(v) SEQ ID NO: 5, wherein Xaa at positions 73 and 115-117 are wild-type amino acids, wherein Xaa at positions 70-72 are Ala, Ile, Gln, respectively;
(vi) SEQ ID NO: 5, wherein Xaa at positions 72 and 115-117 are wild-type amino acids, wherein Xaa at positions 70, 71, and 73 are Ala, Ile, and Thr, respectively;
(vii) SEQ ID NO: 5, wherein Xaa at positions 70 and 115-117 are wild-type amino acids, wherein Xaa at positions 71-73 are Ile, Gln, and Thr, respectively;
(viii) SEQ ID NO: 5, wherein Xaa at positions 115-117 are wild-type amino acids, wherein Xaa at positions 70-73 are Ala, Ile, Gln, and Thr respectively;
(ix) SEQ ID NO: 5, wherein Xaa at positions 70-73, 115, and 116 are wild-type amino acids, wherein Xaa at position 117 is Leu;
(x) SEQ ID NO: 6, wherein Xaa at positions 113-116 are wild-type amino acids, wherein Xaa at positions 70-72 are Thr, Pro, and Trp, respectively;
(xi) SEQ ID NO: 6, wherein Xaa at positions 71, 72, and 113-116 are wild-type amino acids, wherein Xaa at position 70 is Pro;
(xii) SEQ ID NO: 6, wherein Xaa at positions 71, 72, and 113-116 are wild-type amino acids, wherein Xaa at position 70 is Thr;
(xiii) SEQ ID NO: 6, wherein Xaa at positions 70, 72, and 113-116 are wild-type amino acids, wherein Xaa at position 71 is Phe;
(xiv) SEQ ID NO: 6, wherein Xaa at positions 70, 72, and 113-116 are wild-type amino acids, wherein Xaa at position 71 is Pro;
(xv) SEQ ID NO: 6, wherein Xaa at positions 70, 71, and 113-116 are wild-type amino acids, wherein Xaa at position 72 is Trp;
(xvi) SEQ ID NO: 6, wherein Xaa at positions 72 and 113-116 are wild-type amino acids, wherein Xaa at positions 70 and 71 are Pro and Phe, respectively;
(xvii) SEQ ID NO: 6, wherein Xaa at positions 71 and 113-116 are wild-type amino acids, wherein Xaa at positions 70 and 72 are Pro and Trp;
(xviii) SEQ ID NO: 6, wherein Xaa at positions 70 and 113-116 are wild-type amino acids, wherein Xaa at positions 71 and 72 are Phe and Trp;
(xix) SEQ ID NO: 6, wherein Xaa at positions 72 and 113-116 are wild-type amino acids, wherein each Xaa at positions 70 and 71 is Pro, respectively;
(xx) SEQ ID NO: 6, wherein Xaa at positions 72 and 113-116 are wild-type amino acids, wherein Xaa at positions 70 and 71 is Thr and Pro, respectively;
(xxi) SEQ ID NO: 6, wherein Xaa at positions 71 and 113-116 are wild-type amino acids, wherein Xaa at positions 70 and 72 are Thr and Trp, respectively;
(xxii) SEQ ID NO: 6, wherein Xaa at positions 70 and 113-116 are wildtype amino acids, wherein Xaa at positions 71 and 72 are Pro and Trp, respectively;
(xxiii) SEQ ID NO: 6, wherein Xaa at positions 72 and 113-116 are wild-type amino acids, wherein Xaa at positions 70 and 71 are Thr and Phe, respectively;
(xxiv) SEQ ID NO: 6, wherein Xaa at positions 113-116 are wild-type amino acids, wherein Xaa at positions 70 to 72 are Pro, Phe, and Trp, respectively;
(xxv) SEQ ID NO: 6, wherein Xaa at positions 70-72, 113, and 116 are wild-type amino acids, wherein Xaa at positions 114 and 115 are Leu and Tyr, respectively;
(xxvi) SEQ ID NO: 6, wherein Xaa at positions 70-72, 113, 115, and 116 are wild-type amino acids, wherein Xaa at position 114 is Ala;
(xxvii) SEQ ID NO: 6, wherein Xaa at positions 70-72, 113, 115, and 116 are wild-type amino acids, wherein Xaa at position 114 is Leu;
(xxviii) SEQ ID. NO: 6, wherein Xaa at positions 70-72, 113, 115, and 116 are wild-type amino acids, wherein Xaa at position 114 is Glu;
(xxix) SEQ ID NO: 6, wherein Xaa at positions 70-72, 113, 114, and 116 are wild-type amino acids, wherein Xaa at position 115 is Ala;
(xxx) SEQ ID NO: 6, wherein Xaa at positions 70-72, 113, 114, and 116 are wild-type amino acids, wherein Xaa at position 115 is Leu;
(xxxi) SEQ ID NO: 6 wherein Xaa at positions 70-72, 113, 114, and 116 are wild-type amino acids, wherein Xaa at position 115 is Tyr;
(xxxii) SEQ ID NO: 6, wherein Xaa at positions 70-72, 113, 114, and 116 are wild-type amino acids, wherein Xaa at position 115 is Glu;
(xxxiii) SEQ ID NO: 6, wherein Xaa at positions 70-72, 113, 114, and 116 are wild-type amino acids, wherein Xaa at position 115 is Lys;
(xxxiv) SEQ ID NO: 6, wherein Xaa at positions 70-72 and 113 are wild-type amino acids, wherein Xaa at positions 114-116 are Leu, Leu, and Asp, respectively;
(xxxv) SEQ ID NO: 6, wherein Xaa at positions 70-72, 113, and 116 are wild-type amino acids, wherein each Xaa at positions 114 and 115 are Leu;
(xxxvi) SEQ ID NO: 6, wherein Xaa at positions 70-72, 113, and 116 are wild-type amino acids, wherein Xaa at positions 114 and 115 are Glu and Tyr, respectively;
(xxxvii) SEQ ID NO: 6, wherein Xaa at positions 70-72, 115, and 116 are wild-type amino acids, wherein Xaa at positions 113 and 114 are Leu and Pro, respectively;
(xxxviii) SEQ ID NO: 11, wherein Xaa at positions 71 to 73 are wild-type amino acids, wherein Xaa at position 70 is Val; and
(xxxix) SEQ ID NO: 11, wherein Xaa at positions 70, 72, and 73 are wild-type amino acids, wherein Xaa at position 71 is Met;
or a combination thereof;
wherein the wild-type amino acids of SEQ ID NO: 5 at positions 70-73 and 115-117 are Gly, Ala, Gly, Ile, Val, Gly, and Asn, respectively,
wherein the wild-type amino acids of SEQ ID NO: 6 at positions 70-72 and 113-116 are Gln, Ser, Ser, Pro, Thr, Ser, and Gly, respectively,
wherein the wild-type amino acids of SEQ ID NO: 11 at positions 70-73 are Thr, Ala, Gly, and Thr, respectively.

9. An isolated nucleic acid comprising a nucleotide sequence encoding the protein of claim 1, wherein the nucleic acid optionally is incorporated into a recombinant expression vector.

10. An isolated host cell comprising the nucleic acid of claim 9.

11. The isolated host cell of claim 10, wherein the cell is a peripheral blood lymphocyte (PBL).

12. The isolated host cell of claim 11, wherein the PBL is a CD8+ T cell or a CD4+ T cell.

13. A method of treating or preventing a disease in a mammal, comprising administering to the mammal a pharmaceutical composition comprising a population of cells comprising the host cells of claim 10 in an amount effective to treat or prevent the disease in the mammal.

14. The method of claim 13, wherein the disease is a cancer or an infectious disease.

15. The method of claim 14, wherein the cancer is melanoma.

16. The method of claim 13, wherein the mammal is a human.

17. The method of claim 13, wherein the host cells are cells which are autologous to the mammal.

18. A method of detecting a diseased cell in a mammal, wherein the diseased cell expresses an antigen characteristic of a disease, comprising (i) contacting a sample comprising cells of the mammal with the protein of claim 1, thereby forming a complex between the antigen and the protein, and (ii) detecting the complex, wherein detecting of the complex is indicative of a diseased cell in the mammal.

19. The method of claim 18, wherein the diseased cell is a cancer cell or an infected cell.

20. The method of claim 19, wherein the diseased cell is a melanoma cell.
